SEQUENCE LISTING (744 sequences)

SEQ ID NO: 1 - DNA, 58 bp, Artificial Sequence
  Description of Artificial Sequence Synthetic polynucleotide
aatgatacggcgaccaccgagatctacactctttccctacacgacgctcttccgatct

SEQ ID NO: 2 - DNA, 12 bp, Artificial Sequence
  Description of Artificial Sequence Synthetic polynucleotide
tgttcttcgtaa

SEQ ID NO: 3 - DNA, 94 bp, Artificial Sequence
  Description of Artificial Sequence Synthetic polynucleotide
  modified_base (75)..(94): a, c, t, g, unknown or other
  See specification as filed for detailed description of substitutions and preferred embodiments
aatgatacggcgaccaccgagatctacactgttcttcgtaaacactctttccctacacgacgctcttccgatctnnnnnnnnnnnnnnnnnnnn

SEQ ID NO: 4 - DNA, 29 bp, Artificial Sequence
  Description of Artificial Sequence Synthetic polynucleotide
aatgatacggcgaccaccgagatctacac

SEQ ID NO: 5 - DNA, 53 bp, Artificial Sequence
  Description of Artificial Sequence Synthetic polynucleotide
  modified_base (34)..(53): a, c, t, g, unknown or other
  See specification as filed for detailed description of substitutions and preferred embodiments
acactctttccctacacgacgctcttccgatctnnnnnnnnnnnnnnnnnnnn

SEQ ID NO: 6 - DNA, 12 bp, Artificial Sequence
  Description of Artificial Sequence Synthetic polynucleotide
attgattattcg

SEQ ID NO: 7 - DNA, 12 bp, Artificial Sequence
  Description of Artificial Sequence Synthetic polynucleotide
gctctgccggct

SEQ ID NO: 8 - DNA, 12 bp, Artificial Sequence
  Description of Artificial Sequence Synthetic polynucleotide
gattcgttagcg

SEQ ID NO: 9 - DNA, 12 bp, Artificial Sequence
  Description of Artificial Sequence Synthetic polynucleotide
tacaagcgaggt

SEQ ID NO: 10 - DNA, 12 bp, Artificial Sequence
  Description of Artificial Sequence Synthetic polynucleotide
ttccaggtcgcg

SEQ ID NO: 11 - DNA, 12 bp, Artificial Sequence
  Description of Artificial Sequence Synthetic polynucleotide
agtaactagtcg

SEQ ID NO: 12 - DNA, 12 bp, Artificial Sequence
  Description of Artificial Sequence Synthetic polynucleotide
aatctcgaagtc

SEQ ID NO: 13 - DNA, 12 bp, Artificial Sequence
  Description of Artificial Sequence Synthetic polynucleotide
ccgggaatgtta

SEQ ID NO: 14 - DNA, 12 bp, Artificial Sequence
  Description of Artificial Sequence Synthetic polynucleotide
ccgttggatttg

SEQ ID NO: 15 - DNA, 12 bp, Artificial Sequence
  Description of Artificial Sequence Synthetic polynucleotide
ccacgaggtgac

SEQ ID NO: 16 - DNA, 12 bp, Artificial Sequence
  Description of Artificial Sequence Synthetic polynucleotide
cgcgatccatct

SEQ ID NO: 17 - DNA, 12 bp, Artificial Sequence
  Description of Artificial Sequence Synthetic polynucleotide
ctccgtgggagt

SEQ ID NO: 18 - DNA, 12 bp, Artificial Sequence
  Description of Artificial Sequence Synthetic polynucleotide
gcaagacccaat

SEQ ID NO: 19 - DNA, 12 bp, Artificial Sequence
  Description of Artificial Sequence Synthetic polynucleotide
ttgtgaccttac

SEQ ID NO: 20 - DNA, 12 bp, Artificial Sequence
  Description of Artificial Sequence Synthetic polynucleotide
ataccgatagtg

SEQ ID NO: 21 - DNA, 12 bp, Artificial Sequence
  Description of Artificial Sequence Synthetic polynucleotide
tgatcagcgatc

SEQ ID NO: 22 - DNA, 12 bp, Artificial Sequence
  Description of Artificial Sequence Synthetic polynucleotide
cccaggcttctc

SEQ ID NO: 23 - DNA, 12 bp, Artificial Sequence
  Description of Artificial Sequence Synthetic polynucleotide
cccagcgcaata

SEQ ID NO: 24 - DNA, 12 bp, Artificial Sequence
  Description of Artificial Sequence Synthetic polynucleotide
ctactagaaata

SEQ ID NO: 25 - DNA, 12 bp, Artificial Sequence
  Description of Artificial Sequence Synthetic polynucleotide
cggaagggctac

SEQ ID NO: 26 - DNA, 12 bp, Artificial Sequence
  Description of Artificial Sequence Synthetic polynucleotide
tggacggctgtg

SEQ ID NO: 27 - DNA, 12 bp, Artificial Sequence
  Description of Artificial Sequence Synthetic polynucleotide
gtgcaaggcaca

SEQ ID NO: 28 - DNA, 12 bp, Artificial Sequence
  Description of Artificial Sequence Synthetic polynucleotide
cgagtcttgacg

SEQ ID NO: 29 - DNA, 12 bp, Artificial Sequence
  Description of Artificial Sequence Synthetic polynucleotide
gatcgcgttgag

SEQ ID NO: 30 - DNA, 12 bp, Artificial Sequence
  Description of Artificial Sequence Synthetic polynucleotide
tatgatgcagga

SEQ ID NO: 31 - DNA, 12 bp, Artificial Sequence
  Description of Artificial Sequence Synthetic polynucleotide
acagataaccat

SEQ ID NO: 32 - DNA, 12 bp, Artificial Sequence
  Description of Artificial Sequence Synthetic polynucleotide
gtatgtctgaaa

SEQ ID NO: 33 - DNA, 12 bp, Artificial Sequence
  Description of Artificial Sequence Synthetic polynucleotide
ctcagctttaaa

SEQ ID NO: 34 - DNA, 12 bp, Artificial Sequence
  Description of Artificial Sequence Synthetic polynucleotide
gaattctaggtt

SEQ ID NO: 35 - DNA, 12 bp, Artificial Sequence
  Description of Artificial Sequence Synthetic polynucleotide
tgcgcgaccact

SEQ ID NO: 36 - DNA, 12 bp, Artificial Sequence
  Description of Artificial Sequence Synthetic polynucleotide
acaccttccgca

SEQ ID NO: 37 - DNA, 12 bp, Artificial Sequence
  Description of Artificial Sequence Synthetic polynucleotide
tatactcaggat

SEQ ID NO: 38 - DNA, 12 bp, Artificial Sequence
  Description of Artificial Sequence Synthetic polynucleotide
tcaatccaaacg

SEQ ID NO: 39 - DNA, 12 bp, Artificial Sequence
  Description of Artificial Sequence Synthetic polynucleotide
ataggtgaatgt

SEQ ID NO: 40 - DNA, 12 bp, Artificial Sequence
  Description of Artificial Sequence Synthetic polynucleotide
tccaattcgacg

SEQ ID NO: 41 - DNA, 12 bp, Artificial Sequence
  Description of Artificial Sequence Synthetic polynucleotide
catcgctgatca

SEQ ID NO: 42 - DNA, 12 bp, Artificial Sequence
  Description of Artificial Sequence Synthetic polynucleotide
cgggtaaattgt

SEQ ID NO: 43 - DNA, 12 bp, Artificial Sequence
  Description of Artificial Sequence Synthetic polynucleotide
ctactaacccag

SEQ ID NO: 44 - DNA, 12 bp, Artificial Sequence
  Description of Artificial Sequence Synthetic polynucleotide
gacatccgagga

SEQ ID NO: 45 - DNA, 12 bp, Artificial Sequence
  Description of Artificial Sequence Synthetic polynucleotide
ccacctccttgt

SEQ ID NO: 46 - DNA, 12 bp, Artificial Sequence
  Description of Artificial Sequence Synthetic polynucleotide
acttggaagttc

SEQ ID NO: 47 - DNA, 12 bp, Artificial Sequence
  Description of Artificial Sequence Synthetic polynucleotide
tccaagcagctg

SEQ ID NO: 48 - DNA, 12 bp, Artificial Sequence
  Description of Artificial Sequence Synthetic polynucleotide
attcaccttcgc

SEQ ID NO: 49 - DNA, 12 bp, Artificial Sequence
  Description of Artificial Sequence Synthetic polynucleotide
ctcgtttccaac

SEQ ID NO: 50 - DNA, 12 bp, Artificial Sequence
  Description of Artificial Sequence Synthetic polynucleotide
tccgagggtaca

SEQ ID NO: 51 - DNA, 12 bp, Artificial Sequence
  Description of Artificial Sequence Synthetic polynucleotide
gtaatatagcct

SEQ ID NO: 52 - DNA, 12 bp, Artificial Sequence
  Description of Artificial Sequence Synthetic polynucleotide
gtaataggttat

SEQ ID NO: 53 - DNA, 12 bp, Artificial Sequence
  Description of Artificial Sequence Synthetic polynucleotide
cgcattgtattt

SEQ ID NO: 54 - DNA, 12 bp, Artificial Sequence
  Description of Artificial Sequence Synthetic polynucleotide
tcatggattacg

SEQ ID NO: 55 - DNA, 12 bp, Artificial Sequence
  Description of Artificial Sequence Synthetic polynucleotide
actcacgaagct

SEQ ID NO: 56 - DNA, 12 bp, Artificial Sequence
  Description of Artificial Sequence Synthetic polynucleotide
caattcgccaaa

SEQ ID NO: 57 - DNA, 12 bp, Artificial Sequence
  Description of Artificial Sequence Synthetic polynucleotide
aacattctatac

SEQ ID NO: 58 - DNA, 12 bp, Artificial Sequence
  Description of Artificial Sequence Synthetic polynucleotide
caacacttgaaa

SEQ ID NO: 59 - DNA, 12 bp, Artificial Sequence
  Description of Artificial Sequence Synthetic polynucleotide
agagagtgtctc

SEQ ID NO: 60 - DNA, 12 bp, Artificial Sequence
  Description of Artificial Sequence Synthetic polynucleotide
ttcatttggctc

SEQ ID NO: 61 - DNA, 12 bp, Artificial Sequence
  Description of Artificial Sequence Synthetic polynucleotide
gcgttggtaccg

SEQ ID NO: 62 - DNA, 12 bp, Artificial Sequence
  Description of Artificial Sequence Synthetic polynucleotide
cactgacggtga

SEQ ID NO: 63 - DNA, 12 bp, Artificial Sequence
  Description of Artificial Sequence Synthetic polynucleotide
cggtcactgaat

SEQ ID NO: 64 - DNA, 12 bp, Artificial Sequence
  Description of Artificial Sequence Synthetic polynucleotide
cctctgagatag

SEQ ID NO: 65 - DNA, 12 bp, Artificial Sequence
  Description of Artificial Sequence Synthetic polynucleotide
atcgcatcattg

SEQ ID NO: 66 - DNA, 12 bp, Artificial Sequence
  Description of Artificial Sequence Synthetic polynucleotide
cagtttcagcgc

SEQ ID NO: 67 - DNA, 12 bp, Artificial Sequence
  Description of Artificial Sequence Synthetic polynucleotide
atcggttatatt

SEQ ID NO: 68 - DNA, 12 bp, Artificial Sequence
  Description of Artificial Sequence Synthetic polynucleotide
tcgggagacgct

SEQ ID NO: 69 - DNA, 12 bp, Artificial Sequence
  Description of Artificial Sequence Synthetic polynucleotide
gcttctttattt

SEQ ID NO: 70 - DNA, 12 bp, Artificial Sequence
  Description of Artificial Sequence Synthetic polynucleotide
cagcccaagagc

SEQ ID NO: 71 - DNA, 12 bp, Artificial Sequence
  Description of Artificial Sequence Synthetic polynucleotide
cgtatttccgat

SEQ ID NO: 72 - DNA, 12 bp, Artificial Sequence
  Description of Artificial Sequence Synthetic polynucleotide
aggaatgtctgt

SEQ ID NO: 73 - DNA, 12 bp, Artificial Sequence
  Description of Artificial Sequence Synthetic polynucleotide
ttacgcttgact

SEQ ID NO: 74 - DNA, 12 bp, Artificial Sequence
  Description of Artificial Sequence Synthetic polynucleotide
cgagaaaggtgt

SEQ ID NO: 75 - DNA, 12 bp, Artificial Sequence
  Description of Artificial Sequence Synthetic polynucleotide
taattcaagctg

SEQ ID NO: 76 - DNA, 12 bp, Artificial Sequence
  Description of Artificial Sequence Synthetic polynucleotide
tggacggtcaac

SEQ ID NO: 77 - DNA, 12 bp, Artificial Sequence
  Description of Artificial Sequence Synthetic polynucleotide
gtaacggtgggt

SEQ ID NO: 78 - DNA, 12 bp, Artificial Sequence
  Description of Artificial Sequence Synthetic polynucleotide
atcacggctggc

SEQ ID NO: 79 - DNA, 12 bp, Artificial Sequence
  Description of Artificial Sequence Synthetic polynucleotide
taacattcgcgt

SEQ ID NO: 80 - DNA, 12 bp, Artificial Sequence
  Description of Artificial Sequence Synthetic polynucleotide
atctggttatgc

SEQ ID NO: 81 - DNA, 12 bp, Artificial Sequence
  Description of Artificial Sequence Synthetic polynucleotide
gcgctacccagc

SEQ ID NO: 82 - DNA, 12 bp, Artificial Sequence
  Description of Artificial Sequence Synthetic polynucleotide
agcgtgggagtt

SEQ ID NO: 83 - DNA, 12 bp, Artificial Sequence
  Description of Artificial Sequence Synthetic polynucleotide
tagaaggagcta

SEQ ID NO: 84 - DNA, 12 bp, Artificial Sequence
  Description of Artificial Sequence Synthetic polynucleotide
ttcgtacagctc

SEQ ID NO: 85 - DNA, 12 bp, Artificial Sequence
  Description of Artificial Sequence Synthetic polynucleotide
tttaggacgaga

SEQ ID NO: 86 - DNA, 12 bp, Artificial Sequence
  Description of Artificial Sequence Synthetic polynucleotide
ttggcagaacga

SEQ ID NO: 87 - DNA, 12 bp, Artificial Sequence
  Description of Artificial Sequence Synthetic polynucleotide
acatgactcatt

SEQ ID NO: 88 - DNA, 12 bp, Artificial Sequence
  Description of Artificial Sequence Synthetic polynucleotide
ccagttcgatga

SEQ ID NO: 89 - DNA, 12 bp, Artificial Sequence
  Description of Artificial Sequence Synthetic polynucleotide
tccgtacgtcga

SEQ ID NO: 90 - DNA, 12 bp, Artificial Sequence
  Description of Artificial Sequence Synthetic polynucleotide
tcggctgacatc

SEQ ID NO: 91 - DNA, 12 bp, Artificial Sequence
  Description of Artificial Sequence Synthetic polynucleotide
cgttgggacttt

SEQ ID NO: 92 - DNA, 12 bp, Artificial Sequence
  Description of Artificial Sequence Synthetic polynucleotide
tgcatctggata

SEQ ID NO: 93 - DNA, 12 bp, Artificial Sequence
  Description of Artificial Sequence Synthetic polynucleotide
tggccaaacctt

SEQ ID NO: 94 - DNA, 12 bp, Artificial Sequence
  Description of Artificial Sequence Synthetic polynucleotide
tctgctgccttg

SEQ ID NO: 95 - DNA, 12 bp, Artificial Sequence
  Description of Artificial Sequence Synthetic polynucleotide
ctcaggataact

SEQ ID NO: 96 - DNA, 12 bp, Artificial Sequence
  Description of Artificial Sequence Synthetic polynucleotide
tggcaaggaaag

SEQ ID NO: 97 - DNA, 12 bp, Artificial Sequence
  Description of Artificial Sequence Synthetic polynucleotide
acccgaagttta

SEQ ID NO: 98 - DNA, 12 bp, Artificial Sequence
  Description of Artificial Sequence Synthetic polynucleotide
agtaaactgcta

SEQ ID NO: 99 - DNA, 12 bp, Artificial Sequence
  Description of Artificial Sequence Synthetic polynucleotide
ttgcccataggt

SEQ ID NO: 100 - DNA, 12 bp, Artificial Sequence
  Description of Artificial Sequence Synthetic polynucleotide
agtggtagttag

SEQ ID NO: 101 - DNA, 12 bp, Artificial Sequence
  Description of Artificial Sequence Synthetic polynucleotide
caataaacgccg

SEQ ID NO: 102 - DNA, 12 bp, Artificial Sequence
  Description of Artificial Sequence Synthetic polynucleotide
ttgaccacgacg

SEQ ID NO: 103 - DNA, 12 bp, Artificial Sequence
  Description of Artificial Sequence Synthetic polynucleotide
gcactcctaaat

SEQ ID NO: 104 - DNA, 12 bp, Artificial Sequence
  Description of Artificial Sequence Synthetic polynucleotide
ccagacatatga

SEQ ID NO: 105 - DNA, 12 bp, Artificial Sequence
  Description of Artificial Sequence Synthetic polynucleotide
cattacccgtca

SEQ ID NO: 106 - DNA, 12 bp, Artificial Sequence
  Description of Artificial Sequence Synthetic polynucleotide
acggttgagaat

SEQ ID NO: 107 - DNA, 12 bp, Artificial Sequence
  Description of Artificial Sequence Synthetic polynucleotide
ctacctcagaat

SEQ ID NO: 108 - DNA, 12 bp, Artificial Sequence
  Description of Artificial Sequence Synthetic polynucleotide
tttgtacaccgt

SEQ ID NO: 109 - DNA, 12 bp, Artificial Sequence
  Description of Artificial Sequence Synthetic polynucleotide
ccgaacaggtaa

SEQ ID NO: 110 - DNA, 12 bp, Artificial Sequence
  Description of Artificial Sequence Synthetic polynucleotide
tgtcggattaaa

SEQ ID NO: 111 - DNA, 12 bp, Artificial Sequence
  Description of Artificial Sequence Synthetic polynucleotide
acggacctccta

SEQ ID NO: 112 - DNA, 12 bp, Artificial Sequence
  Description of Artificial Sequence Synthetic polynucleotide
tagataagtcag

SEQ ID NO: 113 - DNA, 12 bp, Artificial Sequence
  Description of Artificial Sequence Synthetic polynucleotide
aatttcggctta

SEQ ID NO: 114 - DNA, 12 bp, Artificial Sequence
  Description of Artificial Sequence Synthetic polynucleotide
aattgtttgaag

SEQ ID NO: 115 - DNA, 12 bp, Artificial Sequence
  Description of Artificial Sequence Synthetic polynucleotide
atggagctgaag

SEQ ID NO: 116 - DNA, 12 bp, Artificial Sequence
  Description of Artificial Sequence Synthetic polynucleotide
agctgttaccag

SEQ ID NO: 117 - DNA, 12 bp, Artificial Sequence
  Description of Artificial Sequence Synthetic polynucleotide
agcacccgattc

SEQ ID NO: 118 - DNA, 12 bp, Artificial Sequence
  Description of Artificial Sequence Synthetic polynucleotide
cctaacctcccg

SEQ ID NO: 119 - DNA, 12 bp, Artificial Sequence
  Description of Artificial Sequence Synthetic polynucleotide
agggctgtgctg

SEQ ID NO: 120 - DNA, 12 bp, Artificial Sequence
  Description of Artificial Sequence Synthetic polynucleotide
acatgctttgcg

SEQ ID NO: 121 - DNA, 12 bp, Artificial Sequence
  Description of Artificial Sequence Synthetic polynucleotide
tactgttgatat

SEQ ID NO: 122 - DNA, 12 bp, Artificial Sequence
  Description of Artificial Sequence Synthetic polynucleotide
cgtgcgcaccta

SEQ ID NO: 123 - DNA, 12 bp, Artificial Sequence
  Description of Artificial Sequence Synthetic polynucleotide
gagatctttagc

SEQ ID NO: 124 - DNA, 12 bp, Artificial Sequence
  Description of Artificial Sequence Synthetic polynucleotide
aatgcagcgcaa

SEQ ID NO: 125 - DNA, 12 bp, Artificial Sequence
  Description of Artificial Sequence Synthetic polynucleotide
tacaatggtcat

SEQ ID NO: 126 - DNA, 12 bp, Artificial Sequence
  Description of Artificial Sequence Synthetic polynucleotide
taagagactaca

SEQ ID NO: 127 - DNA, 12 bp, Artificial Sequence
  Description of Artificial Sequence Synthetic polynucleotide
cttaagatgatc

SEQ ID NO: 128 - DNA, 12 bp, Artificial Sequence
  Description of Artificial Sequence Synthetic polynucleotide
aatttgcccatt

SEQ ID NO: 129 - DNA, 12 bp, Artificial Sequence
  Description of Artificial Sequence Synthetic polynucleotide
ttactctgatca

SEQ ID NO: 130 - DNA, 12 bp, Artificial Sequence
  Description of Artificial Sequence Synthetic polynucleotide
agttgtcttgtt

SEQ ID NO: 131 - DNA, 12 bp, Artificial Sequence
  Description of Artificial Sequence Synthetic polynucleotide
gagtggaatgac

SEQ ID NO: 132 - DNA, 12 bp, Artificial Sequence
  Description of Artificial Sequence Synthetic polynucleotide
gtccgcttgcaa

SEQ ID NO: 133 - DNA, 12 bp, Artificial Sequence
  Description of Artificial Sequence Synthetic polynucleotide
gttacgtggtct

SEQ ID NO: 134 - DNA, 12 bp, Artificial Sequence
  Description of Artificial Sequence Synthetic polynucleotide
gatctgtggctt

SEQ ID NO: 135 - DNA, 12 bp, Artificial Sequence
  Description of Artificial Sequence Synthetic polynucleotide
ctaaagtctgta

SEQ ID NO: 136 - DNA, 12 bp, Artificial Sequence
  Description of Artificial Sequence Synthetic polynucleotide
tcgtaccggtct

SEQ ID NO: 137 - DNA, 12 bp, Artificial Sequence
  Description of Artificial Sequence Synthetic polynucleotide
gcccactcagac

SEQ ID NO: 138 - DNA, 12 bp, Artificial Sequence
  Description of Artificial Sequence Synthetic polynucleotide
aggatagtcatc

SEQ ID NO: 139 - DNA, 12 bp, Artificial Sequence
  Description of Artificial Sequence Synthetic polynucleotide
attgctcgggca

SEQ ID NO: 140 - DNA, 12 bp, Artificial Sequence
  Description of Artificial Sequence Synthetic polynucleotide
cacagacgttac

SEQ ID NO: 141 - DNA, 12 bp, Artificial Sequence
  Description of Artificial Sequence Synthetic polynucleotide
ccagggaataag

SEQ ID NO: 142 - DNA, 12 bp, Artificial Sequence
  Description of Artificial Sequence Synthetic polynucleotide
ttcccgaactac

SEQ ID NO: 143 - DNA, 12 bp, Artificial Sequence
  Description of Artificial Sequence Synthetic polynucleotide
aacgagccattt

SEQ ID NO: 144 - DNA, 12 bp, Artificial Sequence
  Description of Artificial Sequence Synthetic polynucleotide
gcgcgtcctttc

SEQ ID NO: 145 - DNA, 12 bp, Artificial Sequence
  Description of Artificial Sequence Synthetic polynucleotide
tctatcgtgttc

SEQ ID NO: 146 - DNA, 12 bp, Artificial Sequence
  Description of Artificial Sequence Synthetic polynucleotide
aaacgctaagga

SEQ ID NO: 147 - DNA, 12 bp, Artificial Sequence
  Description of Artificial Sequence Synthetic polynucleotide
tgacacagatct

SEQ ID NO: 148 - DNA, 12 bp, Artificial Sequence
  Description of Artificial Sequence Synthetic polynucleotide
ccctacctctca

SEQ ID NO: 149 - DNA, 12 bp, Artificial Sequence
  Description of Artificial Sequence Synthetic polynucleotide
tgcggatccgca

SEQ ID NO: 150 - DNA, 12 bp, Artificial Sequence
  Description of Artificial Sequence Synthetic polynucleotide
gtcaacaacgac

SEQ ID NO: 151 - DNA, 12 bp, Artificial Sequence
  Description of Artificial Sequence Synthetic polynucleotide
aggaccacccag

SEQ ID NO: 152 - DNA, 12 bp, Artificial Sequence
  Description of Artificial Sequence Synthetic polynucleotide
ccaagtacattc

SEQ ID NO: 153 - DNA, 12 bp, Artificial Sequence
  Description of Artificial Sequence Synthetic polynucleotide
gtgctgtttcct

SEQ ID NO: 154 - DNA, 12 bp, Artificial Sequence
  Description of Artificial Sequence Synthetic polynucleotide
caaatgctagaa

SEQ ID NO: 155 - DNA, 12 bp, Artificial Sequence
  Description of Artificial Sequence Synthetic polynucleotide
atcatacaagga

SEQ ID NO: 156 - DNA, 12 bp, Artificial Sequence
  Description of Artificial Sequence Synthetic polynucleotide
catagtaagtag

SEQ ID NO: 157 - DNA, 12 bp, Artificial Sequence
  Description of Artificial Sequence Synthetic polynucleotide
cttaataaagag

SEQ ID NO: 158 - DNA, 12 bp, Artificial Sequence
  Description of Artificial Sequence Synthetic polynucleotide
gtacgctcaccg

SEQ ID NO: 159 - DNA, 12 bp, Artificial Sequence
  Description of Artificial Sequence Synthetic polynucleotide
cccgataaatgt

SEQ ID NO: 160 - DNA, 12 bp, Artificial Sequence
  Description of Artificial Sequence Synthetic polynucleotide
atcgcttaatac

SEQ ID NO: 161 - DNA, 12 bp, Artificial Sequence
  Description of Artificial Sequence Synthetic polynucleotide
ttgacgcattac

SEQ ID NO: 162 - DNA, 12 bp, Artificial Sequence
  Description of Artificial Sequence Synthetic polynucleotide
ttagtaggcgag

SEQ ID NO: 163 - DNA, 12 bp, Artificial Sequence
  Description of Artificial Sequence Synthetic polynucleotide
gttcactcacaa

SEQ ID NO: 164 - DNA, 12 bp, Artificial Sequence
  Description of Artificial Sequence Synthetic polynucleotide
catcgggccatg

SEQ ID NO: 165 - DNA, 12 bp, Artificial Sequence
  Description of Artificial Sequence Synthetic polynucleotide
aattacccagct

SEQ ID NO: 166 - DNA, 12 bp, Artificial Sequence
  Description of Artificial Sequence Synthetic polynucleotide
cgcatcaattcg

SEQ ID NO: 167 - DNA, 12 bp, Artificial Sequence
  Description of Artificial Sequence Synthetic polynucleotide
tcaacccgtcat

SEQ ID NO: 168 - DNA, 12 bp, Artificial Sequence
  Description of Artificial Sequence Synthetic polynucleotide
actagttcgagt

SEQ ID NO: 169 - DNA, 12 bp, Artificial Sequence
  Description of Artificial Sequence Synthetic polynucleotide
tacaggacgctg

SEQ ID NO: 170 - DNA, 12 bp, Artificial Sequence
  Description of Artificial Sequence Synthetic polynucleotide
aatgggctcgat

SEQ ID NO: 171 - DNA, 12 bp, Artificial Sequence
  Description of Artificial Sequence Synthetic polynucleotide
caccttgacaac

SEQ ID NO: 172 - DNA, 12 bp, Artificial Sequence
  Description of Artificial Sequence Synthetic polynucleotide
actcaccactgc

SEQ ID NO: 173 - DNA, 12 bp, Artificial Sequence
  Description of Artificial Sequence Synthetic polynucleotide
tagttcacacag

SEQ ID NO: 174 - DNA, 12 bp, Artificial Sequence
  Description of Artificial Sequence Synthetic polynucleotide
cttgcgttgagt

SEQ ID NO: 175 - DNA, 12 bp, Artificial Sequence
  Description of Artificial Sequence Synthetic polynucleotide
gctgatcccatc

SEQ ID NO: 176 - DNA, 12 bp, Artificial Sequence
  Description of Artificial Sequence Synthetic polynucleotide
tgagttctagat

SEQ ID NO: 177 - DNA, 12 bp, Artificial Sequence
  Description of Artificial Sequence Synthetic polynucleotide
caatctccatct

SEQ ID NO: 178 - DNA, 12 bp, Artificial Sequence
  Description of Artificial Sequence Synthetic polynucleotide
aggcaaagcaga

SEQ ID NO: 179 - DNA, 12 bp, Artificial Sequence
  Description of Artificial Sequence Synthetic polynucleotide
catgttaacgca

SEQ ID NO: 180 - DNA, 12 bp, Artificial Sequence
  Description of Artificial Sequence Synthetic polynucleotide
tgccgattacca

SEQ ID NO: 181 - DNA, 12 bp, Artificial Sequence
  Description of Artificial Sequence Synthetic polynucleotide
gtgacctatcca

SEQ ID NO: 182 - DNA, 12 bp, Artificial Sequence
  Description of Artificial Sequence Synthetic polynucleotide
agtggtaagtgc

SEQ ID NO: 183 - DNA, 12 bp, Artificial Sequence
  Description of Artificial Sequence Synthetic polynucleotide
agggaacatcgt

SEQ ID NO: 184 - DNA, 12 bp, Artificial Sequence
  Description of Artificial Sequence Synthetic polynucleotide
cgccatgacttc

SEQ ID NO: 185 - DNA, 12 bp, Artificial Sequence
  Description of Artificial Sequence Synthetic polynucleotide
atctcgccaaac

SEQ ID NO: 186 - DNA, 12 bp, Artificial Sequence
  Description of Artificial Sequence Synthetic polynucleotide
cgcctggccgta

SEQ ID NO: 187 - DNA, 12 bp, Artificial Sequence
  Description of Artificial Sequence Synthetic polynucleotide
agggtgctgtcg

SEQ ID NO: 188 - DNA, 12 bp, Artificial Sequence
  Description of Artificial Sequence Synthetic polynucleotide
agcctccgccct

SEQ ID NO: 189 - DNA, 12 bp, Artificial Sequence
  Description of Artificial Sequence Synthetic polynucleotide
gtttaatccaag

SEQ ID NO: 190 - DNA, 12 bp, Artificial Sequence
  Description of Artificial Sequence Synthetic polynucleotide
cagctatgacct

SEQ ID NO: 191 - DNA, 12 bp, Artificial Sequence
  Description of Artificial Sequence Synthetic polynucleotide
acgactggcact

SEQ ID NO: 192 - DNA, 12 bp, Artificial Sequence
  Description of Artificial Sequence Synthetic polynucleotide
gtctctcgttgt

SEQ ID NO: 193 - DNA, 12 bp, Artificial Sequence
  Description of Artificial Sequence Synthetic polynucleotide
taaacgcgtttg

SEQ ID NO: 194 - DNA, 12 bp, Artificial Sequence
  Description of Artificial Sequence Synthetic polynucleotide
gtgccacgaaac

SEQ ID NO: 195 - DNA, 12 bp, Artificial Sequence
  Description of Artificial Sequence Synthetic polynucleotide
caattgcaccat

SEQ ID NO: 196 - DNA, 12 bp, Artificial Sequence
  Description of Artificial Sequence Synthetic polynucleotide
cggtacggagtc

SEQ ID NO: 197 - DNA, 12 bp, Artificial Sequence
  Description of Artificial Sequence Synthetic polynucleotide
ttctctaggaac

SEQ ID NO: 198 - DNA, 12 bp, Artificial Sequence
  Description of Artificial Sequence Synthetic polynucleotide
aacgtcgaatta

SEQ ID NO: 199 - DNA, 12 bp, Artificial Sequence
  Description of Artificial Sequence Synthetic polynucleotide
ttgtccggaact

SEQ ID NO: 200 - DNA, 12 bp, Artificial Sequence
  Description of Artificial Sequence Synthetic polynucleotide
atgcctgtccaa

SEQ ID NO: 201 - DNA, 12 bp, Artificial Sequence
  Description of Artificial Sequence Synthetic polynucleotide
tgaagcaccaat

SEQ ID NO: 202 - DNA, 12 bp, Artificial Sequence
  Description of Artificial Sequence Synthetic polynucleotide
aaggatcctctt

SEQ ID NO: 203 - DNA, 12 bp, Artificial Sequence
  Description of Artificial Sequence Synthetic polynucleotide
cttcatgttaaa

SEQ ID NO: 204 - DNA, 12 bp, Artificial Sequence
  Description of Artificial Sequence Synthetic polynucleotide
ttgagccacctc

SEQ ID NO: 205 - DNA, 12 bp, Artificial Sequence
  Description of Artificial Sequence Synthetic polynucleotide
gcttatgttgac

SEQ ID NO: 206 - DNA, 12 bp, Artificial Sequence
  Description of Artificial Sequence Synthetic polynucleotide
cgacgatgggtt

SEQ ID NO: 207 - DNA, 12 bp, Artificial Sequence
  Description of Artificial Sequence Synthetic polynucleotide
cgttcgctatat

SEQ ID NO: 208 - DNA, 12 bp, Artificial Sequence
  Description of Artificial Sequence Synthetic polynucleotide
ccagttgctcag

SEQ ID NO: 209 - DNA, 12 bp, Artificial Sequence
  Description of Artificial Sequence Synthetic polynucleotide
cccggtctcaat

SEQ ID NO: 210 - DNA, 12 bp, Artificial Sequence
  Description of Artificial Sequence Synthetic polynucleotide
gtttggatcacg

SEQ ID NO: 211 - DNA, 12 bp, Artificial Sequence
  Description of Artificial Sequence Synthetic polynucleotide
ttgccttcgtac

SEQ ID NO: 212 - DNA, 12 bp, Artificial Sequence
  Description of Artificial Sequence Synthetic polynucleotide
agtagtgtccac

SEQ ID NO: 213 - DNA, 12 bp, Artificial Sequence
  Description of Artificial Sequence Synthetic polynucleotide
agaatttatcca

SEQ ID NO: 214 - DNA, 12 bp, Artificial Sequence
  Description of Artificial Sequence Synthetic polynucleotide
aatggatgcacg

SEQ ID NO: 215 - DNA, 12 bp, Artificial Sequence
  Description of Artificial Sequence Synthetic polynucleotide
atccaaggattg

SEQ ID NO: 216 - DNA, 12 bp, Artificial Sequence
  Description of Artificial Sequence Synthetic polynucleotide
tagtcgttagat

SEQ ID NO: 217 - DNA, 12 bp, Artificial Sequence
  Description of Artificial Sequence Synthetic polynucleotide
tgatactgagta

SEQ ID NO: 218 - DNA, 12 bp, Artificial Sequence
  Description of Artificial Sequence Synthetic polynucleotide
gcctagtaaaga

SEQ ID NO: 219 - DNA, 12 bp, Artificial Sequence
  Description of Artificial Sequence Synthetic polynucleotide
gcctacttatag

SEQ ID NO: 220 - DNA, 12 bp, Artificial Sequence
  Description of Artificial Sequence Synthetic polynucleotide
cctgccttggct

SEQ ID NO: 221 - DNA, 12 bp, Artificial Sequence
  Description of Artificial Sequence Synthetic polynucleotide
gatggacacaga

SEQ ID NO: 222 - DNA, 12 bp, Artificial Sequence
  Description of Artificial Sequence Synthetic polynucleotide
cgccgggttgga

SEQ ID NO: 223 - DNA, 12 bp, Artificial Sequence
  Description of Artificial Sequence Synthetic polynucleotide
gtctccaagatg

SEQ ID NO: 224 - DNA, 12 bp, Artificial Sequence
  Description of Artificial Sequence Synthetic polynucleotide
aaagcgtatcaa

SEQ ID NO: 225 - DNA, 12 bp, Artificial Sequence
  Description of Artificial Sequence Synthetic polynucleotide
gcaccgcagcgt

SEQ ID NO: 226 - DNA, 12 bp, Artificial Sequence
  Description of Artificial Sequence Synthetic polynucleotide
tgcgctgaatgc

SEQ ID NO: 227 - DNA, 12 bp, Artificial Sequence
  Description of Artificial Sequence Synthetic polynucleotide
ttgttcagagtc

SEQ ID NO: 228 - DNA, 12 bp, Artificial Sequence
  Description of Artificial Sequence Synthetic polynucleotide
gagaatgactga

SEQ ID NO: 229 - DNA, 12 bp, Artificial Sequence
  Description of Artificial Sequence Synthetic polynucleotide
aagcgaagcgac

SEQ ID NO: 230 - DNA, 12 bp, Artificial Sequence
  Description of Artificial Sequence Synthetic polynucleotide
agcgtgttgtaa

SEQ ID NO: 231 - DNA, 12 bp, Artificial Sequence
  Description of Artificial Sequence Synthetic polynucleotide
ccactatacctt

SEQ ID NO: 232 - DNA, 12 bp, Artificial Sequence
  Description of Artificial Sequence Synthetic polynucleotide
tgttaaactctg

SEQ ID NO: 233 - DNA, 12 bp, Artificial Sequence
  Description of Artificial Sequence Synthetic polynucleotide
atttgtagggcg

SEQ ID NO: 234 - DNA, 12 bp, Artificial Sequence
  Description of Artificial Sequence Synthetic polynucleotide
caggaatcatag

SEQ ID NO: 235 - DNA, 12 bp, Artificial Sequence
  Description of Artificial Sequence Synthetic polynucleotide
actggatagcat

SEQ ID NO: 236 - DNA, 12 bp, Artificial Sequence
  Description of Artificial Sequence Synthetic polynucleotide
gcacgtaacacg

SEQ ID NO: 237 - DNA, 12 bp, Artificial Sequence
  Description of Artificial Sequence Synthetic polynucleotide
aactgccacctg

SEQ ID NO: 238 - DNA, 12 bp, Artificial Sequence
  Description of Artificial Sequence Synthetic polynucleotide
agcaacctaagt

SEQ ID NO: 239 - DNA, 12 bp, Artificial Sequence
  Description of Artificial Sequence Synthetic polynucleotide
gactgctaacag

SEQ ID NO: 240 - DNA, 12 bp, Artificial Sequence
  Description of Artificial Sequence Synthetic polynucleotide
ttatagttcgct

SEQ ID NO: 241 - DNA, 12 bp, Artificial Sequence
  Description of Artificial Sequence Synthetic polynucleotide
tcgccacacaca

SEQ ID NO: 242 - DNA, 12 bp, Artificial Sequence
  Description of Artificial Sequence Synthetic polynucleotide
ataaccaccagt

SEQ ID NO: 243 - DNA, 12 bp, Artificial Sequence
  Description of Artificial Sequence Synthetic polynucleotide
gaatgggtccgc

SEQ ID NO: 244 - DNA, 12 bp, Artificial Sequence
  Description of Artificial Sequence Synthetic polynucleotide
gtgactatggct

SEQ ID NO: 245 - DNA, 12 bp, Artificial Sequence
  Description of Artificial Sequence Synthetic polynucleotide
tatagtcctgag

SEQ ID NO: 246 - DNA, 12 bp, Artificial Sequence
  Description of Artificial Sequence Synthetic polynucleotide
tgtacacatcac

SEQ ID NO: 247 - DNA, 12 bp, Artificial Sequence
  Description of Artificial Sequence Synthetic polynucleotide
taccttaatgac

SEQ ID NO: 248 - DNA, 12 bp, Artificial Sequence
  Description of Artificial Sequence Synthetic polynucleotide
gccacaaattaa

SEQ ID NO: 249 - DNA, 12 bp, Artificial Sequence
  Description of Artificial Sequence Synthetic polynucleotide
acgttcgggacg

SEQ ID NO: 250 - DNA, 12 bp, Artificial Sequence
  Description of Artificial Sequence Synthetic polynucleotide
ttaccgaaccca

SEQ ID NO: 251 - DNA, 12 bp, Artificial Sequence
  Description of Artificial Sequence Synthetic polynucleotide
gtgtcatattaa

SEQ ID NO: 252 - DNA, 12 bp, Artificial Sequence
  Description of Artificial Sequence Synthetic polynucleotide
tgaagcgacgag

SEQ ID NO: 253 - DNA, 12 bp, Artificial Sequence
  Description of Artificial Sequence Synthetic polynucleotide
atgccatgccgc

SEQ ID NO: 254 - DNA, 12 bp, Artificial Sequence
  Description of Artificial Sequence Synthetic polynucleotide
ttggcctcgatt

SEQ ID NO: 255 - DNA, 12 bp, Artificial Sequence
  Description of Artificial Sequence Synthetic polynucleotide
atcctcacttca

SEQ ID NO: 256 - DNA, 12 bp, Artificial Sequence
  Description of Artificial Sequence Synthetic polynucleotide
agattacttgga

SEQ ID NO: 257 - DNA, 12 bp, Artificial Sequence
  Description of Artificial Sequence Synthetic polynucleotide
taatatactggt

SEQ ID NO: 258 - DNA, 12 bp, Artificial Sequence
  Description of Artificial Sequence Synthetic polynucleotide
tcatgcggaaag

SEQ ID NO: 259 - DNA, 12 bp, Artificial Sequence
  Description of Artificial Sequence Synthetic polynucleotide
gaaggtccgcaa

SEQ ID NO: 260 - DNA, 12 bp, Artificial Sequence
  Description of Artificial Sequence Synthetic polynucleotide
acggtgatggaa

SEQ ID NO: 261 - DNA, 12 bp, Artificial Sequence
  Description of Artificial Sequence Synthetic polynucleotide
gacctaaccggt

SEQ ID NO: 262 - DNA, 12 bp, Artificial Sequence
  Description of Artificial Sequence Synthetic polynucleotide
ttagcgacagct

SEQ ID NO: 263 - DNA, 12 bp, Artificial Sequence
  Description of Artificial Sequence Synthetic polynucleotide
ttgccaccaatt

SEQ ID NO: 264 - DNA, 12 bp, Artificial Sequence
  Description of Artificial Sequence Synthetic polynucleotide
ctatcttgagat

SEQ ID NO: 265 - DNA, 12 bp, Artificial Sequence
  Description of Artificial Sequence Synthetic polynucleotide
atttcattccta

SEQ ID NO: 266 - DNA, 12 bp, Artificial Sequence
  Description of Artificial Sequence Synthetic polynucleotide
cgtggcgcatcg

SEQ ID NO: 267 - DNA, 12 bp, Artificial Sequence
  Description of Artificial Sequence Synthetic polynucleotide
ccattaattgtc

SEQ ID NO: 268 - DNA, 12 bp, Artificial Sequence
  Description of Artificial Sequence Synthetic polynucleotide
aagtttcttcat

SEQ ID NO: 269 - DNA, 12 bp, Artificial Sequence
  Description of Artificial Sequence Synthetic polynucleotide
tcgtagagaaca

SEQ ID NO: 270 - DNA, 12 bp, Artificial Sequence
  Description of Artificial Sequence Synthetic polynucleotide
acaaggttgatg

SEQ ID NO: 271 - DNA, 12 bp, Artificial Sequence
  Description of Artificial Sequence Synthetic polynucleotide
gtctttacatct

SEQ ID NO: 272 - DNA, 12 bp, Artificial Sequence
  Description of Artificial Sequence Synthetic polynucleotide
cttcgccgcgtg

SEQ ID NO: 273 - DNA, 12 bp, Artificial Sequence
  Description of Artificial Sequence Synthetic polynucleotide
taagactacaag

SEQ ID NO: 274 - DNA, 12 bp, Artificial Sequence
  Description of Artificial Sequence Synthetic polynucleotide
ctgtcgaagccg

SEQ ID NO: 275 - DNA, 12 bp, Artificial Sequence
  Description of Artificial Sequence Synthetic polynucleotide
taagcccaggaa

SEQ ID NO: 276 - DNA, 12 bp, Artificial Sequence
  Description of Artificial Sequence Synthetic polynucleotide
tattgccgtttc

SEQ ID NO: 277 - DNA, 12 bp, Artificial Sequence
  Description of Artificial Sequence Synthetic polynucleotide
taccgtctctaa

SEQ ID NO: 278 - DNA, 12 bp, Artificial Sequence
  Description of Artificial Sequence Synthetic polynucleotide
gaccattagagc

SEQ ID NO: 279 - DNA, 12 bp, Artificial Sequence
  Description of Artificial Sequence Synthetic polynucleotide
catcgacgctcg

SEQ ID NO: 280 - DNA, 12 bp, Artificial Sequence
  Description of Artificial Sequence Synthetic polynucleotide
ttgcccgactca

SEQ ID NO: 281 - DNA, 12 bp, Artificial Sequence
  Description of Artificial Sequence Synthetic polynucleotide
acgtacgctagt

SEQ ID NO: 282 - DNA, 12 bp, Artificial Sequence
  Description of Artificial Sequence Synthetic polynucleotide
catagacaactt

SEQ ID NO: 283 - DNA, 12 bp, Artificial Sequence
  Description of Artificial Sequence Synthetic polynucleotide
tgaagatagtcg

SEQ ID NO: 284 - DNA, 12 bp, Artificial Sequence
  Description of Artificial Sequence Synthetic polynucleotide
taagataagatg

SEQ ID NO: 285 - DNA, 12 bp, Artificial Sequence
  Description of Artificial Sequence Synthetic polynucleotide
aatagcgagctc

SEQ ID NO: 286 - DNA, 12 bp, Artificial Sequence
  Description of Artificial Sequence Synthetic polynucleotide
accggcgatggt

SEQ ID NO: 287 - DNA, 12 bp, Artificial Sequence
  Description of Artificial Sequence Synthetic polynucleotide
agttctggcaaa

SEQ ID NO: 288 - DNA, 12 bp, Artificial Sequence
  Description of Artificial Sequence Synthetic polynucleotide
tccctgcgcgac

SEQ ID NO: 289 - DNA, 12 bp, Artificial Sequence
  Description of Artificial Sequence Synthetic polynucleotide
aatatagcctct

SEQ ID NO: 290 - DNA, 12 bp, Artificial Sequence
  Description of Artificial Sequence Synthetic polynucleotide
tttccgctacag

SEQ ID NO: 291 - DNA, 12 bp, Artificial Sequence
  Description of Artificial Sequence Synthetic polynucleotide
gcttagatccat

SEQ ID NO: 292 - DNA, 12 bp, Artificial Sequence
  Description of Artificial Sequence Synthetic polynucleotide
gctctcgaacgt

SEQ ID NO: 293 - DNA, 12 bp, Artificial Sequence
  Description of Artificial Sequence Synthetic polynucleotide
gataccgattac

SEQ ID NO: 294 - DNA, 12 bp, Artificial Sequence
  Description of Artificial Sequence Synthetic polynucleotide
acgaactcctgt

SEQ ID NO: 295 - DNA, 12 bp, Artificial Sequence
  Description of Artificial Sequence Synthetic polynucleotide
ataaacgtcggt

SEQ ID NO: 296 - DNA, 12 bp, Artificial Sequence
  Description of Artificial Sequence Synthetic polynucleotide
gacgtactccaa

SEQ ID NO: 297 - DNA, 12 bp, Artificial Sequence
  Description of Artificial Sequence Synthetic polynucleotide
gcaagctcgaga

SEQ ID NO: 298 - DNA, 12 bp, Artificial Sequence
  Description of Artificial Sequence Synthetic polynucleotide
tcgtgcttctta

SEQ ID NO: 299 - DNA, 12 bp, Artificial Sequence
  Description of Artificial Sequence Synthetic polynucleotide
atccgaaccaag

SEQ ID NO: 300 - DNA, 12 bp, Artificial Sequence
  Description of Artificial Sequence Synthetic polynucleotide
gaacatcattga

SEQ ID NO: 301 - DNA, 12 bp, Artificial Sequence
  Description of Artificial Sequence Synthetic polynucleotide
taagccctagtt

SEQ ID NO: 302 - DNA, 12 bp, Artificial Sequence
  Description of Artificial Sequence Synthetic polynucleotide
tgaccacggttc

SEQ ID NO: 303 - DNA, 12 bp, Artificial Sequence
  Description of Artificial Sequence Synthetic polynucleotide
ccaatggagaag

SEQ ID NO: 304 - DNA, 12 bp, Artificial Sequence
  Description of Artificial Sequence Synthetic polynucleotide
gttgggccagca

SEQ ID NO: 305 - DNA, 12 bp, Artificial Sequence
  Description of Artificial Sequence Synthetic polynucleotide
actcgatttacg

SEQ ID NO: 306 - DNA, 12 bp, Artificial Sequence
  Description of Artificial Sequence Synthetic polynucleotide
agccgttgctgt

SEQ ID NO: 307 - DNA, 12 bp, Artificial Sequence
  Description of Artificial Sequence Synthetic polynucleotide
gtcttcgaaaga

SEQ ID NO: 308 - DNA, 12 bp, Artificial Sequence
  Description of Artificial Sequence Synthetic polynucleotide
acgtctggtgat

SEQ ID NO: 309 - DNA, 12 bp, Artificial Sequence
  Description of Artificial Sequence Synthetic polynucleotide
gaatggcaaact

SEQ ID NO: 310 - DNA, 12 bp, Artificial Sequence
  Description of Artificial Sequence Synthetic polynucleotide
tatgagagttcg

SEQ ID NO: 311 - DNA, 12 bp, Artificial Sequence
  Description of Artificial Sequence Synthetic polynucleotide
catctggagctg

SEQ ID NO: 312 - DNA, 12 bp, Artificial Sequence
  Description of Artificial Sequence Synthetic polynucleotide
ccagcgacttct

SEQ ID NO: 313 - DNA, 12 bp, Artificial Sequence
  Description of Artificial Sequence Synthetic polynucleotide
actcaatcgact

SEQ ID NO: 314 - DNA, 12 bp, Artificial Sequence
  Description of Artificial Sequence Synthetic polynucleotide
tccagctaacct

SEQ ID NO: 315 - DNA, 12 bp, Artificial Sequence
  Description of Artificial Sequence Synthetic polynucleotide
acttaacgggag

SEQ ID NO: 316 - DNA, 12 bp, Artificial Sequence
  Description of Artificial Sequence Synthetic polynucleotide
gagcttctggca

SEQ ID NO: 317 - DNA, 12 bp, Artificial Sequence
  Description of Artificial Sequence Synthetic polynucleotide
tcatacacgttc

SEQ ID NO: 318 - DNA, 12 bp, Artificial Sequence
  Description of Artificial Sequence Synthetic polynucleotide
cctgtgattcca

SEQ ID NO: 319 - DNA, 12 bp, Artificial Sequence
  Description of Artificial Sequence Synthetic polynucleotide
gctcttgatgtc

SEQ ID NO: 320 - DNA, 12 bp, Artificial Sequence
  Description of Artificial Sequence Synthetic polynucleotide
ctgattcccgga

SEQ ID NO: 321 - DNA, 12 bp, Artificial Sequence
  Description of Artificial Sequence Synthetic polynucleotide
aatcatagcaac

SEQ ID NO: 322 - DNA, 12 bp, Artificial Sequence
  Description of Artificial Sequence Synthetic polynucleotide
aagcgtaaaccg

SEQ ID NO: 323 - DNA, 12 bp, Artificial Sequence
  Description of Artificial Sequence Synthetic polynucleotide
ctcttgaaacct

SEQ ID NO: 324 - DNA, 12 bp, Artificial Sequence
  Description of Artificial Sequence Synthetic polynucleotide
ctatcaggccca

SEQ ID NO: 325 - DNA, 12 bp, Artificial Sequence
  Description of Artificial Sequence Synthetic polynucleotide
aagtgtaacaac

SEQ ID NO: 326 - DNA, 12 bp, Artificial Sequence
  Description of Artificial Sequence Synthetic polynucleotide
acattactactg

SEQ ID NO: 327 - DNA, 12 bp, Artificial Sequence
  Description of Artificial Sequence Synthetic polynucleotide
tcaagtttatgc

SEQ ID NO: 328 - DNA, 12 bp, Artificial Sequence
  Description of Artificial Sequence Synthetic polynucleotide
tcgtgtacacac

SEQ ID NO: 329 - DNA, 12 bp, Artificial Sequence
  Description of Artificial Sequence Synthetic polynucleotide
gatagctctgga

SEQ ID NO: 330 - DNA, 12 bp, Artificial Sequence
  Description of Artificial Sequence Synthetic polynucleotide
caaagttaaagc

SEQ ID NO: 331 - DNA, 12 bp, Artificial Sequence
  Description of Artificial Sequence Synthetic polynucleotide
tgtatactgaac

SEQ ID NO: 332 - DNA, 12 bp, Artificial Sequence
  Description of Artificial Sequence Synthetic polynucleotide
aacaaacggtct

SEQ ID NO: 333 - DNA, 12 bp, Artificial Sequence
  Description of Artificial Sequence Synthetic polynucleotide
tccgtgcaggtt

SEQ ID NO: 334 - DNA, 12 bp, Artificial Sequence
  Description of Artificial Sequence Synthetic polynucleotide
agcaagttaggc

SEQ ID NO: 335 - DNA, 12 bp, Artificial Sequence
  Description of Artificial Sequence Synthetic polynucleotide
ctagtgaactat

SEQ ID NO: 336 - DNA, 12 bp, Artificial Sequence
  Description of Artificial Sequence Synthetic polynucleotide
atgtgaatgaca

SEQ ID NO: 337 - DNA, 12 bp, Artificial Sequence
  Description of Artificial Sequence Synthetic polynucleotide
acgacgcgcgta

SEQ ID NO: 338 - DNA, 12 bp, Artificial Sequence
  Description of Artificial Sequence Synthetic polynucleotide
acgtgcaggcga

SEQ ID NO: 339 - DNA, 12 bp, Artificial Sequence
  Description of Artificial Sequence Synthetic polynucleotide
caagtatgcaat

SEQ ID NO: 340 - DNA, 12 bp, Artificial Sequence
  Description of Artificial Sequence Synthetic polynucleotide
gtactcccgtta

SEQ ID NO: 341 - DNA, 12 bp, Artificial Sequence
  Description of Artificial Sequence Synthetic polynucleotide
aatacaaccttc

SEQ ID NO: 342 - DNA, 12 bp, Artificial Sequence
  Description of Artificial Sequence Synthetic polynucleotide
acccattcccgc

SEQ ID NO: 343 - DNA, 12 bp, Artificial Sequence
  Description of Artificial Sequence Synthetic polynucleotide
ttagctaacaac

SEQ ID NO: 344 - DNA, 12 bp, Artificial Sequence
  Description of Artificial Sequence Synthetic polynucleotide
tgccgcttggac

SEQ ID NO: 345 - DNA, 12 bp, Artificial Sequence
  Description of Artificial Sequence Synthetic polynucleotide
tgataacgtagt

SEQ ID NO: 346 - DNA, 12 bp, Artificial Sequence
  Description of Artificial Sequence Synthetic polynucleotide
tgcctcgcctaa

SEQ ID NO: 347 - DNA, 12 bp, Artificial Sequence
  Description of Artificial Sequence Synthetic polynucleotide
tacttgcggctc

SEQ ID NO: 348 - DNA, 12 bp, Artificial Sequence
  Description of Artificial Sequence Synthetic polynucleotide
tcgttgtcagga

SEQ ID NO: 349 - DNA, 12 bp, Artificial Sequence
  Description of Artificial Sequence Synthetic polynucleotide
atctcaagcgtt

SEQ ID NO: 350 - DNA, 12 bp, Artificial Sequence
  Description of Artificial Sequence Synthetic polynucleotide
ccacctggtctt

SEQ ID NO: 351 - DNA, 12 bp, Artificial Sequence
  Description of Artificial Sequence Synthetic polynucleotide
actaaaccacac

SEQ ID NO: 352 - DNA, 12 bp, Artificial Sequence
  Description of Artificial Sequence Synthetic polynucleotide
ttcgggctcgag

SEQ ID NO: 353 - DNA, 12 bp, Artificial Sequence
  Description of Artificial Sequence Synthetic polynucleotide
aactcccttcgt

SEQ ID NO: 354 - DNA, 12 bp, Artificial Sequence
  Description of Artificial Sequence Synthetic polynucleotide
gttaatagaggt

SEQ ID NO: 355 - DNA, 12 bp, Artificial Sequence
  Description of Artificial Sequence Synthetic polynucleotide
tcggacttataa

SEQ ID NO: 356 - DNA, 12 bp, Artificial Sequence
  Description of Artificial Sequence Synthetic polynucleotide
ccgtaagtatgt

SEQ ID NO: 357 - DNA, 12 bp, Artificial Sequence
  Description of Artificial Sequence Synthetic polynucleotide
cacccagagtta

SEQ ID NO: 358 - DNA, 12 bp, Artificial Sequence
  Description of Artificial Sequence Synthetic polynucleotide
ctatcctaggac

SEQ ID NO: 359 - DNA, 12 bp, Artificial Sequence
  Description of Artificial Sequence Synthetic polynucleotide
tcttgctcggtg

SEQ ID NO: 360 - DNA, 12 bp, Artificial Sequence
  Description of Artificial Sequence Synthetic polynucleotide
gcggagctttgc

SEQ ID NO: 361 - DNA, 24 bp, Artificial Sequence
  Description of Artificial Sequence Synthetic polynucleotide
caagcagaagacggcatacgagat

SEQ ID NO: 362 - DNA, 34 bp, Artificial Sequence
  Description of Artificial Sequence Synthetic polynucleotide
gtgactggagttcagacgtgtgctcttccgatct

SEQ ID NO: 363 - DNA, 12 bp, Artificial Sequence
  Description of Artificial Sequence Synthetic polynucleotide
aatgcttcttgt

SEQ ID NO: 364 - DNA, 90 bp, Artificial Sequence
  Description of Artificial Sequence Synthetic polynucleotide
  modified_base (71)..(90): a, c, t, g, unknown or other
  See specification as filed for detailed description of substitutions and preferred embodiments
caagcagaagacggcatacgagataatgcttcttgtgtgactggagttcagacgtgtgctcttccgatctnnnnnnnnnnnnnnnnnnnn

SEQ ID NO: 365 - DNA, 54 bp, Artificial Sequence
  Description of Artificial Sequence Synthetic polynucleotide
  modified_base (35)..(54): a, c, t, g, unknown or other
  See specification as filed for detailed description of substitutions and preferred embodiments
gtgactggagttcagacgtgtgctcttccgatctnnnnnnnnnnnnnnnnnnnn

SEQ ID NO: 366 - DNA, 12 bp, Artificial Sequence
  Description of Artificial Sequence Synthetic polynucleotide
gcttattagtta

SEQ ID NO: 367 - DNA, 12 bp, Artificial Sequence
  Description of Artificial Sequence Synthetic polynucleotide
tcggccgtctcg

SEQ ID NO: 368 - DNA, 12 bp, Artificial Sequence
  Description of Artificial Sequence Synthetic polynucleotide
gcgattgcttag

SEQ ID NO: 369 - DNA, 12 bp, Artificial Sequence
  Description of Artificial Sequence Synthetic polynucleotide
tggagcgaacat

SEQ ID NO: 370 - DNA, 12 bp, Artificial Sequence
  Description of Artificial Sequence Synthetic polynucleotide
gcgctggacctt

SEQ ID NO: 371 - DNA, 12 bp, Artificial Sequence
  Description of Artificial Sequence Synthetic polynucleotide
gctgatcaatga

SEQ ID NO: 372 - DNA, 12 bp, Artificial Sequence
  Description of Artificial Sequence Synthetic polynucleotide
ctgaagctctaa

SEQ ID NO: 373 - DNA, 12 bp, Artificial Sequence
  Description of Artificial Sequence Synthetic polynucleotide
attgtaagggcc

SEQ ID NO: 374 - DNA, 12 bp, Artificial Sequence
  Description of Artificial Sequence Synthetic polynucleotide
gtttaggttgcc

SEQ ID NO: 375 - DNA, 12 bp, Artificial Sequence
  Description of Artificial Sequence Synthetic polynucleotide
cagtggagcacc

SEQ ID NO: 376 - DNA, 12 bp, Artificial Sequence
  Description of Artificial Sequence Synthetic polynucleotide
tctacctagcgc

SEQ ID NO: 377 - DNA, 12 bp, Artificial Sequence
  Description of Artificial Sequence Synthetic polynucleotide
gtagcaagtatg

SEQ ID NO: 378 - DNA, 12 bp, Artificial Sequence
  Description of Artificial Sequence Synthetic polynucleotide
tgagggtgcctc

SEQ ID NO: 379 - DNA, 12 bp, Artificial Sequence
  Description of Artificial Sequence Synthetic polynucleotide
taacccagaacg

SEQ ID NO: 380 - DNA, 12 bp, Artificial Sequence
  Description of Artificial Sequence Synthetic polynucleotide
cattccagtgtt

SEQ ID NO: 381 - DNA, 12 bp, Artificial Sequence
  Description of Artificial Sequence Synthetic polynucleotide
gtgatagccata

SEQ ID NO: 382 - DNA, 12 bp, Artificial Sequence
  Description of Artificial Sequence Synthetic polynucleotide
ctagcgactagt

SEQ ID NO: 383 - DNA, 12 bp, Artificial Sequence
  Description of Artificial Sequence Synthetic polynucleotide
ctcttcggaccc

SEQ ID NO: 384 - DNA, 12 bp, Artificial Sequence
  Description of Artificial Sequence Synthetic polynucleotide
ataacgcgaccc

SEQ ID NO: 385 - DNA, 12 bp, Artificial Sequence
  Description of Artificial Sequence Synthetic polynucleotide
ataaagatcatc

SEQ ID NO: 386 - DNA, 12 bp, Artificial Sequence
  Description of Artificial Sequence Synthetic polynucleotide
catcgggaaggc

SEQ ID NO: 387 - DNA, 12 bp, Artificial Sequence
  Description of Artificial Sequence Synthetic polynucleotide
gtgtcggcaggt

SEQ ID NO: 388 - DNA, 12 bp, Artificial Sequence
  Description of Artificial Sequence Synthetic polynucleotide
acacggaacgtg

SEQ ID NO: 389 - DNA, 12 bp, Artificial Sequence
  Description of Artificial Sequence Synthetic polynucleotide
gcagttctgagc

SEQ ID NO: 390 - DNA, 12 bp, Artificial Sequence
  Description of Artificial Sequence Synthetic polynucleotide
gagttgcgctag

SEQ ID NO: 391 - DNA, 12 bp, Artificial Sequence
  Description of Artificial Sequence Synthetic polynucleotide
aggacgtagtat

SEQ ID NO: 392 - DNA, 12 bp, Artificial Sequence
  Description of Artificial Sequence Synthetic polynucleotide
taccaatagaca

SEQ ID NO: 393 - DNA, 12 bp, Artificial Sequence
  Description of Artificial Sequence Synthetic polynucleotide
aaagtctgtatg

SEQ ID NO: 394 - DNA, 12 bp, Artificial Sequence
  Description of Artificial Sequence Synthetic polynucleotide
aaatttcgactc

SEQ ID NO: 395 - DNA, 12 bp, Artificial Sequence
  Description of Artificial Sequence Synthetic polynucleotide
ttggatcttaag

SEQ ID NO: 396 - DNA, 12 bp, Artificial Sequence
  Description of Artificial Sequence Synthetic polynucleotide
tcaccagcgcgt

SEQ ID NO: 397 - DNA, 12 bp, Artificial Sequence
  Description of Artificial Sequence Synthetic polynucleotide
acgccttccaca

SEQ ID NO: 398 - DNA, 12 bp, Artificial Sequence
  Description of Artificial Sequence Synthetic polynucleotide
taggactcatat

SEQ ID NO: 399 - DNA, 12 bp, Artificial Sequence
  Description of Artificial Sequence Synthetic polynucleotide
gcaaacctaact

SEQ ID NO: 400 - DNA, 12 bp, Artificial Sequence
  Description of Artificial Sequence Synthetic polynucleotide
tgtaagtggata

SEQ ID NO: 401 - DNA, 12 bp, Artificial Sequence
  Description of Artificial Sequence Synthetic polynucleotide
gcagcttaacct

SEQ ID NO: 402 - DNA, 12 bp, Artificial Sequence
  Description of Artificial Sequence Synthetic polynucleotide
actagtcgctac

SEQ ID NO: 403 - DNA, 12 bp, Artificial Sequence
  Description of Artificial Sequence Synthetic polynucleotide
tgttaaatgggc

SEQ ID NO: 404 - DNA, 12 bp, Artificial Sequence
  Description of Artificial Sequence Synthetic polynucleotide
gacccaatcatc

SEQ ID NO: 405 - DNA, 12 bp, Artificial Sequence
  Description of Artificial Sequence Synthetic polynucleotide
aggagcctacag

SEQ ID NO: 406 - DNA, 12 bp, Artificial Sequence
  Description of Artificial Sequence Synthetic polynucleotide
gctagggagacg

SEQ ID NO: 407 - DNA, 12 bp, Artificial Sequence
  Description of Artificial Sequence Synthetic polynucleotide
tgttcctccacc

SEQ ID NO: 408 - DNA, 12 bp, Artificial Sequence
  Description of Artificial Sequence Synthetic polynucleotide
cttgaaggttca

SEQ ID NO: 409 - DNA, 12 bp, Artificial Sequence
  Description of Artificial Sequence Synthetic polynucleotide
gtcgacgaacct

SEQ ID NO: 410 - DNA, 12 bp, Artificial Sequence
  Description of Artificial Sequence Synthetic polynucleotide
cgcttccactta

SEQ ID NO: 411 - DNA, 12 bp, Artificial Sequence
  Description of Artificial Sequence Synthetic polynucleotide
caacctttgctc

SEQ ID NO: 412 - DNA, 12 bp, Artificial Sequence
  Description of Artificial Sequence Synthetic polynucleotide
acatgggagcct

SEQ ID NO: 413 - DNA, 12 bp, Artificial Sequence
  Description of Artificial Sequence Synthetic polynucleotide
tccgatataatg

SEQ ID NO: 414 - DNA, 12 bp, Artificial Sequence
  Description of Artificial Sequence Synthetic polynucleotide
tattggataatg

SEQ ID NO: 415 - DNA, 12 bp, Artificial Sequence
  Description of Artificial Sequence Synthetic polynucleotide
gtattcacgggt

SEQ ID NO: 416 - DNA, 12 bp, Artificial Sequence
  Description of Artificial Sequence Synthetic polynucleotide
tttatgttacgc

SEQ ID NO: 417 - DNA, 12 bp, Artificial Sequence
  Description of Artificial Sequence Synthetic polynucleotide
agtgatagatcc

SEQ ID NO: 418 - DNA, 12 bp, Artificial Sequence
  Description of Artificial Sequence Synthetic polynucleotide
gcattaggtact

SEQ ID NO: 419 - DNA, 12 bp, Artificial Sequence
  Description of Artificial Sequence Synthetic polynucleotide
tcgaagcactca

SEQ ID NO: 420 - DNA, 12 bp, Artificial Sequence
  Description of Artificial Sequence Synthetic polynucleotide
aaaccgcttaac

SEQ ID NO: 421 - DNA, 12 bp, Artificial Sequence
  Description of Artificial Sequence Synthetic polynucleotide
catatcttacaa

SEQ ID NO: 422 - DNA, 12 bp, Artificial Sequence
  Description of Artificial Sequence Synthetic polynucleotide
aaagttcacaac

SEQ ID NO: 423 - DNA, 12 bp, Artificial Sequence
  Description of Artificial Sequence Synthetic polynucleotide
ctctgtgagaga

SEQ ID NO: 424 - DNA, 12 bp, Artificial Sequence
  Description of Artificial Sequence Synthetic polynucleotide
ctcggtttactt

SEQ ID NO: 425 - DNA, 12 bp, Artificial Sequence
  Description of Artificial Sequence Synthetic polynucleotide
gccatggttgcg

SEQ ID NO: 426 - DNA, 12 bp, Artificial Sequence
  Description of Artificial Sequence Synthetic polynucleotide
agtggcagtcac

SEQ ID NO: 427 - DNA, 12 bp, Artificial Sequence
  Description of Artificial Sequence Synthetic polynucleotide
taagtcactggc

SEQ ID NO: 428 - DNA, 12 bp, Artificial Sequence
  Description of Artificial Sequence Synthetic polynucleotide
gatagagtctcc

SEQ ID NO: 429 - DNA, 12 bp, Artificial Sequence
  Description of Artificial Sequence Synthetic polynucleotide
gttactacgcta

SEQ ID NO: 430 - DNA, 12 bp, Artificial Sequence
  Description of Artificial Sequence Synthetic polynucleotide
cgcgactttgac

SEQ ID NO: 431 - DNA, 12 bp, Artificial Sequence
  Description of Artificial Sequence Synthetic polynucleotide
ttatattggcta

SEQ ID NO: 432 - DNA, 12 bp, Artificial Sequence
  Description of Artificial Sequence Synthetic polynucleotide
actattacgtgt

SEQ ID NO: 433 - DNA, 12 bp, Artificial Sequence
  Description of Artificial Sequence Synthetic polynucleotide
tcgcagagggct

SEQ ID NO: 434 - DNA, 12 bp, Artificial Sequence
  Description of Artificial Sequence Synthetic polynucleotide
tttatttcttcg

SEQ ID NO: 435 - DNA, 12 bp, Artificial Sequence
  Description of Artificial Sequence Synthetic polynucleotide
tagcctttatgc

SEQ ID NO: 436 - DNA, 12 bp, Artificial Sequence
  Description of Artificial Sequence Synthetic polynucleotide
tgtctgtaagga

SEQ ID NO: 437 - DNA, 12 bp, Artificial Sequence
  Description of Artificial Sequence Synthetic polynucleotide
tcagttcgcatt

SEQ ID NO: 438 - DNA, 12 bp, Artificial Sequence
  Description of Artificial Sequence Synthetic polynucleotide
tgtggaaagagc

SEQ ID NO: 439 - DNA, 12 bp, Artificial Sequence
  Description of Artificial Sequence Synthetic polynucleotide
gtcgaacttaat

SEQ ID NO: 440 - DNA, 12 bp, Artificial Sequence
  Description of Artificial Sequence Synthetic polynucleotide
caactggcaggt

SEQ ID NO: 441 - DNA, 12 bp, Artificial Sequence
  Description of Artificial Sequence Synthetic polynucleotide
tgggtggcaatg

SEQ ID NO: 442 - DNA, 12 bp, Artificial Sequence
  Description of Artificial Sequence Synthetic polynucleotide
cggtcggcacta

SEQ ID NO: 443 - DNA, 12 bp, Artificial Sequence
  Description of Artificial Sequence Synthetic polynucleotide
tgcgcttacaat

SEQ ID NO: 444 - DNA, 12 bp, Artificial Sequence
  Description of Artificial Sequence Synthetic polynucleotide
cgtattggtcta

SEQ ID NO: 445 - DNA, 12 bp, Artificial Sequence
  Description of Artificial Sequence Synthetic polynucleotide
cgacccatcgcg

SEQ ID NO: 446 - DNA, 12 bp, Artificial Sequence
  Description of Artificial Sequence Synthetic polynucleotide
ttgagggtgcga

SEQ ID NO: 447 - DNA, 12 bp, Artificial Sequence
  Description of Artificial Sequence Synthetic polynucleotide
atcgaggaagat

SEQ ID NO: 448 - DNA, 12 bp, Artificial Sequence
  Description of Artificial Sequence Synthetic polynucleotide
ctcgacatgctt

SEQ ID NO: 449 - DNA, 12 bp, Artificial Sequence
  Description of Artificial Sequence Synthetic polynucleotide
agagcaggattt

SEQ ID NO: 450 - DNA, 12 bp, Artificial Sequence
  Description of Artificial Sequence Synthetic polynucleotide
agcaagacggtt

SEQ ID NO: 451 - DNA, 12 bp, Artificial Sequence
  Description of Artificial Sequence Synthetic polynucleotide
ttactcagtaca

SEQ ID NO: 452 - DNA, 12 bp, Artificial Sequence
  Description of Artificial Sequence Synthetic polynucleotide
agtagcttgacc

SEQ ID NO: 453 - DNA, 12 bp, Artificial Sequence
  Description of Artificial Sequence Synthetic polynucleotide
agctgcatgcct

SEQ ID NO: 454 - DNA, 12 bp, Artificial Sequence
  Description of Artificial Sequence Synthetic polynucleotide
ctacagtcggct

SEQ ID NO: 455 - DNA, 12 bp, Artificial Sequence
  Description of Artificial Sequence Synthetic polynucleotide
tttcagggttgc

SEQ ID NO: 456 - DNA, 12 bp, Artificial Sequence
  Description of Artificial Sequence Synthetic polynucleotide
ataggtctacgt

SEQ ID NO: 457 - DNA, 12 bp, Artificial Sequence
  Description of Artificial Sequence Synthetic polynucleotide
ttccaaaccggt

SEQ ID NO: 458 - DNA, 12 bp, Artificial Sequence
  Description of Artificial Sequence Synthetic polynucleotide
gttccgtcgtct

SEQ ID NO: 459 - DNA, 12 bp, Artificial Sequence
  Description of Artificial Sequence Synthetic polynucleotide
tcaataggactc

SEQ ID NO: 460 - DNA, 12 bp, Artificial Sequence
  Description of Artificial Sequence Synthetic polynucleotide
atttgaagccca

SEQ ID NO: 461 - DNA, 12 bp, Artificial Sequence
  Description of Artificial Sequence Synthetic polynucleotide
atcgtcaaatga

SEQ ID NO: 462 - DNA, 12 bp, Artificial Sequence
  Description of Artificial Sequence Synthetic polynucleotide
tggatacccgtt

SEQ ID NO: 463 - DNA, 12 bp, Artificial Sequence
  Description of Artificial Sequence Synthetic polynucleotide
gattgatggtga

SEQ ID NO: 464 - DNA, 12 bp, Artificial Sequence
  Description of Artificial Sequence Synthetic polynucleotide
gccgcaaataac

SEQ ID NO: 465 - DNA, 12 bp, Artificial Sequence
  Description of Artificial Sequence Synthetic polynucleotide
taaatcctcacg

SEQ ID NO: 466 - DNA, 12 bp, Artificial Sequence
  Description of Artificial Sequence Synthetic polynucleotide
agtatacagacc

SEQ ID NO: 467 - DNA, 12 bp, Artificial Sequence
  Description of Artificial Sequence Synthetic polynucleotide
tcggatcatgaa

SEQ ID NO: 468 - DNA, 12 bp, Artificial Sequence
  Description of Artificial Sequence Synthetic polynucleotide
actgcccattac

SEQ ID NO: 469 - DNA, 12 bp, Artificial Sequence
  Description of Artificial Sequence Synthetic polynucleotide
taagagttggca

SEQ ID NO: 470 - DNA, 12 bp, Artificial Sequence
  Description of Artificial Sequence Synthetic polynucleotide
taagactccatc

SEQ ID NO: 471 - DNA, 12 bp, Artificial Sequence
  Description of Artificial Sequence Synthetic polynucleotide
tgccacatgttt

SEQ ID NO: 472 - DNA, 12 bp, Artificial Sequence
  Description of Artificial Sequence Synthetic polynucleotide
aatggacaagcc

SEQ ID NO: 473 - DNA, 12 bp, Artificial Sequence
  Description of Artificial Sequence Synthetic polynucleotide
atcctccaggca

SEQ ID NO: 474 - DNA, 12 bp, Artificial Sequence
  Description of Artificial Sequence Synthetic polynucleotide
gactgaatagat

SEQ ID NO: 475 - DNA, 12 bp, Artificial Sequence
  Description of Artificial Sequence Synthetic polynucleotide
attcggctttaa

SEQ ID NO: 476 - DNA, 12 bp, Artificial Sequence
  Description of Artificial Sequence Synthetic polynucleotide
gaagtttgttaa

SEQ ID NO: 477 - DNA, 12 bp, Artificial Sequence
  Description of Artificial Sequence Synthetic polynucleotide
gaagtcgaggta

SEQ ID NO: 478 - DNA, 12 bp, Artificial Sequence
  Description of Artificial Sequence Synthetic polynucleotide
gaccattgtcga

SEQ ID NO: 479 - DNA, 12 bp, Artificial Sequence
  Description of Artificial Sequence Synthetic polynucleotide
gccctccaatcc

SEQ ID NO: 480 - DNA, 12 bp, Artificial Sequence
  Description of Artificial Sequence Synthetic polynucleotide
gtcgtgtcggga

SEQ ID NO: 481 - DNA, 12 bp, Artificial Sequence
  Description of Artificial Sequence Synthetic polynucleotide
gcgtttcgtaca

SEQ ID NO: 482 - DNA, 12 bp, Artificial Sequence
  Description of Artificial Sequence Synthetic polynucleotide
tatagttgtcat

SEQ ID NO: 483 - DNA, 12 bp, Artificial Sequence
  Description of Artificial Sequence Synthetic polynucleotide
atccacgcgtgc

SEQ ID NO: 484 - DNA, 12 bp, Artificial Sequence
  Description of Artificial Sequence Synthetic polynucleotide
cgatttctagag

SEQ ID NO: 485 - DNA, 12 bp, Artificial Sequence
  Description of Artificial Sequence Synthetic polynucleotide
aacgcgacgtaa

SEQ ID NO: 486 - DNA, 12 bp, Artificial Sequence
  Description of Artificial Sequence Synthetic polynucleotide
gctcaaccgacc

SEQ ID NO: 487 - DNA, 12 bp, Artificial Sequence
  Description of Artificial Sequence Synthetic polynucleotide
tactggtaacat

SEQ ID NO: 488 - DNA, 12 bp, Artificial Sequence
  Description of Artificial Sequence Synthetic polynucleotide
acatcagagaat

SEQ ID NO: 489 - DNA, 12 bp, Artificial Sequence
  Description of Artificial Sequence Synthetic polynucleotide
ctagtagaattc

SEQ ID NO: 490 - DNA, 12 bp, Artificial Sequence
  Description of Artificial Sequence Synthetic polynucleotide
agcgcggtactg

SEQ ID NO: 491 - DNA, 12 bp, Artificial Sequence
  Description of Artificial Sequence Synthetic polynucleotide
ttgagatatgca

SEQ ID NO: 492 - DNA, 12 bp, Artificial Sequence
  Description of Artificial Sequence Synthetic polynucleotide
ttacccgtttaa

SEQ ID NO: 493 - DNA, 12 bp, Artificial Sequence
  Description of Artificial Sequence Synthetic polynucleotide
actagtctcatt

SEQ ID NO: 494 - DNA, 12 bp, Artificial Sequence
  Description of Artificial Sequence Synthetic polynucleotide
ttgttctgttga

SEQ ID NO: 495 - DNA, 12 bp, Artificial Sequence
  Description of Artificial Sequence Synthetic polynucleotide
cagtaaggtgag

SEQ ID NO: 496 - DNA, 12 bp, Artificial Sequence
  Description of Artificial Sequence Synthetic polynucleotide
aacgttcgcctg

SEQ ID NO: 497 - DNA, 12 bp, Artificial Sequence
  Description of Artificial Sequence Synthetic polynucleotide
tctggtgcattg

SEQ ID NO: 498 - DNA, 12 bp, Artificial Sequence
  Description of Artificial Sequence Synthetic polynucleotide
ttcggtgtctag

SEQ ID NO: 499 - DNA, 12 bp, Artificial Sequence
  Description of Artificial Sequence Synthetic polynucleotide
atgtctgaaatc

SEQ ID NO: 500 - DNA, 12 bp, Artificial Sequence
  Description of Artificial Sequence Synthetic polynucleotide
tctggccatgct

SEQ ID NO: 501 - DNA, 12 bp, Artificial Sequence
  Description of Artificial Sequence Synthetic polynucleotide
taatgtcttgag

SEQ ID NO: 502 - DNA, 12 bp, Artificial Sequence
  Description of Artificial Sequence Synthetic polynucleotide
cagactcacccg

SEQ ID NO: 503 - DNA, 12 bp, Artificial Sequence
  Description of Artificial Sequence Synthetic polynucleotide
ctactgatagga

SEQ ID NO: 504 - DNA, 12 bp, Artificial Sequence
  Description of Artificial Sequence Synthetic polynucleotide
acgggctcgtta

SEQ ID NO: 505 - DNA, 12 bp, Artificial Sequence
  Description of Artificial Sequence Synthetic polynucleotide
cattgcagacac

SEQ ID NO: 506 - DNA, 12 bp, Artificial Sequence
  Description of Artificial Sequence Synthetic polynucleotide
gaataagggacc

SEQ ID NO: 507 - DNA, 12 bp, Artificial Sequence
  Description of Artificial Sequence Synthetic polynucleotide
catcaagccctt

SEQ ID NO: 508 - DNA, 12 bp, Artificial Sequence
  Description of Artificial Sequence Synthetic polynucleotide
tttaccgagcaa

SEQ ID NO: 509 - DNA, 12 bp, Artificial Sequence
  Description of Artificial Sequence Synthetic polynucleotide
ctttcctgcgcg

SEQ ID NO: 510 - DNA, 12 bp, Artificial Sequence
  Description of Artificial Sequence Synthetic polynucleotide
cttgtgctatct

SEQ ID NO: 511 - DNA, 12 bp, Artificial Sequence
  Description of Artificial Sequence Synthetic polynucleotide
aggaatcgcaaa

SEQ ID NO: 512 - DNA, 12 bp, Artificial Sequence
  Description of Artificial Sequence Synthetic polynucleotide
tctagacacagt

SEQ ID NO: 513 - DNA, 12 bp, Artificial Sequence
  Description of Artificial Sequence Synthetic polynucleotide
actctccatccc

SEQ ID NO: 514 - DNA, 12 bp, Artificial Sequence
  Description of Artificial Sequence Synthetic polynucleotide
acgcctaggcgt

SEQ ID NO: 515 - DNA, 12 bp, Artificial Sequence
  Description of Artificial Sequence Synthetic polynucleotide
cagcaacaactg

SEQ ID NO: 516 - DNA, 12 bp, Artificial Sequence
  Description of Artificial Sequence Synthetic polynucleotide
cttacatgaacc

SEQ ID NO: 517 - DNA, 12 bp, Artificial Sequence
  Description of Artificial Sequence Synthetic polynucleotide
tcctttgtcgtg

SEQ ID NO: 518 - DNA, 12 bp, Artificial Sequence
  Description of Artificial Sequence Synthetic polynucleotide
aagatcgtaaac

SEQ ID NO: 519 - DNA, 12 bp, Artificial Sequence
  Description of Artificial Sequence Synthetic polynucleotide
aggaacatacta

SEQ ID NO: 520 - DNA, 12 bp, Artificial Sequence
  Description of Artificial Sequence Synthetic polynucleotide
gatgaatgatac

SEQ ID NO: 521 - DNA, 12 bp, Artificial Sequence
  Description of Artificial Sequence Synthetic polynucleotide
gagaaataattc

SEQ ID NO: 522 - DNA, 12 bp, Artificial Sequence
  Description of Artificial Sequence Synthetic polynucleotide
gccactcgcatg

SEQ ID NO: 523 - DNA, 12 bp, Artificial Sequence
  Description of Artificial Sequence Synthetic polynucleotide
tgtaaatagccc

SEQ ID NO: 524 - DNA, 12 bp, Artificial Sequence
  Description of Artificial Sequence Synthetic polynucleotide
cataattcgcta

SEQ ID NO: 525 - DNA, 12 bp, Artificial Sequence
  Description of Artificial Sequence Synthetic polynucleotide
cattacgcagtt

SEQ ID NO: 526 - DNA, 12 bp, Artificial Sequence
  Description of Artificial Sequence Synthetic polynucleotide
gagcggatgatt

SEQ ID NO: 527 - DNA, 12 bp, Artificial Sequence
  Description of Artificial Sequence Synthetic polynucleotide
gtacatcgccgc

SEQ ID NO: 528 - DNA, 12 bp, Artificial Sequence
  Description of Artificial Sequence Synthetic polynucleotide
aacactcacttg

SEQ ID NO: 529 - DNA, 12 bp, Artificial Sequence
  Description of Artificial Sequence Synthetic polynucleotide
gtaccgggctac

SEQ ID NO: 530 - DNA, 12 bp, Artificial Sequence
  Description of Artificial Sequence Synthetic polynucleotide
tcgacccattaa

SEQ ID NO: 531 - DNA, 12 bp, Artificial Sequence
  Description of Artificial Sequence Synthetic polynucleotide
gcttaactacgc

SEQ ID NO: 532 - DNA, 12 bp, Artificial Sequence
  Description of Artificial Sequence Synthetic polynucleotide
tactgcccaact

SEQ ID NO: 533 - DNA, 12 bp, Artificial Sequence
  Description of Artificial Sequence Synthetic polynucleotide
tgagcttgatca

SEQ ID NO: 534 - DNA, 12 bp, Artificial Sequence
  Description of Artificial Sequence Synthetic polynucleotide
gtcgcaggacat

SEQ ID NO: 535 - DNA, 12 bp, Artificial Sequence
  Description of Artificial Sequence Synthetic polynucleotide
tagctcgggtaa

SEQ ID NO: 536 - DNA, 12 bp, Artificial Sequence
  Description of Artificial Sequence Synthetic polynucleotide
caacagttccac

SEQ ID NO: 537 - DNA, 12 bp, Artificial Sequence
  Description of Artificial Sequence Synthetic polynucleotide
cgtcaccactca

SEQ ID NO: 538 - DNA, 12 bp, Artificial Sequence
  Description of Artificial Sequence Synthetic polynucleotide
gacacacttgat

SEQ ID NO: 539 - DNA, 12 bp, Artificial Sequence
  Description of Artificial Sequence Synthetic polynucleotide
tgagttgcgttc

SEQ ID NO: 540 - DNA, 12 bp, Artificial Sequence
  Description of Artificial Sequence Synthetic polynucleotide
ctaccctagtcg

SEQ ID NO: 541 - DNA, 12 bp, Artificial Sequence
  Description of Artificial Sequence Synthetic polynucleotide
tagatcttgagt

SEQ ID NO: 542 - DNA, 12 bp, Artificial Sequence
  Description of Artificial Sequence Synthetic polynucleotide
tctacctctaac

SEQ ID NO: 543 - DNA, 12 bp, Artificial Sequence
  Description of Artificial Sequence Synthetic polynucleotide
agacgaaacgga

SEQ ID NO: 544 - DNA, 12 bp, Artificial Sequence
  Description of Artificial Sequence Synthetic polynucleotide
caagggcttccc

SEQ ID NO: 545 - DNA, 12 bp, Artificial Sequence
  Description of Artificial Sequence Synthetic polynucleotide
acgcaattgtac

SEQ ID NO: 546 - DNA, 12 bp, Artificial Sequence
  Description of Artificial Sequence Synthetic polynucleotide
aaataaactcgc

SEQ ID NO: 547 - DNA, 12 bp, Artificial Sequence
  Description of Artificial Sequence Synthetic polynucleotide
accattagccgt

SEQ ID NO: 548 - DNA, 12 bp, Artificial Sequence
  Description of Artificial Sequence Synthetic polynucleotide
acctatccagtg

SEQ ID NO: 549 - DNA, 12 bp, Artificial Sequence
  Description of Artificial Sequence Synthetic polynucleotide
cgtgaatggtga

SEQ ID NO: 550 - DNA, 12 bp, Artificial Sequence
  Description of Artificial Sequence Synthetic polynucleotide
tgctacaaggga

SEQ ID NO: 551 - DNA, 12 bp, Artificial Sequence
  Description of Artificial Sequence Synthetic polynucleotide
cttcagtaccgc

SEQ ID NO: 552 - DNA, 12 bp, Artificial Sequence
  Description of Artificial Sequence Synthetic polynucleotide
caaaccgctcta

SEQ ID NO: 553 - DNA, 12 bp, Artificial Sequence
  Description of Artificial Sequence Synthetic polynucleotide
atgccggtccgc

SEQ ID NO: 554 - DNA, 12 bp, Artificial Sequence
  Description of Artificial Sequence Synthetic polynucleotide
gctgtcgtggga

SEQ ID NO: 555 - DNA, 12 bp, Artificial Sequence
  Description of Artificial Sequence Synthetic polynucleotide
tcccgcctccga

SEQ ID NO: 556 - DNA, 12 bp, Artificial Sequence
  Description of Artificial Sequence Synthetic polynucleotide
gaacctaatttg

SEQ ID NO: 557 - DNA, 12 bp, Artificial Sequence
  Description of Artificial Sequence Synthetic polynucleotide
tccagtatcgac

SEQ ID NO: 558 - DNA, 12 bp, Artificial Sequence
  Description of Artificial Sequence Synthetic polynucleotide
tcacggtcagca

SEQ ID NO: 559 - DNA, 12 bp, Artificial Sequence
  Description of Artificial Sequence Synthetic polynucleotide
tgttgctctctg

SEQ ID NO: 560 - DNA, 12 bp, Artificial Sequence
  Description of Artificial Sequence Synthetic polynucleotide
gtttgcgcaaat

SEQ ID NO: 561 - DNA, 12 bp, Artificial Sequence
  Description of Artificial Sequence Synthetic polynucleotide
caaagcaccgtg

SEQ ID NO: 562 - DNA, 12 bp, Artificial Sequence
  Description of Artificial Sequence Synthetic polynucleotide
ctgaggcatggc

SEQ ID NO: 563 - DNA, 12 bp, Artificial Sequence
  Description of Artificial Sequence Synthetic polynucleotide
caaggatctctt

SEQ ID NO: 564 - DNA, 12 bp, Artificial Sequence
  Description of Artificial Sequence Synthetic polynucleotide
tcaaggcctgtt

SEQ ID NO: 565 - DNA, 12 bp, Artificial Sequence
  Description of Artificial Sequence Synthetic polynucleotide
aacctgtccgta

SEQ ID NO: 566 - DNA, 12 bp, Artificial Sequence
  Description of Artificial Sequence Synthetic polynucleotide
taaccacgaagt

SEQ ID NO: 567 - DNA, 12 bp, Artificial Sequence
  Description of Artificial Sequence Synthetic polynucleotide
ttctcctaggaa

SEQ ID NO: 568 - DNA, 12 bp, Artificial Sequence
  Description of Artificial Sequence Synthetic polynucleotide
aaattgtacttc

SEQ ID NO: 569 - DNA, 12 bp, Artificial Sequence
  Description of Artificial Sequence Synthetic polynucleotide
ctccaccgagtt

SEQ ID NO: 570 - DNA, 12 bp, Artificial Sequence
  Description of Artificial Sequence Synthetic polynucleotide
cagttgtattcg

SEQ ID NO: 571 - DNA, 12 bp, Artificial Sequence
  Description of Artificial Sequence Synthetic polynucleotide
ttgggtagcagc

SEQ ID NO: 572 - DNA, 12 bp, Artificial Sequence
  Description of Artificial Sequence Synthetic polynucleotide
tatatcgcttgc

SEQ ID NO: 573 - DNA, 12 bp, Artificial Sequence
  Description of Artificial Sequence Synthetic polynucleotide
gactcgttgacc

SEQ ID NO: 574 - DNA, 12 bp, Artificial Sequence
  Description of Artificial Sequence Synthetic polynucleotide
taactctggccc

SEQ ID NO: 575 - DNA, 12 bp, Artificial Sequence
  Description of Artificial Sequence Synthetic polynucleotide
gcactaggtttg

SEQ ID NO: 576 - DNA, 12 bp, Artificial Sequence
  Description of Artificial Sequence Synthetic polynucleotide
catgcttccgtt

SEQ ID NO: 577 - DNA, 12 bp, Artificial Sequence
  Description of Artificial Sequence Synthetic polynucleotide
cacctgtgatga

SEQ ID NO: 578 - DNA, 12 bp, Artificial Sequence
  Description of Artificial Sequence Synthetic polynucleotide
acctatttaaga

SEQ ID NO: 579 - DNA, 12 bp, Artificial Sequence
  Description of Artificial Sequence Synthetic polynucleotide
gcacgtaggtaa

SEQ ID NO: 580 - DNA, 12 bp, Artificial Sequence
  Description of Artificial Sequence Synthetic polynucleotide
cagtactgttcc

SEQ ID NO: 581 - DNA, 12 bp, Artificial Sequence
  Description of Artificial Sequence Synthetic polynucleotide
gttaggaaccta

SEQ ID NO: 582 - DNA, 12 bp, Artificial Sequence
  Description of Artificial Sequence Synthetic polynucleotide
tagattgctgat

SEQ ID NO: 583 - DNA, 12 bp, Artificial Sequence
  Description of Artificial Sequence Synthetic polynucleotide
atgagtcatagt

SEQ ID NO: 584 - DNA, 12 bp, Artificial Sequence
  Description of Artificial Sequence Synthetic polynucleotide
agaaatgatccg

SEQ ID NO: 585 - DNA, 12 bp, Artificial Sequence
  Description of Artificial Sequence Synthetic polynucleotide
gatattcatccg

SEQ ID NO: 586 - DNA, 12 bp, Artificial Sequence
  Description of Artificial Sequence Synthetic polynucleotide
tcggttccgtcc

SEQ ID NO: 587 - DNA, 12 bp, Artificial Sequence
  Description of Artificial Sequence Synthetic polynucleotide
agacacaggtag

SEQ ID NO: 588 - DNA, 12 bp, Artificial Sequence
  Description of Artificial Sequence Synthetic polynucleotide
aggttgggccgc

SEQ ID NO: 589 - DNA, 12 bp, Artificial Sequence
  Description of Artificial Sequence Synthetic polynucleotide
gtagaacctctg

SEQ ID NO: 590 - DNA, 12 bp, Artificial Sequence
  Description of Artificial Sequence Synthetic polynucleotide
aactatgcgaaa

SEQ ID NO: 591 - DNA, 12 bp, Artificial Sequence
  Description of Artificial Sequence Synthetic polynucleotide
tgcgacgccacg

SEQ ID NO: 592 - DNA, 12 bp, Artificial Sequence
  Description of Artificial Sequence Synthetic polynucleotide
cgtaagtcgcgt

SEQ ID NO: 593 - DNA, 12 bp, Artificial Sequence
  Description of Artificial Sequence Synthetic polynucleotide
ctgagacttgtt

SEQ ID NO: 594 - DNA, 12 bp, Artificial Sequence
  Description of Artificial Sequence Synthetic polynucleotide
agtcagtaagag

SEQ ID NO: 595 - DNA, 12 bp, Artificial Sequence
  Description of Artificial Sequence Synthetic polynucleotide
cagcgaagcgaa

SEQ ID NO: 596 - DNA, 12 bp, Artificial Sequence
  Description of Artificial Sequence Synthetic polynucleotide
aatgttgtgcga

SEQ ID NO: 597 - DNA, 12 bp, Artificial Sequence
  Description of Artificial Sequence Synthetic polynucleotide
ttccatatcacc

SEQ ID NO: 598 - DNA, 12 bp, Artificial Sequence
  Description of Artificial Sequence Synthetic polynucleotide
gtctcaaattgt

SEQ ID NO: 599 - DNA, 12 bp, Artificial Sequence
  Description of Artificial Sequence Synthetic polynucleotide
gcgggatgttta

SEQ ID NO: 600 - DNA, 12 bp, Artificial Sequence
  Description of Artificial Sequence Synthetic polynucleotide
gatactaaggac

SEQ ID NO: 601 - DNA, 12 bp, Artificial Sequence
  Description of Artificial Sequence Synthetic polynucleotide
cgaaacattaat

SEQ ID NO: 602 - DNA, 12 bp, Artificial Sequence
  Description of Artificial Sequence Synthetic polynucleotide
tacgataggtca

SEQ ID NO: 603 - DNA, 12 bp, Artificial Sequence
  Description of Artificial Sequence Synthetic polynucleotide
gcacaatgcacg

SEQ ID NO: 604 - DNA, 12 bp, Artificial Sequence
  Description of Artificial Sequence Synthetic polynucleotide
gtccaccgtcaa

SEQ ID NO: 605 - DNA, 12 bp, Artificial Sequence
  Description of Artificial Sequence Synthetic polynucleotide
tgaatccaacga

SEQ ID NO: 606 - DNA, 12 bp, Artificial Sequence
  Description of Artificial Sequence Synthetic polynucleotide
gacaatcgtcag

SEQ ID NO: 607 - DNA, 12 bp, Artificial Sequence
  Description of Artificial Sequence Synthetic polynucleotide
tcgcttgatatt

SEQ ID NO: 608 - DNA, 12 bp, Artificial Sequence
  Description of Artificial Sequence Synthetic polynucleotide
acacacaccgct

SEQ ID NO: 609 - DNA, 12 bp, Artificial Sequence
  Description of Artificial Sequence Synthetic polynucleotide
tgaccaccaata

SEQ ID NO: 610 - DNA, 12 bp, Artificial Sequence
  Description of Artificial Sequence Synthetic polynucleotide
cgcctgggtaag

SEQ ID NO: 611 - DNA, 12 bp, Artificial Sequence
  Description of Artificial Sequence Synthetic polynucleotide
tcggtatcagtg

SEQ ID NO: 612 - DNA, 12 bp, Artificial Sequence
  Description of Artificial Sequence Synthetic polynucleotide
gagtcctgatat

SEQ ID NO: 613 - DNA, 12 bp, Artificial Sequence
  Description of Artificial Sequence Synthetic polynucleotide
cactacacatgt

SEQ ID NO: 614 - DNA, 12 bp, Artificial Sequence
  Description of Artificial Sequence Synthetic polynucleotide
cagtaattccat

SEQ ID NO: 615 - DNA, 12 bp, Artificial Sequence
  Description of Artificial Sequence Synthetic polynucleotide
aattaaacaccg

SEQ ID NO: 616 - DNA, 12 bp, Artificial Sequence
  Description of Artificial Sequence Synthetic polynucleotide
gcagggcttgca

SEQ ID NO: 617 - DNA, 12 bp, Artificial Sequence
  Description of Artificial Sequence Synthetic polynucleotide
acccaagccatt

SEQ ID NO: 618 - DNA, 12 bp, Artificial Sequence
  Description of Artificial Sequence Synthetic polynucleotide
aattatactgtg

SEQ ID NO: 619 - DNA, 12 bp, Artificial Sequence
  Description of Artificial Sequence Synthetic polynucleotide
gagcagcgaagt

SEQ ID NO: 620 - DNA, 12 bp, Artificial Sequence
  Description of Artificial Sequence Synthetic polynucleotide
cgccgtaccgta

SEQ ID NO: 621 - DNA, 12 bp, Artificial Sequence
  Description of Artificial Sequence Synthetic polynucleotide
ttagctccggtt

SEQ ID NO: 622 - DNA, 12 bp, Artificial Sequence
  Description of Artificial Sequence Synthetic polynucleotide
gatacgtagatc

SEQ ID NO: 623 - DNA, 12 bp, Artificial Sequence
  Description of Artificial Sequence Synthetic polynucleotide
acttcactccta

SEQ ID NO: 624 - DNA, 12 bp, Artificial Sequence
  Description of Artificial Sequence Synthetic polynucleotide
aggttcattaga

SEQ ID NO: 625 - DNA, 12 bp, Artificial Sequence
  Description of Artificial Sequence Synthetic polynucleotide
tggtcatataat

SEQ ID NO: 626 - DNA, 12 bp, Artificial Sequence
  Description of Artificial Sequence Synthetic polynucleotide
gaaaggcgtact

SEQ ID NO: 627 - DNA, 12 bp, Artificial Sequence
  Description of Artificial Sequence Synthetic polynucleotide
aacgcctggaag

SEQ ID NO: 628 - DNA, 12 bp, Artificial Sequence
  Description of Artificial Sequence Synthetic polynucleotide
aaggtagtggca

SEQ ID NO: 629 - DNA, 12 bp, Artificial Sequence
  Description of Artificial Sequence Synthetic polynucleotide
tggccaatccag

SEQ ID NO: 630 - DNA, 12 bp, Artificial Sequence
  Description of Artificial Sequence Synthetic polynucleotide
tcgacagcgatt

SEQ ID NO: 631 - DNA, 12 bp, Artificial Sequence
  Description of Artificial Sequence Synthetic polynucleotide
ttaaccaccgtt

SEQ ID NO: 632 - DNA, 12 bp, Artificial Sequence
  Description of Artificial Sequence Synthetic polynucleotide
tagagttctatc

SEQ ID NO: 633 - DNA, 12 bp, Artificial Sequence
  Description of Artificial Sequence Synthetic polynucleotide
accagattggag

SEQ ID NO: 634 - DNA, 12 bp, Artificial Sequence
  Description of Artificial Sequence Synthetic polynucleotide
atccttacttta

SEQ ID NO: 635 - DNA, 12 bp, Artificial Sequence
  Description of Artificial Sequence Synthetic polynucleotide
gctacgcggtgc

SEQ ID NO: 636 - DNA, 12 bp, Artificial Sequence
  Description of Artificial Sequence Synthetic polynucleotide
tacttctttgaa

SEQ ID NO: 637 - DNA, 12 bp, Artificial Sequence
  Description of Artificial Sequence Synthetic polynucleotide
acaagagatgct

SEQ ID NO: 638 - DNA, 12 bp, Artificial Sequence
  Description of Artificial Sequence Synthetic polynucleotide
acccaggatgcc

SEQ ID NO: 639 - DNA, 12 bp, Artificial Sequence
  Description of Artificial Sequence Synthetic polynucleotide
gtagttggaaca

SEQ ID NO: 640 - DNA, 12 bp, Artificial Sequence
  Description of Artificial Sequence Synthetic polynucleotide
tctacatttctg

SEQ ID NO: 641 - DNA, 12 bp, Artificial Sequence
  Description of Artificial Sequence Synthetic polynucleotide
gtgcgccgcttc

SEQ ID NO: 642 - DNA, 12 bp, Artificial Sequence
  Description of Artificial Sequence Synthetic polynucleotide
gaacatcagaat

SEQ ID NO: 643 - DNA, 12 bp, Artificial Sequence
  Description of Artificial Sequence Synthetic polynucleotide
gccgaagctgtc

SEQ ID NO: 644 - DNA, 12 bp, Artificial Sequence
  Description of Artificial Sequence Synthetic polynucleotide
aaggacccgaat

SEQ ID NO: 645 - DNA, 12 bp, Artificial Sequence
  Description of Artificial Sequence Synthetic polynucleotide
ctttgccgttat

SEQ ID NO: 646 - DNA, 12 bp, Artificial Sequence
  Description of Artificial Sequence Synthetic polynucleotide
aatctctgccat

SEQ ID NO: 647 - DNA, 12 bp, Artificial Sequence
  Description of Artificial Sequence Synthetic polynucleotide
cgagattaccag

SEQ ID NO: 648 - DNA, 12 bp, Artificial Sequence
  Description of Artificial Sequence Synthetic polynucleotide
gctcgcagctac

SEQ ID NO: 649 - DNA, 12 bp, Artificial Sequence
  Description of Artificial Sequence Synthetic polynucleotide
actcagcccgtt

SEQ ID NO: 650 - DNA, 12 bp, Artificial Sequence
  Description of Artificial Sequence Synthetic polynucleotide
tgatcgcatgca

SEQ ID NO: 651 - DNA, 12 bp, Artificial Sequence
  Description of Artificial Sequence Synthetic polynucleotide
ttcaacagatac

SEQ ID NO: 652 - DNA, 12 bp, Artificial Sequence
  Description of Artificial Sequence Synthetic polynucleotide
gctgatagaagt

SEQ ID NO: 653 - DNA, 12 bp, Artificial Sequence
  Description of Artificial Sequence Synthetic polynucleotide
gtagaatagaat

SEQ ID NO: 654 - DNA, 12 bp, Artificial Sequence
  Description of Artificial Sequence Synthetic polynucleotide
ctcgagcgataa

SEQ ID NO: 655 - DNA, 12 bp, Artificial Sequence
  Description of Artificial Sequence Synthetic polynucleotide
tggtagcggcca

SEQ ID NO: 656 - DNA, 12 bp, Artificial Sequence
  Description of Artificial Sequence Synthetic polynucleotide
aaacggtcttga

SEQ ID NO: 657 - DNA, 12 bp, Artificial Sequence
  Description of Artificial Sequence Synthetic polynucleotide
cagcgcgtccct

SEQ ID NO: 658 - DNA, 12 bp, Artificial Sequence
  Description of Artificial Sequence Synthetic polynucleotide
tctccgatataa

SEQ ID NO: 659 - DNA, 12 bp, Artificial Sequence
  Description of Artificial Sequence Synthetic polynucleotide
gacatcgccttt

SEQ ID NO: 660 - DNA, 12 bp, Artificial Sequence
  Description of Artificial Sequence Synthetic polynucleotide
tacctagattcg

SEQ ID NO: 661 - DNA, 12 bp, Artificial Sequence
  Description of Artificial Sequence Synthetic polynucleotide
tgcaagctctcg

SEQ ID NO: 662 - DNA, 12 bp, Artificial Sequence
  Description of Artificial Sequence Synthetic polynucleotide
cattagccatag

SEQ ID NO: 663 - DNA, 12 bp, Artificial Sequence
  Description of Artificial Sequence Synthetic polynucleotide
tgtcctcaagca

SEQ ID NO: 664 - DNA, 12 bp, Artificial Sequence
  Description of Artificial Sequence Synthetic polynucleotide
tggctgcaaata

SEQ ID NO: 665 - DNA, 12 bp, Artificial Sequence
  Description of Artificial Sequence Synthetic polynucleotide
aacctcatgcag

SEQ ID NO: 666 - DNA, 12 bp, Artificial Sequence
  Description of Artificial Sequence Synthetic polynucleotide
agagctcgaacg

SEQ ID NO: 667 - DNA, 12 bp, Artificial Sequence
  Description of Artificial Sequence Synthetic polynucleotide
attcttcgtgct

SEQ ID NO: 668 - DNA, 12 bp, Artificial Sequence
  Description of Artificial Sequence Synthetic polynucleotide
gaaccaagccta

SEQ ID NO: 669 - DNA, 12 bp, Artificial Sequence
  Description of Artificial Sequence Synthetic polynucleotide
agttactacaag

SEQ ID NO: 670 - DNA, 12 bp, Artificial Sequence
  Description of Artificial Sequence Synthetic polynucleotide
ttgatcccgaat

SEQ ID NO: 671 - DNA, 12 bp, Artificial Sequence
  Description of Artificial Sequence Synthetic polynucleotide
cttggcaccagt

SEQ ID NO: 672 - DNA, 12 bp, Artificial Sequence
  Description of Artificial Sequence Synthetic polynucleotide
gaagaggtaacc

SEQ ID NO: 673 - DNA, 12 bp, Artificial Sequence
  Description of Artificial Sequence Synthetic polynucleotide
acgaccgggttg

SEQ ID NO: 674 - DNA, 12 bp, Artificial Sequence
  Description of Artificial Sequence Synthetic polynucleotide
gcatttagctca

SEQ ID NO: 675 - DNA, 12 bp, Artificial Sequence
  Description of Artificial Sequence Synthetic polynucleotide
tgtcgttgccga

SEQ ID NO: 676 - DNA, 12 bp, Artificial Sequence
  Description of Artificial Sequence Synthetic polynucleotide
agaaagcttctg

SEQ ID NO: 677 - DNA, 12 bp, Artificial Sequence
  Description of Artificial Sequence Synthetic polynucleotide
tagtggtctgca

SEQ ID NO: 678 - DNA, 12 bp, Artificial Sequence
  Description of Artificial Sequence Synthetic polynucleotide
gtcgaggtctac

SEQ ID NO: 679 - DNA, 12 bp, Artificial Sequence
  Description of Artificial Sequence Synthetic polynucleotide
tcttcagcgacc

SEQ ID NO: 680 - DNA, 12 bp, Artificial Sequence
  Description of Artificial Sequence Synthetic polynucleotide
tcagctaactca

SEQ ID NO: 681 - DNA, 12 bp, Artificial Sequence
  Description of Artificial Sequence Synthetic polynucleotide
tccaatcgacct

SEQ ID NO: 682 - DNA, 12 bp, Artificial Sequence
  Description of Artificial Sequence Synthetic polynucleotide
cggtctgatata

SEQ ID NO: 683 - DNA, 12 bp, Artificial Sequence
  Description of Artificial Sequence Synthetic polynucleotide
gagggcaattca

SEQ ID NO: 684 - DNA, 12 bp, Artificial Sequence
  Description of Artificial Sequence Synthetic polynucleotide
acggtcttcgag

SEQ ID NO: 685 - DNA, 12 bp, Artificial Sequence
  Description of Artificial Sequence Synthetic polynucleotide
accgagttgtct

SEQ ID NO: 686 - DNA, 12 bp, Artificial Sequence
  Description of Artificial Sequence Synthetic polynucleotide
tgcgagcgtccc

SEQ ID NO: 687 - DNA, 12 bp, Artificial Sequence
  Description of Artificial Sequence Synthetic polynucleotide
cttgcacatact

SEQ ID NO: 688 - DNA, 12 bp, Artificial Sequence
  Description of Artificial Sequence Synthetic polynucleotide
accttagtgtcc

SEQ ID NO: 689 - DNA, 12 bp, Artificial Sequence
  Description of Artificial Sequence Synthetic polynucleotide
ctgtagttctcg

SEQ ID NO: 690 - DNA, 12 bp, Artificial Sequence
  Description of Artificial Sequence Synthetic polynucleotide
aggcccttagtc

SEQ ID NO: 691 - DNA, 12 bp, Artificial Sequence
  Description of Artificial Sequence Synthetic polynucleotide
caacgatactaa

SEQ ID NO: 692 - DNA, 12 bp, Artificial Sequence
  Description of Artificial Sequence Synthetic polynucleotide
gccaaatgcgaa

SEQ ID NO: 693 - DNA, 12 bp, Artificial Sequence
  Description of Artificial Sequence Synthetic polynucleotide
tccaaagttctc

SEQ ID NO: 694 - DNA, 12 bp, Artificial Sequence
  Description of Artificial Sequence Synthetic polynucleotide
acccggactatc

SEQ ID NO: 695 - DNA, 12 bp, Artificial Sequence
  Description of Artificial Sequence Synthetic polynucleotide
caacaatgtgaa

SEQ ID NO: 696 - DNA, 12 bp, Artificial Sequence
  Description of Artificial Sequence Synthetic polynucleotide
gtcatcattaca

SEQ ID NO: 697 - DNA, 12 bp, Artificial Sequence
  Description of Artificial Sequence Synthetic polynucleotide
cgtatttgaact

SEQ ID NO: 698 - DNA, 12 bp, Artificial Sequence
  Description of Artificial Sequence Synthetic polynucleotide
cacacatgtgct

SEQ ID NO: 699 - DNA, 12 bp, Artificial Sequence
  Description of Artificial Sequence Synthetic polynucleotide
aggtctcgatag

SEQ ID NO: 700 - DNA, 12 bp, Artificial Sequence
  Description of Artificial Sequence Synthetic polynucleotide
cgaaattgaaac

SEQ ID NO: 701 - DNA, 12 bp, Artificial Sequence
  Description of Artificial Sequence Synthetic polynucleotide
tctggcaaacaa

SEQ ID NO: 702 - DNA, 12 bp, Artificial Sequence
  Description of Artificial Sequence Synthetic polynucleotide
cggattgaacga

SEQ ID NO: 703 - DNA, 12 bp, Artificial Sequence
  Description of Artificial Sequence Synthetic polynucleotide
tatcaagtgatc

SEQ ID NO: 704 - DNA, 12 bp, Artificial Sequence
  Description of Artificial Sequence Synthetic polynucleotide
acagtaagtgta

SEQ ID NO: 705 - DNA, 12 bp, Artificial Sequence
  Description of Artificial Sequence Synthetic polynucleotide
atgcgcgcagca

SEQ ID NO: 706 - DNA, 12 bp, Artificial Sequence
  Description of Artificial Sequence Synthetic polynucleotide
agcggacgtgca

SEQ ID NO: 707 - DNA, 12 bp, Artificial Sequence
  Description of Artificial Sequence Synthetic polynucleotide
taacgtatgaac

SEQ ID NO: 708 - DNA, 12 bp, Artificial Sequence
  Description of Artificial Sequence Synthetic polynucleotide
attgccctcatg

SEQ ID NO: 709 - DNA, 12 bp, Artificial Sequence
  Description of Artificial Sequence Synthetic polynucleotide
cttccaacataa

SEQ ID NO: 710 - DNA, 12 bp, Artificial Sequence
  Description of Artificial Sequence Synthetic polynucleotide
cgcccttaccca

SEQ ID NO: 711 - DNA, 12 bp, Artificial Sequence
  Description of Artificial Sequence Synthetic polynucleotide
caacaatcgatt

SEQ ID NO: 712 - DNA, 12 bp, Artificial Sequence
  Description of Artificial Sequence Synthetic polynucleotide
caggttcgccgt

SEQ ID NO: 713 - DNA, 12 bp, Artificial Sequence
  Description of Artificial Sequence Synthetic polynucleotide
tgatgcaatagt

SEQ ID NO: 714 - DNA, 12 bp, Artificial Sequence
  Description of Artificial Sequence Synthetic polynucleotide
aatccgctccgt

SEQ ID NO: 715 - DNA, 12 bp, Artificial Sequence
  Description of Artificial Sequence Synthetic polynucleotide
ctcggcgttcat

SEQ ID NO: 716 - DNA, 12 bp, Artificial Sequence
  Description of Artificial Sequence Synthetic polynucleotide
tgttcaggtccc

SEQ ID NO: 717 - DNA, 12 bp, Artificial Sequence
  Description of Artificial Sequence Synthetic polynucleotide
catccttaagtc

SEQ ID NO: 718 - DNA, 12 bp, Artificial Sequence
  Description of Artificial Sequence Synthetic polynucleotide
aggactgttgct

SEQ ID NO: 719 - DNA, 12 bp, Artificial Sequence
  Description of Artificial Sequence Synthetic polynucleotide
ttgcgaactcta

SEQ ID NO: 720 - DNA, 12 bp, Artificial Sequence
  Description of Artificial Sequence Synthetic polynucleotide
gttctggattta

SEQ ID NO: 721 - DNA, 12 bp, Artificial Sequence
  Description of Artificial Sequence Synthetic polynucleotide
ttctggtccacc

SEQ ID NO: 722 - DNA, 12 bp, Artificial Sequence
  Description of Artificial Sequence Synthetic polynucleotide
cacaccaaatca

SEQ ID NO: 723 - DNA, 12 bp, Artificial Sequence
  Description of Artificial Sequence Synthetic polynucleotide
agcgaactgggc

SEQ ID NO: 724 - DNA, 12 bp, Artificial Sequence
  Description of Artificial Sequence Synthetic polynucleotide
gagctcgggctt

SEQ ID NO: 725 - DNA, 12 bp, Artificial Sequence
  Description of Artificial Sequence Synthetic polynucleotide
gctcctgtcggt

SEQ ID NO: 726 - DNA, 12 bp, Artificial Sequence
  Description of Artificial Sequence Synthetic polynucleotide
tgcttccctcaa

SEQ ID NO: 727 - DNA, 12 bp, Artificial Sequence
  Description of Artificial Sequence Synthetic polynucleotide
tggagataattg

SEQ ID NO: 728 - DNA, 12 bp, Artificial Sequence
  Description of Artificial Sequence Synthetic polynucleotide
aatattcaggct

SEQ ID NO: 729 - DNA, 12 bp, Artificial Sequence
  Description of Artificial Sequence Synthetic polynucleotide
tgtatgaatgcc

SEQ ID NO: 730 - DNA, 12 bp, Artificial Sequence
  Description of Artificial Sequence Synthetic polynucleotide
attgagacccac

SEQ ID NO: 731 - DNA, 12 bp, Artificial Sequence
  Description of Artificial Sequence Synthetic polynucleotide
caggatcctatc

SEQ ID NO: 732 - DNA, 12 bp, Artificial Sequence
  Description of Artificial Sequence Synthetic polynucleotide
gtggctcgttct

SEQ ID NO: 733 - DNA, 12 bp, Artificial Sequence
  Description of Artificial Sequence Synthetic polynucleotide
cgtttcgaggcg

SEQ ID NO: 734 - DNA, 48 bp, Artificial Sequence
  Description of Artificial Sequence Synthetic polynucleotide
cggaattccgtctccggaattccgtaataagctgcagcaccagcaagg

SEQ ID NO: 735 - DNA, 24 bp, Artificial Sequence
  Description of Artificial Sequence Synthetic polynucleotide
ccttgctggtgctgcagcttatta

SEQ ID NO: 736 - DNA, 48 bp, Artificial Sequence
  Description of Artificial Sequence Synthetic polynucleotide
gcaagcaaccgcagggttgcttgctagactgtgcacttgaccctaagg

SEQ ID NO: 737 - DNA, 60 bp, Artificial Sequence
  Description of Artificial Sequence Synthetic polynucleotide
cggaattccgtctccggaattccgtaataagctgcagcaccagcagatcggaagagcgtc

SEQ ID NO: 738 - DNA, 36 bp, Artificial Sequence
  Description of Artificial Sequence Synthetic polynucleotide
gacgctcttccgatctgctggtgctgcagcttatta

SEQ ID NO: 739 - DNA, 60 bp, Artificial Sequence
  Description of Artificial Sequence Synthetic polynucleotide
cggaattccgtctccggaattccgtaataagctgcagcaccagctagatcggaagagcgt

SEQ ID NO: 740 - DNA, 60 bp, Artificial Sequence
  Description of Artificial Sequence Synthetic polynucleotide
cggaattccgtctccggaattccgtaataagctgcagcaccagctgagatcggaagagcg

SEQ ID NO: 741 - DNA, 60 bp, Artificial Sequence
  Description of Artificial Sequence Synthetic polynucleotide
cggaattccgtctccggaattccgtaataagctgcagcaccagctaaagatcggaagagc

SEQ ID NO: 742 - DNA, 60 bp, Artificial Sequence
  Description of Artificial Sequence Synthetic polynucleotide
gcaagcaaccgcagggttgcttgctagactgtgcacttgaccctagatcggaagagcaca

SEQ ID NO: 743 - DNA, 20 bp, Artificial Sequence
  Description of Artificial Sequence Synthetic polynucleotide
gctggtgctgcagcttatta

SEQ ID NO: 744 - DNA, 20 bp, Artificial Sequence
  Description of Artificial Sequence Synthetic polynucleotide
agggtcaagtgcacagtcta

(19) United States
(12) Patent Application Publication
(10) Pub. No.: US 2024/0093287 A1
(43) Pub. Date: Mar. 21, 2024

(54) METHODS AND COMPOSITIONS FOR REDUCING INDEX HOPPING

(71) Applicant: Ginkgo Bioworks, Inc., Boston, MA (US)

(72) Inventors: Keith Robison, Andover, MA (US); Douglas G. Smith, Walnut Creek, CA (US); Adam J. Meyer, Boston, MA (US); Andrew J. Mitchell, Framingham, MA (US); Alex Plocik, Auburndale, MA (US); Thomas F. Knight, Belmont, MA (US)

(21) Appl. No.: 18/018,401

(22) PCT Filed: Jul. 30, 2021
(86) PCT No.: PCT/US2021/043994

§ 371 Date: Jan. 27, 2023

Related U.S. Application Data
(60) Provisional application No. 63/059,117, filed on Jul. 30, 2020.
(60) Provisional application No. 63/094,308, filed on Oct. 20, 2020.
(60) Provisional application No. 63/094,301, filed on Oct. 20, 2020.

(51) Int. Cl. C12Q 1/6869

(52) U.S. Cl. CPC C12Q 1/6869

(57) ABSTRACT

The present disclosure relates to compositions and methods for reducing the concentration of extendable free and buried primers relative to amplification product in a sample. The disclosed methods and compositions can be used to reduce or eliminate index hopping in a next generation sequencing (NGS) platform.

### REFERENCE TO A SEQUENCE LISTING

This application contains a Sequence Listing in computer readable form. The computer readable form is incorporated herein by reference. Said ASCII copy, created on Jun. 30, 2023, is named GBB-00301_SL.txt and is 177,494 bytes in size.

### CROSS-REFERENCE TO RELATED APPLICATION

This application is the § 371 National Stage of International Patent Application No.: PCT/US2021/043994, filed Jul. 30, 2021, which claims the benefit of the following U.S. Provisional Application Ser. No. 63/094,301 filed Oct. 20, 2020; 63/094,308 filed Oct. 20, 2020; and 63/059,117 filed Jul. 30, 2020, the entire contents of which are incorporated herein by reference.

### BACKGROUND

Next generation sequencing (NGS) platforms allow for massively parallel sequencing and the generation of enormous amounts of sequencing data. Typically, when NGS platforms are used for diagnostic or other clinical applications each sequencing run is performed on multiple combined patient samples in order to increase the efficiency of the sequencing process. This is accomplished by indexing nucleic acids in each patient sample through the attachment of patient-specific polynucleotide barcodes (e.g., during an amplification step) before combining the samples for sequencing. Following sequencing, these barcode sequences are used to associate sequencing reads back to individual patient samples.

One source of artifacts during multiplex NGS sequencing processes is index hopping, which happens when a barcode sequence specific for one patient attaches to and tags a template nucleic acid from a different patient following the combination of patient samples. Index hopping therefore can result in the creation of sequencing templates labeled with an incorrect polynucleotide barcode. Being improperly indexed, the resulting sequencing read may be associated with the wrong patient, potentially resulting in a false-positive or false-negative result.

As multiplexed NGS assays are being increasingly applied to diagnostic applications, there is a great need in the art for effective compositions and methods for reducing index hopping.

### SUMMARY

In certain aspects, the present disclosure relates to compositions and methods that reduce the incidence of index hopping by reducing the concentration of extendable free and buried primers relative to amplification product in an indexed sample (e.g., following an amplification step) prior to performance of a multiplex next generation sequencing (NGS) assay.

In certain aspects, provided herein is a method for generating a sequencing sample comprising indexed sequencing templates (e.g., a sample for multiplexed NGS sequencing comprising indexed sequencing templates amplified from a plurality of patient samples), the method comprising subjecting a sample comprising indexed sequencing templates and extendable free and/or buried primers to a process that reduces the concentration of free or buried primers relative to the concentration of indexed sequencing templates to generate a sequencing sample that is less prone to index hopping when subjected to a next generation sequencing (NGS) assay.

Numerous embodiments are further provided that can be applied to any aspect disclosed herein and/or combined with any other embodiment described.

For example, in some embodiments, the indexed sequencing templates comprise at least one unique index sequence. In some embodiments, the indexed sequencing templates comprise unique dual index (UDI) sequences. In some embodiments, the indexed sequencing templates are indexed amplification products (e.g. the combined products of a plurality of amplification reactions used to associate barcode sequences with patient nucleic acid sequences). In some embodiments, the indexed sequencing templates comprise at least 50, at least 100, at least 200, at least 250, at least 300, at least 350, at least 400, at least 450, at least 500, at least 550, at least 600, at least 600, at least 650, at least 700, at least 750, at least 800, at least 850, at least 900, at least 950, at least 1000, at least 1250, at least 1500, at least 1750, at least 2000, at least 2250, at least 2500, at least 2750, at least 3000, at least 3250, at least 3500, at least 3750, at least 4000, or more unique barcode sequences and/or unique barcode sequence pairs (e.g., if a UDI system is used). In certain embodiments, the method further comprises performing a next generation sequencing (NGS) assay on the sequencing sample.

In some embodiments, the process that reduces the relative concentration of extendable free or buried primers comprises performing high pressure liquid chromatography (HPLC). In certain embodiments, the HPLC is performed under denaturing conditions.

In some embodiments, the process that reduces the relative concentration of extendable free or buried primers comprises contacting the indexed sequencing template with terminal deoxy transferase (TdT) and dideoxynucleotide triphosphates (ddNTPs). In certain embodiments, the method also comprises contacting the indexed sequencing template with a reagent that frees buried primers. In some embodiments, the reagent that frees buried primers is a protein reagent (e.g., single stranded binding protein (SSB), recA, or UvrB).

In certain embodiments, the process that reduces the relative concentration of extendable free or buried primers comprises contacting the indexed sequencing template with a scavenger nucleic acid molecule, which comprises a sequence complementary to a sequence of the primer. In some embodiments, the scavenger nucleic acid molecule comprises a 3′ ddNTP.

In some embodiments, the process that reduces the relative concentration of free or buried primers comprises contacting the indexed sequencing template with a killer oligonucleotide and a ligase, wherein the killer oligonucleotide comprises a region having a sequence complementary to that of a region of the primer, and wherein when the killer oligonucleotide is hybridized to the primer, the ligase is capable of ligating the killer oligonucleotide to the primer. In some embodiments, the killer oligonucleotide comprises a 5′ phosphate and/or a 3′ ddNTP. In some embodiments, the ligase is TAQ ligase.

In certain embodiments, the process that reduces the relative concentration of extendable free or buried primers comprises (i) performing an amplification reaction on the indexed sequencing template using primers comprising a capture moiety to produce a capture moiety-tagged amplification product, and (ii) purifying the capture moiety-tagged amplification product. In one embodiment, the capture moiety comprises biotin. In certain embodiments, various methods for reducing the relative concentration of extendable free or buried primers can be combined (e.g., performed simultaneously or sequentially).

In certain embodiments, any of the steps in any of these various methods can be assisted by or performed by machines such as computer-controlled robots at individual stations; and the samples can be shuttled between stations. In some embodiments, the shuttling is performed by trucks or cars carrying the samples on the track, and in some embodiments, the shuttling is performed using a magnetic-levitation (maglev) system.

In certain embodiments, in any method, any two or more processes for reducing the relative concentration of extendable free or buried primers can be combined (e.g., performed simultaneously or sequentially).

In certain aspects, provided herein is a sequencing sample generated according to a method described above.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a diagram showing the functional domains of an example primer of the present disclosure.

FIG. 2 is a diagram showing free-primers and “buried primers,” the presence of either of which can lead to index hopping.

FIG. 3 shows histograms that show the number of index pairs for the number of reads for a given forbidden index pair.

FIGS. 4A-4E illustrate the differences of amplification platforms used in the NovaSeq and NextSeq Illumina platforms. FIG. 4A is an illustration showing the expected products of an example of dual indexing approach provided herein. FIG. 4B is an illustration showing an example of how free or buried primers can lead to index hopping and false positives (e.g., in the NovaSeq platform). FIG. 4C is an illustration showing that payload from a sample coded a dual index represented as 123/456 becomes coded with a dual index of 789/456 after index hopping. If 789/456 is assigned to another sample, this error impacts that sample. Moreover, this error reduces the true count of the sample coded 123/456. FIG. 4D is an illustration showing the PCR-based amplification used for generating templates for the NextSeq platform. FIG. 4E is a graph showing the increased risk of false positives in the NovaSeq platform relative to the NextSeq platform due to index hopping

FIG. 5 is a schematic illustration showing an example of an approach to reducing index hopping that uses a scavenger nucleic acid molecules to extend primers to generate an extension product comprising an irrelevant sequence after the anneal region, resulting in extended primers that can no longer extend off normal templates.

FIG. 6 is a schematic illustration showing an example of an approach to reduce index hopping that uses a DNA polymerase to incorporate a ddNTP onto the 3′ end of a buried primer.

FIGS. 7A-7G illustrate the use of oligonucleotide for sequestering and neutralizing free and/or buried primers. FIG. 7A is an illustration showing a killer oligonucleotide mediated capture process for neutralizing free and/or buried primers. FIG. 7B is a diagram of an example killer oligonucleotide for neutralizing free and/or buried forward primers. FIG. 7B discloses SEQ ID NOS 734-735 and 734, respectively, in order of appearance. FIG. 7C is a diagram of an example killer oligonucleotide for neutralizing free and/or buried reverse primers. FIG. 7C discloses SEQ ID NOS 736 and 736, respectively, in order of appearance. The bold sequences in FIGS. 7B and 7C are not homologous to the primers, thereby ensuring the 3′ of the capture oligonucleotide will not extend during exclusion amplification. FIG. 7D is a diagram showing an example killer oligonucleotide for neutralizing free and/or buried forward primers. In some embodiments, the capture oligonucleotide comprises the reverse complement of the spacer and a TruSeq fragment shorter by the length of the spacer. FIG. 7D discloses SEQ ID NOS 737-738 and 737, respectively, in order of appearance. FIG. 7E shows four different examples of designs of killer oligonucleotides for neutralizing free and/or buried forward primers. FIG. 7E discloses SEQ ID NOS 737 and 739-741, respectively, in order of appearance. FIG. 7F is a diagram of an example killer oligonucleotide for neutralizing free and/or buried reverse primers. FIG. 7F discloses SEQ ID NOS 742 and 742, respectively, in order of appearance. FIG. 7G is a diagram showing examples of neutralized forward and reverse primers. FIG. 7G discloses SEQ ID NOS 743, 734, 744 and 736, respectively, in order of appearance.

FIG. 8 is a schematic illustration showing an example of an approach to reduce index hopping by performing an amplification reaction using biotinylated primers to generate a biotinylated amplification product that can then be purified away from free and/or buried primers.

FIG. 9 is a diagram showing an overview of an example data analysis process disclosed.

FIG. 10 shows a set of histograms showing the effect of different examples of protocols for reducing relative concentration of free and/or buried primers provided herein on index hopping.

FIGS. 11A and 11B illustrate HPLC purification of amplicons. FIG. 11A is a chromatogram showing the peaks for primers (left-most peaks) and amplicons (right-most peak). The blue data represents the amplified sample, and the green line represents only primers. FIG. 11B is a chromatogram showing only the data from the amplified sample. Fraction C2 was specifically collected and moved forward for sequencing.

FIG. 12 is a graph showing index hopping observed in No Template Controls (NTCs) amplicons not treated to reduce free or buried primers (DX-071) and amplicons treated with Taq DNA polymerase and ddNTPs (DX-105).

FIGS. 13A-13C illustrate HPLC purification of amplicons using denaturing conditions (pH=12) and ion exchange chromatography columns with a long run-time protocol and the purification's impact on index hopping. FIG. 13A is a chromatogram of the HPLC purification of the amplified sample. FIG. 13B is an enhanced view of the cluster of peaks observed in FIG. 13A. FIG. 13C is a graph showing index hopping observed in No Template Controls (NTCs) amplicons not treated to reduce free or buried primers (DX-071) and amplicons purified using the HPLC long run-time method (DX-094).

FIGS. 14A-14C illustrate HPLC purification of amplicons using denaturing conditions (pH=12) and ion exchange chromatography columns with a short run-time protocol and the purification's impact on index hopping. FIG. 14A is a chromatogram of the HPLC purification of the amplified sample. FIG. 14B is an enhanced view of the major of peak observed in FIG. 14A. FIG. 14C is a graph showing index hopping observed in No Template Controls (NTCs) amplicons not treated to reduce free or buried primers (DX-071) and amplicons purified using the HPLC short run-time method (DX-097).

FIGS. 15A-15C illustrate HPLC purification of using denaturing conditions (85° C.) and ion-pairing reverse phase chromatography and the purification's impact on index hopping. FIG. 15A is a chromatogram of the HPLC purification of the amplified sample. FIG. 15B is an enhanced view of the major of peak observed in FIG. 15A. FIG. 15C is a graph showing index hopping observed in No Template Controls (NTCs) amplicons not treated to reduce free or buried primers (DX-071) and amplicons purified using denaturing conditions (85° C.) and ion-pairing reverse phase chromatography (DX-102).

FIG. 16 is a graph illustrating the differences in index hopping at different primer conditions.

### DETAILED DESCRIPTION

In certain aspects, provided are methods for reducing or eliminating index hopping in next generation sequencing (NGS) platforms, as well as compositions and kits used in the performance of such methods.

The present disclosure pertains to methods and compositions for reducing or eliminating the incidence of index hopping in next generation sequencing (NGS) applications. This disclosure is based, at least in part, on the discovery that performing certain processes that reduce the relative concentration of extendable free and/or buried primers in a sample comprising a indexed sequencing templates (e.g., an indexed amplification products) prior to sequencing reduces index hopping in NGS platforms. For example, this can be accomplished by reducing the total amount of free and/or buried primers and/or by neutralizing present free and/or buried primers such that they cannot be extended during the sequencing process. In certain embodiments, the processes provided herein can be used in combination to further reduce the relative concentration of extendable free and/or buried primers. Thus, in certain aspects, provided herein are methods for reducing the relative concentration of extendible free and/or buried primers that can be applied to an indexed sample prior to the performance of multiplex NGS in order to reduce or eliminate the incidence of index hopping.

Provided herein are various processes for reducing or eliminating index hopping in next generation sequencing (NGS) platforms, each of which can be performed alone or in combination with other index hopping reduction processes. Thus, in some embodiments, any step, reagent, or equipment in any method described can be combined with any other step, protocol, reagent, equipment, etc., of any other method described. In some embodiments, the present disclosure pertains to a method for reducing or eliminating index hopping during a NGS assay, wherein the method comprises any two or more step(s), protocol, reagent(s), equipment, etc., described for any method described.

In certain embodiments of the methods provided herein, pooled indexed samples are treated with a process for reducing index hopping provided herein and then assayed for the presence or absence of a nucleic acid molecule using a NGS assay. In one embodiment, the method of generating the indexed samples comprises performing a multiplex reverse transcription polymerase chain reaction (RT-PCR) with barcoded (e.g., DNA barcoded) primers.

In some embodiments, a process for reducing or eliminating index hopping in next generation sequencing (NGS) platforms provided herein can be used in combination with a method for detecting a nucleic acid molecule in a sample that comprises the steps of: collecting a sample from an individual or a pool of individuals; preparing the sample (e.g., extracting RNA from the sample); amplifying nucleic acids in the sample, using primers which are complementary to at least a portion of a target nucleic acid sequence or a control nucleic acid sequence and which comprise a unique DNA barcode (index); optionally, cleaning up the sample; optionally, combining products of the amplification of multiple samples; sequencing the amplified nucleic acids; deconvoluting the results using the DNA barcodes (indexes) to correlate results with individuals or pools of individuals; and communicating the results to the individuals or pools of individuals.

### Samples

In certain embodiments, the methods provided herein are directed to processing indexed sequencing templates (e.g., indexed amplification products) generated by an amplification or primer extension reaction of a target nucleic acid in a sample. In some embodiments, the sample used to generate the indexed sequencing templates, is a biological sample that contains nucleic acid molecules. Non-limiting examples of the source of the sample include saliva, blood, plasma, serum, lymph fluid, nasal discharge, or aspirate, or a sample obtained for example by surgery or autopsy. In some embodiments, the sample is a saliva, blood, serum, plasma, urine, or a mucous sample, or a test sample derived from a saliva, blood, serum, plasma, urine, or a mucous sample. In some embodiments, the sample is a sample of saliva and/or a sample derived from saliva. In certain embodiments, the sample is a human sample (e.g., a patient sample).

In some embodiments, the sample is a pool sample (or pooled sample) collected from a plurality of individuals (e.g., at least 2, 3, 4, 5, 6, 7, 8, 9, 10, 11, 12, 13, 14, 15, 16, 17, 18, 19, 20, 25, 30, 35, 40, 45, 50, 100, 200, 300, 400, 500, 600, 700, 800, 900, 1000, 1500, 2000, 2500, 3000, 3500, 4000, or more individuals). In some embodiments, pool testing is effective for economically diagnostically testing groups of individuals, as the testing of pool samples consumes fewer reagents, less lab time, etc., than testing the corresponding individual samples. In some embodiments, a pooled sample is collected from a plurality of individuals who have each previously been tested to be negative in a diagnostic test. In some embodiments, once an individual in the pool is tested to be positive in a diagnostic test, the individual is removed from the pool. In some embodiments, if a pooled sample is tested to be positive, samples from each individual are separately tested to determine which individual(s) are positive.

In some embodiments, the sample is a derived from or comprises a cell culture. Methodologies for passaging existing cultures of adherent or suspension mammalian cells are known in the art and can be used to prepare or maintain samples for use in the assays described. Cells can be further propagated, frozen, or used towards other protocols. Such methods for propagating, freezing, or otherwise using cells are known in the art. In some embodiments, the cells are used as controls; for example, HEK293t cells can be used as a control cell that expresses a particular nucleic acid molecule.

In some embodiments, a patient sample is collected and/or prepared using any steps, protocols, reagents, equipment, etc., described and/or known in the art.

### Amplification

In some embodiments, provided herein are methods of preparing a sequencing sample comprising indexed sequencing templates, wherein the indexed sequencing templates are amplification products. In certain embodiments, the methods further comprise the step of generating the indexed sequencing templates from sample nucleic acid molecules. In some embodiments, the nucleic acid molecule is amplified by PCR, including but not limited to RT-PCR. In some embodiments of the methods provided, following sample collection and preparation, and nucleic acid (e.g., DNA or RNA) extraction, the sample (or a portion thereof being tested for comprising a nucleic acid molecule) can be subjected to PCR with various primers to detect the target nucleic acid molecule. Protocols for PCR and RT-PCR are well-known. For example, with RT-PCR, an RNA or control nucleic acids can first be treated with reverse transcriptase and a primer (e.g., a primer with an index sequence provided) to create cDNA prior to detection, quantitation and/or amplification.

By “amplification” is meant any process of producing at least one copy of a nucleic acid, or producing multiple copies of a polynucleotide of interest. An amplification product can be RNA (e.g., viral RNA) or DNA (e.g., cDNA), and may include a complementary strand to the target sequence. DNA amplification products can be produced initially through reverse translation and then optionally from further amplification reactions. The amplification product may include all or a portion of a target sequence, and may optionally be labeled. A variety of amplification methods are suitable for use, including polymerase-based methods and ligation-based methods. Examples of amplification techniques include the polymerase chain reaction method (PCR), isothermal amplification, and the like.

Asymmetric amplification reactions may be used to preferentially amplify one strand representing the target sequence that is used for detection. In some cases, the presence and/or amount of the amplification product itself may be used to determine the expression level of a given target sequence. In other instances, the amplification product may be used to hybridize to an array or other substrate comprising sensor polynucleotides which are used to detect and/or quantitate target sequence expression.

The first cycle of amplification in polymerase-based methods typically forms a primer extension product complementary to the template strand. If the template is single-stranded RNA, a polymerase with reverse transcriptase activity is used in the first amplification to reverse transcribe the RNA to DNA, and additional amplification cycles can be performed to copy the primer extension products. The primers for a PCR must, of course, be designed to hybridize to regions in their corresponding template that can produce an amplifiable segment; thus, each primer must hybridize so that its 3′ nucleotide is paired to a nucleotide in its complementary template strand that is located 3′ from the 3′ nucleotide of the primer used to replicate that complementary template strand in the PCR.

The target polynucleotide can be amplified by contacting one or more strands of the target polynucleotide with a primer and a polymerase having suitable activity to extend the primer and copy the target polynucleotide to produce a full-length complementary polynucleotide or a smaller portion thereof. Any enzyme having a polymerase activity that can copy the target polynucleotide can be used, including DNA polymerases, RNA polymerases, reverse transcriptases, enzymes having more than one type of polymerase or enzyme activity. The enzyme can be thermolabile or thermostable. Mixtures of enzymes can also be used.

Suitable reaction conditions are chosen to permit amplification of the target polynucleotide, including pH, buffer, ionic strength, presence and concentration of one or more salts, presence and concentration of reactants and cofactors such as nucleotides and magnesium and/or other metal ions (e.g., manganese), optional cosolvents, temperature, thermal cycling profile for amplification schemes comprising a polymerase chain reaction, and may depend in part on the polymerase being used as well as the nature of the sample. Cosolvents include formamide (typically at from about 2 to about 10%), glycerol (typically at from about 5 to about 10%), and DMSO (typically at from about 0.9 to about 10%). Techniques may be used in the amplification scheme in order to minimize the production of false positives or artifacts produced during amplification. These include “touchdown” PCR, hot-start techniques, use of nested primers, or designing PCR primers so that they form stem-loop structures in the event of primer-dimer formation and thus are not amplified. Techniques to accelerate PCR can be used, for example, centrifugal PCR, which allows for greater convection within the sample, and/or infrared heating steps for rapid heating and cooling of the sample. One or more cycles of amplification can be performed. An excess of one primer can be used to produce an excess of one primer extension product during PCR; preferably, the primer extension product produced in excess is the amplification product to be detected. A plurality of different primers may be used to amplify different target polynucleotides or different regions of a particular target polynucleotide within the sample.

An amplification reaction can be performed under conditions that allow an optionally labeled sensor polynucleotide to hybridize to the amplification product during at least part of an amplification cycle. When the assay is performed in this manner, real-time detection of this hybridization event can take place by monitoring for light emission or fluorescence during amplification, as known in the art.

In a non-limiting example of RT-PCR: RT-PCR reaction plate prep happens in parallel, which generates the barcodes and RT-PCR master mix in a 384 well plate (or a microwell array with even more wells, e.g., 1 1,000 well microwell array, a 5,000 well microwell array, a 10,000 well microwell array, a 25,000 well microwell array, a 50,000 well microwell array, a 100,000 well microwell array, a 250,000 well microwell array). In some embodiments, rearray compresses the eluate from RNA extraction into the RT-PCR plate. Once combined, it is sealed and centrifuged a second time, and sent to the post-PCR lab space across the elevated conveyor and through an airlock. Thermal cycling currently happens on a 70 thermal cycler bank. After thermal cycling, these plates are pooled based on a compression algorithm.

### Primers

In various embodiments, primers are provided (e.g., for the preparation indexed sequencing templates processed according to methods provided herein).

In some embodiments, pairs of primers target (e.g., comprise sequences complementary to) specific targets, and within each pair of primers, at least one comprises a DNA barcode (i.e., an index sequence). In some embodiments, within a pair of primers, one is an i5 primer and one is an i7 primer. In some embodiments, within a pair of primers, one is a forward primer and one is a reverse primer.

In some embodiments, a method provided comprises a step of amplifying a (wild-type) nucleic acid molecule. In certain embodiments, amplification of these targets comprises use of primers that comprise sequences complementary to the sequence of a portion of the nucleic acid molecule of interest.

In some embodiments, a primer provided herein comprises or consists of the following parts: (1) P5 or P7—this is the sequence that binds to the Illumina flowcell and is defined by Illumina, wherein forward/i5 primers use P5 and reverse/i7 primers use P7; (2) DNA barcode (e.g., index sequence); (3) Illumina priming sequence, TruSeq type—defined by Illumina, this is where primers bind; (4) diversity spacer—0 to 3 bases to shift the register of the sequence downstream so that in any given cycle there is more diversity than if no spacer was employed, and any given barcode is assigned a specific spacer, as Illumina reportedly sequences in lockstep, first base 1 of all clusters, then base 2 and so forth; (5) the priming sequence, which corresponds to a nucleic acid sequence of interest or its complement.

In some embodiments, a primer includes (a) a block of 12 nucleotides corresponding to the appropriate DNA barcode and (b) a diversity spacer comprising 0 to 3 bases, wherein sequences (a) and (b) are both 5′ to the targeting sequence, in order to increase the base diversity at each sequencing position and improve the quality of base calling; and each barcode is paired with a specific spacer length.

In some embodiments, a primer for use in a method of the disclosure has a structure corresponding to that of a universal primer, such as:

NEBnext Universal primer
(SEQ ID NO: 1)
5′-AATGATACGGCGACCACCGAGATCTACACTCTTTCCCTACACGACGC
TCTTCCGATCT-3′
Universal Primer [Tm 75deg]
(SEQ ID NO: 1)
5′-AATGATACGGCGACCACCGAGATCTACACTCTTTCCCTACACGACGC
TCTTCCGATCT-3′

wherein, in a primer for use in a method of the disclosure, a unique DNA barcode is inserted in the middle, and the sequence complementary to the sequence of a nucleic acid molecule of interest is added at the 3′ end.

For example, the S2 i5 primer designated “S2-i5t0-TGTTCTTCGTAA” (SEQ ID NO: 2) comprises a DNA bar code sequence which is 5′-TGTTCTTCGTAA-3′ (SEQ ID NO: 2) and no spacer (the spacer length is zero), and has a sequence of: 5′-AATGATACGGCGACCACCGAGATCTACAC TGTTCTTCGTAA ACACTCTTTCCCTACACGACGCTCTTCCGATCT XXXXXXXXXXXXXXXXXXXX-3′ (SEQ ID NO: 3), wherein the underlined (but not bold) portions correspond to overlapping portions of the universal primers, the bold, underlined portion represents the barcode, and the bold, not underlined portion represents a sequence complementary to that of the nucleic acid of interest (e.g., X can be any suitable nucleotide, and the region of XX . . . XX can be any suitable length).

Table 1 provides unique barcodes for i5 primers; to determine the sequence of a corresponding primer, the sequence 5′-AATGATACGGCGACCACCGAGATCTACAC-3′ (SEQ ID NO: 4) is added at the 5′ end, and the sequence 5′-ACACTCTTTCCCTACACGACGCTCTTCCGATCT XXXXXXXXXXXXXXXXXXXX-3′ (SEQ ID NO: 5), is added at the 3′ end, wherein X can be any suitable nucleotide, and the region of XX . . . XX has a sequence complementary to that of the nucleic acid of interest and can be any suitable length.

In some embodiments, the present disclosure pertains to any primer comprising a barcode sequence provided in Table 1. In some embodiments, the present disclosure pertains to any primer which is useful for a method of the present disclosure which comprises a barcode sequence provided in Table 1.

TABLE 1
Example unique barcodes

| | | SEQ | | | | SEQ | |
|---|---|---|---|---|---|---|---|
| | | ID | | | | ID | |
| | | NO: | | | | NO: | |
| | S2-i5t0-TGTTCTTCGTAA | 2 | S2 | i5 | TGTTCTTCGTAA | 2 | 0 |
| 726 | S2-i5t0-ATTGATTATTCG | 6 | S2 | i5 | ATTGATTATTCG | 6 | 0 |
| 727 | S2-i5t0-GCTCTGCCGGCT | 7 | S2 | i5 | GCTCTGCCGGCT | 7 | 0 |
| 728 | S2-i5t0-GATTCGTTAGCG | 8 | S2 | i5 | GATTCGTTAGCG | 8 | 0 |
| 729 | S2-i5t0- | 9 | S2 | i5 | TACAAGCGAGGT | 9 | 0 |
| | TACAAGCGAGGT | | | | | | |
| 730 | S2-i5t0-TTCCAGGTCGCG | 10 | S2 | i5 | TTCCAGGTCGCG | 10 | 0 |
| 731 | S2-i5t0-AGTAACTAGTCG | 11 | S2 | i5 | AGTAACTAGTCG | 11 | 0 |
| 732 | S2-i5t0-AATCTCGAAGTC | 12 | S2 | i5 | AATCTCGAAGTC | 12 | 0 |
| 733 | S2-i5t0-CCGGGAATGTTA | 13 | S2 | i5 | CCGGGAATGTTA | 13 | 0 |
| 734 | S2-i5t0-CCGTTGGATTTG | 14 | S2 | i5 | CCGTTGGATTTG | 14 | 0 |
| 735 | S2-i5t0- | 15 | S2 | i5 | CCACGAGGTGAC | 15 | 0 |
| | CCACGAGGTGAC | | | | | | |
| 736 | S2-i5t0-CGCGATCCATCT | 16 | S2 | i5 | CGCGATCCATCT | 16 | 0 |
| 737 | S2-i5t1-CTCCGTGGGAGT | 17 | S2 | i5 | CTCCGTGGGAGT | 17 | 1 |
| 738 | S2-i5t1- | 18 | S2 | i5 | GCAAGACCCAAT | 18 | 1 |
| | GCAAGACCCAAT | | | | | | |
| 739 | S2-i5t1-TTGTGACCTTAC | 19 | S2 | i5 | TTGTGACCTTAC | 19 | 1 |
| 740 | S2-i5t1-ATACCGATAGTG | 20 | S2 | i5 | ATACCGATAGTG | 20 | 1 |
| 741 | S2-i5t1-TGATCAGCGATC | 21 | S2 | i5 | TGATCAGCGATC | 21 | 1 |
| 742 | S2-i5t1-CCCAGGCTTCTC | 22 | S2 | i5 | CCCAGGCTTCTC | 22 | 1 |
| 743 | S2-i5t1- | 23 | S2 | i5 | CCCAGCGCAATA | 23 | 1 |
| | CCCAGCGCAATA | | | | | | |
| 744 | S2-i5t1-CTACTAGAAATA | 24 | S2 | i5 | CTACTAGAAATA | 24 | 1 |
| 745 | S2-i5t1- | 25 | S2 | i5 | CGGAAGGGCTAC | 25 | 1 |
| | CGGAAGGGCTAC | | | | | | |
| 746 | S2-i5t1-TGGACGGCTGTG | 26 | S2 | i5 | TGGACGGCTGTG | 26 | 1 |
| 747 | S2-i5t1- | 27 | S2 | i5 | GTGCAAGGCACA | 27 | 1 |
| | GTGCAAGGCACA | | | | | | |
| 748 | S2-i5t2-CGAGTCTTGACG | 28 | S2 | i5 | CGAGTCTTGACG | 28 | 2 |
| 749 | S2-i5t2-GATCGCGTTGAG | 29 | S2 | i5 | GATCGCGTTGAG | 29 | 2 |
| 750 | S2-i5t2- | 30 | S2 | i5 | TATGATGCAGGA | 30 | 2 |
| | TATGATGCAGGA | | | | | | |
| 751 | S2-i5t2- | 31 | S2 | i5 | ACAGATAACCAT | 31 | 2 |
| | ACAGATAACCAT | | | | | | |
| 752 | S2-i5t2-GTATGTCTGAAA | 32 | S2 | i5 | GTATGTCTGAAA | 32 | 2 |
| 753 | S2-i5t2-CTCAGCTTTAAA | 33 | S2 | i5 | CTCAGCTTTAAA | 33 | 2 |
| 754 | S2-i5t2-GAATTCTAGGTT | 34 | S2 | i5 | GAATTCTAGGTT | 34 | 2 |
| 755 | S2-i5t2-TGCGCGACCACT | 35 | S2 | i5 | TGCGCGACCACT | 35 | 2 |
| 756 | S2-i5t2-ACACCTTCCGCA | 36 | S2 | i5 | ACACCTTCCGCA | 36 | 2 |
| 757 | S2-i5t2-TATACTCAGGAT | 37 | S2 | i5 | TATACTCAGGAT | 37 | 2 |
| 758 | S2-i5t2-TCAATCCAAACG | 38 | S2 | i5 | TCAATCCAAACG | 38 | 2 |
| 759 | S2-i5t2-ATAGGTGAATGT | 39 | S2 | i5 | ATAGGTGAATGT | 39 | 2 |
| 760 | S2-i5t3-TCCAATTCGACG | 40 | S2 | i5 | TCCAATTCGACG | 40 | 3 |
| 761 | S2-i5t3-CATCGCTGATCA | 41 | S2 | i5 | CATCGCTGATCA | 41 | 3 |
| 762 | S2-i5t3-CGGGTAAATTGT | 42 | S2 | i5 | CGGGTAAATTGT | 42 | 3 |
| 763 | S2-i5t3-CTACTAACCCAG | 43 | S2 | i5 | CTACTAACCCAG | 43 | 3 |
| 764 | S2-i5t3- | 44 | S2 | i5 | GACATCCGAGGA | 44 | 3 |
| | GACATCCGAGGA | | | | | | |
| 765 | S2-i5t3-CCACCTCCTTGT | 45 | S2 | i5 | CCACCTCCTTGT | 45 | 3 |
| 766 | S2-i5t3-ACTTGGAAGTTC | 46 | S2 | i5 | ACTTGGAAGTTC | 46 | 3 |
| 767 | S2-i5t3-TCCAAGCAGCTG | 47 | S2 | i5 | TCCAAGCAGCTG | 47 | 3 |
| 768 | S2-i5t3-ATTCACCTTCGC | 48 | S2 | i5 | ATTCACCTTCGC | 48 | 3 |
| 769 | S2-i5t3-CTCGTTTCCAAC | 49 | S2 | i5 | CTCGTTTCCAAC | 49 | 3 |
| 770 | S2-i5t3- | 50 | S2 | i5 | TCCGAGGGTACA | 50 | 3 |
| | TCCGAGGGTACA | | | | | | |
| 771 | S2-i5t0-GTAATATAGCCT | 51 | S2 | i5 | GTAATATAGCCT | 51 | 0 |
| 772 | S2-i5t0-GTAATAGGTTAT | 52 | S2 | i5 | GTAATAGGTTAT | 52 | 0 |
| 773 | S2-i5t0-CGCATTGTATTT | 53 | S2 | i5 | CGCATTGTATTT | 53 | 0 |
| 774 | S2-i5t0-TCATGGATTACG | 54 | S2 | i5 | TCATGGATTACG | 54 | 0 |
| 775 | S2-i5t0-ACTCACGAAGCT | 55 | S2 | i5 | ACTCACGAAGCT | 55 | 0 |
| 776 | S2-i5t0-CAATTCGCCAAA | 56 | S2 | i5 | CAATTCGCCAAA | 56 | 0 |
| 777 | S2-i5t0-AACATTCTATAC | 57 | S2 | i5 | AACATTCTATAC | 57 | 0 |
| 778 | S2-i5t0- | 58 | S2 | i5 | CAACACTTGAAA | 58 | 0 |
| | CAACACTTGAAA | | | | | | |
| 779 | S2-i5t0-AGAGAGTGTCTC | 59 | S2 | i5 | AGAGAGTGTCTC | 59 | 0 |
| 780 | S2-i5t0-TTCATTTGGCTC | 60 | S2 | i5 | TTCATTTGGCTC | 60 | 0 |
| 781 | S2-i5t1-GCGTTGGTACCG | 61 | S2 | i5 | GCGTTGGTACCG | 61 | 1 |
| 782 | S2-i5t1- | 62 | S2 | i5 | CACTGACGGTGA | 62 | 1 |
| | CACTGACGGTGA | | | | | | |
| 783 | S2-i5t1-CGGTCACTGAAT | 63 | S2 | i5 | CGGTCACTGAAT | 63 | 1 |
| 784 | S2-i5t1-CCTCTGAGATAG | 64 | S2 | i5 | CCTCTGAGATAG | 64 | 1 |
| 785 | S2-i5t1-ATCGCATCATTG | 65 | S2 | i5 | ATCGCATCATTG | 65 | 1 |
| 786 | S2-i5t1-CAGTTTCAGCGC | 66 | S2 | i5 | CAGTTTCAGCGC | 66 | 1 |
| 787 | S2-i5t1-ATCGGTTATATT | 67 | S2 | i5 | ATCGGTTATATT | 67 | 1 |
| 788 | S2-i5t1- | 68 | S2 | i5 | TCGGGAGACGCT | 68 | 1 |
| | TCGGGAGACGCT | | | | | | |
| 789 | S2-i5t1-GCTTCTTTATTT | 69 | S2 | i5 | GCTTCTTTATTT | 69 | 1 |
| 790 | S2-i5t1- | 70 | S2 | i5 | CAGCCCAAGAGC | 70 | 1 |
| | CAGCCCAAGAGC | | | | | | |
| 791 | S2-i5t1-CGTATTTCCGAT | 71 | S2 | i5 | CGTATTTCCGAT | 71 | 1 |
| 792 | S2-i5t2-AGGAATGTCTGT | 72 | S2 | i5 | AGGAATGTCTGT | 72 | 2 |
| 793 | S2-i5t2-TTACGCTTGACT | 73 | S2 | i5 | TTACGCTTGACT | 73 | 2 |
| 794 | S2-i5t2- | 74 | S2 | i5 | CGAGAAAGGTGT | 74 | 2 |
| | CGAGAAAGGTGT | | | | | | |
| 795 | S2-i5t2-TAATTCAAGCTG | 75 | S2 | i5 | TAATTCAAGCTG | 75 | 2 |
| 796 | S2-i5t2- | 76 | S2 | i5 | TGGACGGTCAAC | 76 | 2 |
| | TGGACGGTCAAC | | | | | | |
| 797 | S2-i5t2- | 77 | S2 | i5 | GTAACGGTGGGT | 77 | 2 |
| | GTAACGGTGGGT | | | | | | |
| 798 | S2-i5t2-ATCACGGCTGGC | 78 | S2 | i5 | ATCACGGCTGGC | 78 | 2 |
| 799 | S2-i5t2-TAACATTCGCGT | 79 | S2 | i5 | TAACATTCGCGT | 79 | 2 |
| 800 | S2-i5t2-ATCTGGTTATGC | 80 | S2 | i5 | ATCTGGTTATGC | 80 | 2 |
| 801 | S2-i5t2-GCGCTACCCAGC | 81 | S2 | i5 | GCGCTACCCAGC | 81 | 2 |
| 802 | S2-i5t2- | 82 | S2 | i5 | AGCGTGGGAGTT | 82 | 2 |
| | AGCGTGGGAGTT | | | | | | |
| 803 | S2-i5t2- | 83 | S2 | i5 | TAGAAGGAGCTA | 83 | 2 |
| | TAGAAGGAGCTA | | | | | | |
| 804 | S2-i5t3-TTCGTACAGCTC | 84 | S2 | i5 | TTCGTACAGCTC | 84 | 3 |
| 805 | S2-i5t3- | 85 | S2 | i5 | TTTAGGACGAGA | 85 | 3 |
| | TTTAGGACGAGA | | | | | | |
| 806 | S2-i5t3- | 86 | S2 | i5 | TTGGCAGAACGA | 86 | 3 |
| | TTGGCAGAACGA | | | | | | |
| 807 | S2-i5t3-ACATGACTCATT | 87 | S2 | i5 | ACATGACTCATT | 87 | 3 |
| 808 | S2-i5t3-CCAGTTCGATGA | 88 | S2 | i5 | CCAGTTCGATGA | 88 | 3 |
| 809 | S2-i5t3-TCCGTACGTCGA | 89 | S2 | i5 | TCCGTACGTCGA | 89 | 3 |
| 810 | S2-i5t3-TCGGCTGACATC | 90 | S2 | i5 | TCGGCTGACATC | 90 | 3 |
| 811 | S2-i5t3-CGTTGGGACTTT | 91 | S2 | i5 | CGTTGGGACTTT | 91 | 3 |
| 812 | S2-i5t3-TGCATCTGGATA | 92 | S2 | i5 | TGCATCTGGATA | 92 | 3 |
| 813 | S2-i5t3-TGGCCAAACCTT | 93 | S2 | i5 | TGGCCAAACCTT | 93 | 3 |
| 814 | S2-i5t3-TCTGCTGCCTTG | 94 | S2 | i5 | TCTGCTGCCTTG | 94 | 3 |
| 815 | S2-i5t3-CTCAGGATAACT | 195 | S2 | i5 | CTCAGGATAACT | 95 | 3 |
| 816 | S2-i5t0- | 96 | S2 | i5 | TGGCAAGGAAAG | 96 | 0 |
| | TGGCAAGGAAAG | | | | | | |
| 817 | S2-i5t0-ACCCGAAGTTTA | 197 | S2 | i5 | ACCCGAAGTTTA | 97 | 0 |
| 818 | S2-i5t0-AGTAAACTGCTA | 98 | S2 | i5 | AGTAAACTGCTA | 98 | 0 |
| 819 | S2-i5t0-TTGCCCATAGGT | 199 | S2 | i5 | TTGCCCATAGGT | 199 | 0 |
| 820 | S2-i5t0-AGTGGTAGTTAG | 100 | S2 | i5 | AGTGGTAGTTAG | 100 | 0 |
| 821 | S2-i5t0- | 101 | S2 | i5 | CAATAAACGCCG | 101 | 0 |
| | CAATAAACGCCG | | | | | | |
| 822 | S2-i5t0-TTGACCACGACG | 102 | S2 | i5 | TTGACCACGACG | 102 | 0 |
| 823 | S2-i5t0-GCACTCCTAAAT | 103 | S2 | i5 | GCACTCCTAAAT | 103 | 0 |
| 824 | S2-i5t0- | 104 | S2 | i5 | CCAGACATATGA | 104 | 0 |
| | CCAGACATATGA | | | | | | |
| 825 | S2-i5t0-CATTACCCGTCA | 105 | S2 | i5 | CATTACCCGTCA | 105 | 0 |
| 826 | S2-i5t0- | 106 | S2 | i5 | ACGGTTGAGAAT | 106 | 0 |
| | ACGGTTGAGAAT | | | | | | |
| 827 | S2-i5t1-CTACCTCAGAAT | 107 | S2 | i5 | CTACCTCAGAAT | 107 | 1 |
| 828 | S2-i5t1-TTTGTACACCGT | 108 | S2 | i5 | TTTGTACACCGT | 108 | 1 |
| 829 | S2-i5t1- | 109 | S2 | i5 | CCGAACAGGTAA | 109 | 1 |
| | CCGAACAGGTAA | | | | | | |
| 830 | S2-i5t1-TGTCGGATTAAA | 110 | S2 | i5 | TGTCGGATTAAA | 110 | 1 |
| 831 | S2-i5t1-ACGGACCTCCTA | 111 | S2 | i5 | ACGGACCTCCTA | 111 | 1 |
| 832 | S2-i5t1- | 112 | S2 | i5 | TAGATAAGTCAG | 112 | 1 |
| | TAGATAAGTCAG | | | | | | |
| 833 | S2-i5t1-AATTTCGGCTTA | 113 | S2 | i5 | AATTTCGGCTTA | 113 | 1 |
| 834 | S2-i5t1-AATTGTTTGAAG | 114 | S2 | i5 | AATTGTTTGAAG | 114 | 1 |
| 835 | S2-i5t1- | 115 | S2 | i5 | ATGGAGCTGAAG | 115 | 1 |
| | ATGGAGCTGAAG | | | | | | |
| 836 | S2-i5t1-AGCTGTTACCAG | 116 | S2 | i5 | AGCTGTTACCAG | 116 | 1 |
| 837 | S2-i5t1-AGCACCCGATTC | 117 | S2 | i5 | AGCACCCGATTC | 117 | 1 |
| 838 | S2-i5t1-CCTAACCTCCCG | 118 | S2 | i5 | CCTAACCTCCCG | 118 | 1 |
| 839 | S2-i5t2-AGGGCTGTGCTG | 119 | S2 | i5 | AGGGCTGTGCTG | 119 | 2 |
| 840 | S2-i5t2-ACATGCTTTGCG | 120 | S2 | i5 | ACATGCTTTGCG | 120 | 2 |
| 841 | S2-i5t2-TACTGTTGATAT | 121 | S2 | i5 | TACTGTTGATAT | 121 | 2 |
| 842 | S2-i5t2-CGTGCGCACCTA | 122 | S2 | i5 | CGTGCGCACCTA | 122 | 2 |
| 843 | S2-i5t2-GAGATCTTTAGC | 123 | S2 | i5 | GAGATCTTTAGC | 123 | 2 |
| 844 | S2-i5t2- | 124 | S2 | i5 | AATGCAGCGCAA | 124 | 2 |
| | AATGCAGCGCAA | | | | | | |
| 845 | S2-i5t2-TACAATGGTCAT | 125 | S2 | i5 | TACAATGGTCAT | 125 | 2 |
| 846 | S2-i5t2- | 126 | S2 | i5 | TAAGAGACTACA | 126 | 2 |
| | TAAGAGACTACA | | | | | | |
| 847 | S2-i5t2-CTTAAGATGATC | 127 | S2 | i5 | CTTAAGATGATC | 127 | 2 |
| 848 | S2-i5t3-AATTTGCCCATT | 128 | S2 | i5 | AATTTGCCCATT | 128 | 3 |
| 849 | S2-i5t3-TTACTCTGATCA | 129 | S2 | i5 | TTACTCTGATCA | 129 | 3 |
| 850 | S2-i5t3-AGTTGTCTTGTT | 130 | S2 | i5 | AGTTGTCTTGTT | 130 | 3 |
| 851 | S2-i5t3- | 131 | S2 | i5 | GAGTGGAATGAC | 131 | 3 |
| | GAGTGGAATGAC | | | | | | |
| 852 | S2-i5t3-GTCCGCTTGCAA | 132 | S2 | i5 | GTCCGCTTGCAA | 132 | 3 |
| 853 | S2-i5t3-GTTACGTGGTCT | 133 | S2 | i5 | GTTACGTGGTCT | 133 | 3 |
| 854 | S2-i5t3-GATCTGTGGCTT | 134 | S2 | i5 | GATCTGTGGCTT | 134 | 3 |
| 855 | S2-i5t3-CTAAAGTCTGTA | 135 | S2 | i5 | CTAAAGTCTGTA | 135 | 3 |
| 856 | S2-i5t3-TCGTACCGGTCT | 136 | S2 | i5 | TCGTACCGGTCT | 136 | 3 |
| 857 | S2-i5t3-GCCCACTCAGAC | 137 | S2 | i5 | GCCCACTCAGAC | 137 | 3 |
| 858 | S2-i5t3-AGGATAGTCATC | 138 | S2 | i5 | AGGATAGTCATC | 138 | 3 |
| 859 | S2-i5t0-ATTGCTCGGGCA | 139 | S2 | i5 | ATTGCTCGGGCA | 139 | 0 |
| 860 | S2-i5t0-CACAGACGTTAC | 140 | S2 | i5 | CACAGACGTTAC | 140 | 0 |
| 861 | S2-i5t0- | 141 | S2 | i5 | CCAGGGAATAAG | 141 | 0 |
| | CCAGGGAATAAG | | | | | | |
| 862 | S2-i5t0-TTCCCGAACTAC | 142 | S2 | i5 | TTCCCGAACTAC | 142 | 0 |
| 863 | S2-i5t0-AACGAGCCATTT | 143 | S2 | i5 | AACGAGCCATTT | 143 | 0 |
| 864 | S2-i5t0-GCGCGTCCTTTC | 144 | S2 | i5 | GCGCGTCCTTTC | 144 | 0 |
| 865 | S2-i5t0-TCTATCGTGTTC | 145 | S2 | i5 | TCTATCGTGTTC | 145 | 0 |
| 866 | S2-i5t0- | 146 | S2 | i5 | AAACGCTAAGGA | 146 | 0 |
| | AAACGCTAAGGA | | | | | | |
| 867 | S2-i5t0-TGACACAGATCT | 147 | S2 | i5 | TGACACAGATCT | 147 | 0 |
| 868 | S2-i5t0-CCCTACCTCTCA | 148 | S2 | i5 | CCCTACCTCTCA | 148 | 0 |
| 869 | S2-i5t0-TGCGGATCCGCA | 149 | S2 | i5 | TGCGGATCCGCA | 149 | 0 |
| 870 | S2-i5t0- | 150 | S2 | i5 | GTCAACAACGAC | 150 | 0 |
| | GTCAACAACGAC | | | | | | |
| 871 | S2-i5t1- | 151 | S2 | i5 | AGGACCACCCAG | 151 | 1 |
| | AGGACCACCCAG | | | | | | |
| 872 | S2-i5t1-CCAAGTACATTC | 152 | S2 | i5 | CCAAGTACATTC | 152 | 1 |
| 873 | S2-i5t1-GTGCTGTTTCCT | 153 | S2 | i5 | GTGCTGTTTCCT | 153 | 1 |
| 874 | S2-i5t1- | 154 | S2 | i5 | CAAATGCTAGAA | 154 | 1 |
| | CAAATGCTAGAA | | | | | | |
| 875 | S2-i5t1- | 155 | S2 | i5 | ATCATACAAGGA | 155 | 1 |
| | ATCATACAAGGA | | | | | | |
| 876 | S2-i5t1- | 156 | S2 | i5 | CATAGTAAGTAG | 156 | 1 |
| | CATAGTAAGTAG | | | | | | |
| 877 | S2-i5t1- | 157 | S2 | i5 | CTTAATAAAGAG | 157 | 1 |
| | CTTAATAAAGAG | | | | | | |
| 878 | S2-i5t1-GTACGCTCACCG | 158 | S2 | i5 | GTACGCTCACCG | 158 | 1 |
| 879 | S2-i5t1-CCCGATAAATGT | 159 | S2 | i5 | CCCGATAAATGT | 159 | 1 |
| 880 | S2-i5t1-ATCGCTTAATAC | 160 | S2 | i5 | ATCGCTTAATAC | 160 | 1 |
| 881 | S2-i5t1-TTGACGCATTAC | 161 | S2 | i5 | TTGACGCATTAC | 161 | 1 |
| 882 | S2-i5t1- | 162 | S2 | i5 | TTAGTAGGCGAG | 162 | 1 |
| | TTAGTAGGCGAG | | | | | | |
| 883 | S2-i5t2-GTTCACTCACAA | 163 | S2 | i5 | GTTCACTCACAA | 163 | 2 |
| 884 | S2-i5t2-CATCGGGCCATG | 164 | S2 | i5 | CATCGGGCCATG | 164 | 2 |
| 885 | S2-i5t2-AATTACCCAGCT | 165 | S2 | i5 | AATTACCCAGCT | 165 | 2 |
| 886 | S2-i5t2-CGCATCAATTCG | 166 | S2 | i5 | CGCATCAATTCG | 166 | 2 |
| 887 | S2-i5t2-TCAACCCGTCAT | 167 | S2 | i5 | TCAACCCGTCAT | 167 | 2 |
| 888 | S2-i5t2-ACTAGTTCGAGT | 168 | S2 | i5 | ACTAGTTCGAGT | 168 | 2 |
| 889 | S2-i5t2- | 169 | S2 | 15 | TACAGGACGCTG | 169 | 2 |
| | TACAGGACGCTG | | | | | | |
| 890 | S2-i5t2-AATGGGCTCGAT | 170 | S2 | i5 | AATGGGCTCGAT | 170 | 2 |
| 891 | S2-i5t2-CACCTTGACAAC | 171 | S2 | i5 | CACCTTGACAAC | 171 | 2 |
| 892 | S2-i5t2-ACTCACCACTGC | 172 | S2 | i5 | ACTCACCACTGC | 172 | 2 |
| 893 | S2-i5t2-TAGTTCACACAG | 173 | S2 | i5 | TAGTTCACACAG | 173 | 2 |
| 894 | S2-i5t3-CTTGCGTTGAGT | 174 | S2 | i5 | CTTGCGTTGAGT | 174 | 3 |
| 895 | S2-i5t3-GCTGATCCCATC | 175 | S2 | i5 | GCTGATCCCATC | 175 | 3 |
| 896 | S2-i5t3-TGAGTTCTAGAT | 176 | S2 | i5 | TGAGTTCTAGAT | 176 | 3 |
| 897 | S2-i5t3-CAATCTCCATCT | 177 | S2 | i5 | CAATCTCCATCT | 177 | 3 |
| 898 | S2-i5t3- | 178 | S2 | i5 | AGGCAAAGCAGA | 178 | 3 |
| | AGGCAAAGCAGA | | | | | | |
| 899 | S2-i5t3-CATGTTAACGCA | 179 | S2 | i5 | CATGTTAACGCA | 179 | 3 |
| 900 | S2-i5t3-TGCCGATTACCA | 180 | S2 | i5 | TGCCGATTACCA | 180 | 3 |
| 901 | S2-i5t3-GTGACCTATCCA | 181 | S2 | i5 | GTGACCTATCCA | 181 | 3 |
| 902 | S2-i5t3- | 182 | S2 | i5 | AGTGGTAAGTGC | 182 | 3 |
| | AGTGGTAAGTGC | | | | | | |
| 903 | S2-i5t3- | 183 | S2 | i5 | AGGGAACATCGT | 183 | 3 |
| | AGGGAACATCGT | | | | | | |
| 904 | S2-i5t0-CGCCATGACTTC | 184 | S2 | i5 | CGCCATGACTTC | 184 | 0 |
| 905 | S2-i5t0-ATCTCGCCAAAC | 185 | S2 | i5 | ATCTCGCCAAAC | 185 | 0 |
| 906 | S2-i5t0-CGCCTGGCCGTA | 186 | S2 | i5 | CGCCTGGCCGTA | 186 | 0 |
| 907 | S2-i5t0-AGGGTGCTGTCG | 187 | S2 | i5 | AGGGTGCTGTCG | 187 | 0 |
| 908 | S2-i5t0-AGCCTCCGCCCT | 188 | S2 | i5 | AGCCTCCGCCCT | 188 | 0 |
| 909 | S2-i5t0-GTTTAATCCAAG | 189 | S2 | i5 | GTTTAATCCAAG | 189 | 0 |
| 910 | S2-i5t0-CAGCTATGACCT | 190 | S2 | i5 | CAGCTATGACCT | 190 | 0 |
| 911 | S2-i5t0-ACGACTGGCACT | 191 | S2 | i5 | ACGACTGGCACT | 191 | 0 |
| 912 | S2-i5t0-GTCTCTCGTTGT | 192 | S2 | i5 | GTCTCTCGTTGT | 192 | 0 |
| 913 | S2-i5t0-TAAACGCGTTTG | 193 | S2 | i5 | TAAACGCGTTTG | 193 | 0 |
| 914 | S2-i5t0- | 194 | S2 | i5 | GTGCCACGAAAC | 194 | 0 |
| | GTGCCACGAAAC | | | | | | |
| 915 | S2-i5t0-CAATTGCACCAT | 195 | S2 | i5 | CAATTGCACCAT | 195 | 0 |
| 916 | S2-i5t1- | 196 | S2 | i5 | CGGTACGGAGTC | 196 | 1 |
| | CGGTACGGAGTC | | | | | | |
| 917 | S2-i5t1-TTCTCTAGGAAC | 197 | S2 | i5 | TTCTCTAGGAAC | 197 | 1 |
| 918 | S2-i5t1-AACGTCGAATTA | 198 | S2 | i5 | AACGTCGAATTA | 198 | 1 |
| 919 | S2-i5t1-TTGTCCGGAACT | 199 | S2 | i5 | TTGTCCGGAACT | 199 | 1 |
| 920 | S2-i5t1-ATGCCTGTCCAA | 200 | S2 | i5 | ATGCCTGTCCAA | 200 | 1 |
| 921 | S2-i5t1- | 201 | S2 | i5 | TGAAGCACCAAT | 201 | 1 |
| | TGAAGCACCAAT | | | | | | |
| 922 | S2-i5t1-AAGGATCCTCTT | 202 | S2 | i5 | AAGGATCCTCTT | 202 | 1 |
| 923 | S2-i5t1-CTTCATGTTAAA | 203 | S2 | i5 | CTTCATGTTAAA | 203 | 1 |
| 924 | S2-i5t1-TTGAGCCACCTC | 204 | S2 | i5 | TTGAGCCACCTC | 204 | 1 |
| 925 | S2-i5t1-GCTTATGTTGAC | 205 | S2 | i5 | GCTTATGTTGAC | 205 | 1 |
| 926 | S2-i5t1-CGACGATGGGTT | 206 | S2 | i5 | CGACGATGGGTT | 206 | 1 |
| 927 | S2-i5t1-CGTTCGCTATAT | 207 | S2 | i5 | CGTTCGCTATAT | 207 | 1 |
| 928 | S2-i5t2-CCAGTTGCTCAG | 208 | S2 | i5 | CCAGTTGCTCAG | 208 | 2 |
| 929 | S2-i5t2-CCCGGTCTCAAT | 209 | S2 | i5 | CCCGGTCTCAAT | 209 | 2 |
| 930 | S2-i5t2-GTTTGGATCACG | 210 | S2 | i5 | GTTTGGATCACG | 210 | 2 |
| 931 | S2-i5t2-TTGCCTTCGTAC | 211 | S2 | i5 | TTGCCTTCGTAC | 211 | 2 |
| 932 | S2-i5t2-AGTAGTGTCCAC | 212 | S2 | i5 | AGTAGTGTCCAC | 212 | 2 |
| 933 | S2-i5t2-AGAATTTATCCA | 213 | S2 | i5 | AGAATTTATCCA | 213 | 2 |
| 934 | S2-i5t2- | 214 | S2 | i5 | AATGGATGCACG | 214 | 2 |
| | AATGGATGCACG | | | | | | |
| 935 | S2-i5t2-ATCCAAGGATTG | 215 | S2 | i5 | ATCCAAGGATTG | 215 | 2 |
| 936 | S2-i5t2-TAGTCGTTAGAT | 216 | S2 | i5 | TAGTCGTTAGAT | 216 | 2 |
| 937 | S2-i5t2-TGATACTGAGTA | 217 | S2 | i5 | TGATACTGAGTA | 217 | 2 |
| 938 | S2-i5t2- | 218 | S2 | i5 | GCCTAGTAAAGA | 218 | 2 |
| | GCCTAGTAAAGA | | | | | | |
| 939 | S2-i5t3-GCCTACTTATAG | 219 | S2 | i5 | GCCTACTTATAG | 219 | 3 |
| 940 | S2-i5t3-CCTGCCTTGGCT | 220 | S2 | i5 | CCTGCCTTGGCT | 220 | 3 |
| 941 | S2-i5t3- | 221 | S2 | i5 | GATGGACACAGA | 221 | 3 |
| | GATGGACACAGA | | | | | | |
| 942 | S2-i5t3- | 222 | S2 | i5 | CGCCGGGTTGGA | 222 | 3 |
| | CGCCGGGTTGGA | | | | | | |
| 943 | S2-i5t3-GTCTCCAAGATG | 223 | S2 | i5 | GTCTCCAAGATG | 223 | 3 |
| 944 | S2-i5t3- | 224 | S2 | i5 | AAAGCGTATCAA | 224 | 3 |
| | AAAGCGTATCAA | | | | | | |
| 945 | S2-i5t3- | 225 | S2 | i5 | GCACCGCAGCGT | 225 | 3 |
| | GCACCGCAGCGT | | | | | | |
| 946 | S2-i5t3-TGCGCTGAATGC | 226 | S2 | i5 | TGCGCTGAATGC | 226 | 3 |
| 947 | S2-i5t3-TTGTTCAGAGTC | 227 | S2 | i5 | TTGTTCAGAGTC | 227 | 3 |
| 948 | S2-i5t3- | 228 | S2 | i5 | GAGAATGACTGA | 228 | 3 |
| | GAGAATGACTGA | | | | | | |
| 949 | S2-i5t3- | 229 | S2 | i5 | AAGCGAAGCGAC | 229 | 3 |
| | AAGCGAAGCGAC | | | | | | |
| 950 | S2-i5t3-AGCGTGTTGTAA | 230 | S2 | i5 | AGCGTGTTGTAA | 230 | 3 |
| 951 | S2-i5t0-CCACTATACCTT | 231 | S2 | i5 | CCACTATACCTT | 231 | 0 |
| 952 | S2-i5t0-TGTTAAACTCTG | 232 | S2 | i5 | TGTTAAACTCTG | 232 | 0 |
| 953 | S2-i5t0-ATTTGTAGGGCG | 233 | S2 | i5 | ATTTGTAGGGCG | 233 | 0 |
| 954 | S2-i5t0- | 234 | S2 | i5 | CAGGAATCATAG | 234 | 0 |
| | CAGGAATCATAG | | | | | | |
| 955 | S2-i5t0-ACTGGATAGCAT | 235 | S2 | i5 | ACTGGATAGCAT | 235 | 0 |
| 956 | S2-i5t0- | 236 | S2 | i5 | GCACGTAACACG | 236 | 0 |
| | GCACGTAACACG | | | | | | |
| 957 | S2-i5t0-AACTGCCACCTG | 237 | S2 | i5 | AACTGCCACCTG | 237 | 0 |
| 958 | S2-i5t0- | 238 | S2 | i5 | AGCAACCTAAGT | 238 | 0 |
| | AGCAACCTAAGT | | | | | | |
| 959 | S2-i5t0- | 239 | S2 | i5 | GACTGCTAACAG | 239 | 0 |
| | GACTGCTAACAG | | | | | | |
| 960 | S2-i5t0-TTATAGTTCGCT | 240 | S2 | i5 | TTATAGTTCGCT | 240 | 0 |
| 961 | S2-i5t0-TCGCCACACACA | 241 | S2 | i5 | TCGCCACACACA | 241 | 0 |
| 962 | S2-i5t1-ATAACCACCAGT | 242 | S2 | i5 | ATAACCACCAGT | 242 | 1 |
| 963 | S2-i5t1- | 243 | S2 | i5 | GAATGGGTCCGC | 243 | 1 |
| | GAATGGGTCCGC | | | | | | |
| 964 | S2-i5t1-GTGACTATGGCT | 244 | S2 | i5 | GTGACTATGGCT | 244 | 1 |
| 965 | S2-i5t1-TATAGTCCTGAG | 245 | S2 | i5 | TATAGTCCTGAG | 245 | 1 |
| 966 | S2-i5t1-TGTACACATCAC | 246 | S2 | i5 | TGTACACATCAC | 246 | 1 |
| 967 | S2-i5t1-TACCTTAATGAC | 247 | S2 | i5 | TACCTTAATGAC | 247 | 1 |
| 968 | S2-i5t1- | 248 | S2 | i5 | GCCACAAATTAA | 248 | 1 |
| | GCCACAAATTAA | | | | | | |
| 969 | S2-i5t1- | 249 | S2 | i5 | ACGTTCGGGACG | 249 | 1 |
| | ACGTTCGGGACG | | | | | | |
| 970 | S2-i5t1-TTACCGAACCCA | 250 | S2 | i5 | TTACCGAACCCA | 250 | 1 |
| 971 | S2-i5t1-GTGTCATATTAA | 251 | S2 | i5 | GTGTCATATTAA | 251 | 1 |
| 972 | S2-i5t1- | 252 | S2 | i5 | TGAAGCGACGAG | 252 | 1 |
| | TGAAGCGACGAG | | | | | | |
| 973 | S2-i5t1-ATGCCATGCCGC | 253 | S2 | i5 | ATGCCATGCCGC | 253 | 1 |
| 974 | S2-i5t2-TTGGCCTCGATT | 254 | S2 | i5 | TTGGCCTCGATT | 254 | 2 |
| 975 | S2-i5t2-ATCCTCACTTCA | 255 | S2 | i5 | ATCCTCACTTCA | 255 | 2 |
| 976 | S2-i5t2-AGATTACTTGGA | 256 | S2 | i5 | AGATTACTTGGA | 256 | 2 |
| 977 | S2-i5t2-TAATATACTGGT | 257 | S2 | i5 | TAATATACTGGT | 257 | 2 |
| 978 | S2-i5t2- | 258 | S2 | i5 | TCATGCGGAAAG | 258 | 2 |
| | TCATGCGGAAAG | | | | | | |
| 979 | S2-i5t2- | 259 | S2 | i5 | GAAGGTCCGCAA | 259 | 2 |
| | GAAGGTCCGCAA | | | | | | |
| 980 | S2-i5t2- | 260 | S2 | i5 | ACGGTGATGGAA | 260 | 2 |
| | ACGGTGATGGAA | | | | | | |
| 981 | S2-i5t2-GACCTAACCGGT | 261 | S2 | i5 | GACCTAACCGGT | 261 | 2 |
| 982 | S2-i5t2-TTAGCGACAGCT | 262 | S2 | i5 | TTAGCGACAGCT | 262 | 2 |
| 983 | S2-i5t2-TTGCCACCAATT | 263 | S2 | i5 | TTGCCACCAATT | 263 | 2 |
| 984 | S2-i5t2-CTATCTTGAGAT | 264 | S2 | i5 | CTATCTTGAGAT | 264 | 2 |
| 985 | S2-i5t3-ATTTCATTCCTA | 265 | S2 | i5 | ATTTCATTCCTA | 265 | 3 |
| 986 | S2-i5t3-CGTGGCGCATCG | 266 | S2 | i5 | CGTGGCGCATCG | 266 | 3 |
| 987 | S2-i5t3-CCATTAATTGTC | 267 | S2 | i5 | CCATTAATTGTC | 267 | 3 |
| 988 | S2-i5t3-AAGTTTCTTCAT | 268 | S2 | i5 | AAGTTTCTTCAT | 268 | 3 |
| 989 | S2-i5t3- | 269 | S2 | i5 | TCGTAGAGAACA | 269 | 3 |
| | TCGTAGAGAACA | | | | | | |
| 990 | S2-i5t3- | 270 | S2 | i5 | ACAAGGTTGATG | 270 | 3 |
| | ACAAGGTTGATG | | | | | | |
| 991 | S2-i5t3-GTCTTTACATCT | 271 | S2 | i5 | GTCTTTACATCT | 271 | 3 |
| 992 | S2-i5t3-CTTCGCCGCGTG | 272 | S2 | i5 | CTTCGCCGCGTG | 272 | 3 |
| 993 | S2-i5t3- | 273 | S2 | i5 | TAAGACTACAAG | 273 | 3 |
| | TAAGACTACAAG | | | | | | |
| 994 | S2-i5t0-CTGTCGAAGCCG | 274 | S2 | i5 | CTGTCGAAGCCG | 274 | 0 |
| 995 | S2-i5t0- | 275 | S2 | i5 | TAAGCCCAGGAA | 275 | 0 |
| | TAAGCCCAGGAA | | | | | | |
| 996 | S2-i5t0-TATTGCCGTTTC | 276 | S2 | i5 | TATTGCCGTTTC | 276 | 0 |
| 997 | S2-i5t0-TACCGTCTCTAA | 277 | S2 | i5 | TACCGTCTCTAA | 277 | 0 |
| 998 | S2-i5t0- | 278 | S2 | i5 | GACCATTAGAGC | 278 | 0 |
| | GACCATTAGAGC | | | | | | |
| 999 | S2-i5t0-CATCGACGCTCG | 279 | S2 | i5 | CATCGACGCTCG | 279 | 0 |
| 1000 | S2-i5t0-TTGCCCGACTCA | 280 | S2 | i5 | TTGCCCGACTCA | 280 | 0 |
| 1001 | S2-i5t0-ACGTACGCTAGT | 281 | S2 | i5 | ACGTACGCTAGT | 281 | 0 |
| 1002 | S2-i5t0-CATAGACAACTT | 282 | S2 | i5 | CATAGACAACTT | 282 | 0 |
| 1003 | S2-i5t0- | 283 | S2 | i5 | TGAAGATAGTCG | 283 | 0 |
| | TGAAGATAGTCG | | | | | | |
| 1004 | S2-i5t0- | 284 | S2 | i5 | TAAGATAAGATG | 284 | 0 |
| | TAAGATAAGATG | | | | | | |
| 1005 | S2-i5t0- | 285 | S2 | i5 | AATAGCGAGCTC | 285 | 0 |
| | AATAGCGAGCTC | | | | | | |
| 1006 | S2-i5t1- | 286 | S2 | i5 | ACCGGCGATGGT | 286 | 1 |
| | ACCGGCGATGGT | | | | | | |
| 1007 | S2-i5t1-AGTTCTGGCAAA | 287 | S2 | i5 | AGTTCTGGCAAA | 287 | 1 |
| 1008 | S2-i5t1-TCCCTGCGCGAC | 288 | S2 | i5 | TCCCTGCGCGAC | 288 | 1 |
| 1009 | S2-i5t1-AATATAGCCTCT | 289 | S2 | i5 | AATATAGCCTCT | 289 | 1 |
| 1010 | S2-i5t1-TTTCCGCTACAG | 290 | S2 | i5 | TTTCCGCTACAG | 290 | 1 |
| 1011 | S2-i5t1-GCTTAGATCCAT | 291 | S2 | i5 | GCTTAGATCCAT | 291 | 1 |
| 1012 | S2-i5t1-GCTCTCGAACGT | 292 | S2 | i5 | GCTCTCGAACGT | 292 | 1 |
| 1013 | S2-i5t1-GATACCGATTAC | 293 | S2 | i5 | GATACCGATTAC | 293 | 1 |
| 1014 | S2-i5t1-ACGAACTCCTGT | 294 | S2 | i5 | ACGAACTCCTGT | 294 | 1 |
| 1015 | S2-i5t1-ATAAACGTCGGT | 295 | S2 | i5 | ATAAACGTCGGT | 295 | 1 |
| 1016 | S2-i5t1-GACGTACTCCAA | 296 | S2 | i5 | GACGTACTCCAA | 296 | 1 |
| 1017 | S2-i5t1- | 297 | S2 | i5 | GCAAGCTCGAGA | 297 | 1 |
| | GCAAGCTCGAGA | | | | | | |
| 1018 | S2-i5t2-TCGTGCTTCTTA | 298 | S2 | i5 | TCGTGCTTCTTA | 298 | 2 |
| 1019 | S2-i5t2- | 299 | S2 | i5 | ATCCGAACCAAG | 299 | 2 |
| | ATCCGAACCAAG | | | | | | |
| 1020 | S2-i5t2-GAACATCATTGA | 300 | S2 | i5 | GAACATCATTGA | 300 | 2 |
| 1021 | S2-i5t2-TAAGCCCTAGTT | 301 | S2 | i5 | TAAGCCCTAGTT | 301 | 2 |
| 1022 | S2-i5t2-TGACCACGGTTC | 302 | S2 | i5 | TGACCACGGTTC | 302 | 2 |
| 1023 | S2-i5t2- | 303 | S2 | i5 | CCAATGGAGAAG | 303 | 2 |
| | CCAATGGAGAAG | | | | | | |
| 1024 | S2-i5t2- | 304 | S2 | i5 | GTTGGGCCAGCA | 304 | 2 |
| | GTTGGGCCAGCA | | | | | | |
| 1025 | S2-i5t2-ACTCGATTTACG | 305 | S2 | i5 | ACTCGATTTACG | 305 | 2 |
| 1026 | S2-i5t2-AGCCGTTGCTGT | 306 | S2 | i5 | AGCCGTTGCTGT | 306 | 2 |
| 1027 | S2-i5t2-GTCTTCGAAAGA | 307 | S2 | i5 | GTCTTCGAAAGA | 307 | 2 |
| 1028 | S2-i5t2-ACGTCTGGTGAT | 308 | S2 | i5 | ACGTCTGGTGAT | 308 | 2 |
| 1029 | S2-i5t2- | 309 | S2 | i5 | GAATGGCAAACT | 309 | 2 |
| | GAATGGCAAACT | | | | | | |
| 1030 | S2-i5t3-TATGAGAGTTCG | 310 | S2 | i5 | TATGAGAGTTCG | 310 | 3 |
| 1031 | S2-i5t3-CATCTGGAGCTG | 311 | S2 | i5 | CATCTGGAGCTG | 311 | 3 |
| 1032 | S2-i5t3-CCAGCGACTTCT | 312 | S2 | i5 | CCAGCGACTTCT | 312 | 3 |
| 1033 | S2-i5t3-ACTCAATCGACT | 313 | S2 | i5 | ACTCAATCGACT | 313 | 3 |
| 1034 | S2-i5t3-TCCAGCTAACCT | 314 | S2 | i5 | TCCAGCTAACCT | 314 | 3 |
| 1035 | S2-i5t3- | 315 | S2 | i5 | ACTTAACGGGAG | 315 | 3 |
| | ACTTAACGGGAG | | | | | | |
| 1036 | S2-i5t3-GAGCTTCTGGCA | 316 | S2 | i5 | GAGCTTCTGGCA | 316 | 3 |
| 1037 | S2-i5t3-TCATACACGTTC | 317 | S2 | i5 | TCATACACGTTC | 317 | 3 |
| 1038 | S2-i5t3-CCTGTGATTCCA | 318 | S2 | i5 | CCTGTGATTCCA | 318 | 3 |
| 1039 | S2-i5t0-GCTCTTGATGTC | 319 | S2 | i5 | GCTCTTGATGTC | 319 | 0 |
| 1040 | S2-i5t0-CTGATTCCCGGA | 320 | S2 | i5 | CTGATTCCCGGA | 320 | 0 |
| 1041 | S2-i5t0- | 321 | S2 | i5 | AATCATAGCAAC | 321 | 0 |
| | AATCATAGCAAC | | | | | | |
| 1042 | S2-i5t0- | 322 | S2 | i5 | AAGCGTAAACCG | 322 | 0 |
| | AAGCGTAAACCG | | | | | | |
| 1043 | S2-i5t0-CTCTTGAAACCT | 323 | S2 | i5 | CTCTTGAAACCT | 323 | 0 |
| 1044 | S2-i5t0-CTATCAGGCCCA | 324 | S2 | i5 | CTATCAGGCCCA | 324 | 0 |
| 1045 | S2-i5t0- | 325 | S2 | i5 | AAGTGTAACAAC | 325 | 0 |
| | AAGTGTAACAAC | | | | | | |
| 1046 | S2-i5t0-ACATTACTACTG | 326 | S2 | i5 | ACATTACTACTG | 326 | 0 |
| 1047 | S2-i5t0-TCAAGTTTATGC | 327 | S2 | i5 | TCAAGTTTATGC | 327 | 0 |
| 1048 | S2-i5t0-TCGTGTACACAC | 328 | S2 | i5 | TCGTGTACACAC | 328 | 0 |
| 1049 | S2-i5t0-GATAGCTCTGGA | 329 | S2 | i5 | GATAGCTCTGGA | 329 | 0 |
| 1050 | S2-i5t0- | 330 | S2 | i5 | CAAAGTTAAAGC | 330 | 0 |
| | CAAAGTTAAAGC | | | | | | |
| 1051 | S2-i5t1-TGTATACTGAAC | 331 | S2 | i5 | TGTATACTGAAC | 331 | 1 |
| 1052 | S2-i5t1- | 332 | S2 | i5 | AACAAACGGTCT | 332 | 1 |
| | AACAAACGGTCT | | | | | | |
| 1053 | S2-i5t1-TCCGTGCAGGTT | 333 | S2 | i5 | TCCGTGCAGGTT | 333 | 1 |
| 1054 | S2-i5t1- | 334 | S2 | i5 | AGCAAGTTAGGC | 334 | 1 |
| | AGCAAGTTAGGC | | | | | | |
| 1055 | S2-i5t1-CTAGTGAACTAT | 335 | S2 | i5 | CTAGTGAACTAT | 335 | 1 |
| 1056 | S2-i5t1- | 336 | S2 | i5 | ATGTGAATGACA | 336 | 1 |
| | ATGTGAATGACA | | | | | | |
| 1057 | S2-i5t1- | 337 | S2 | i5 | ACGACGCGCGTA | 337 | 1 |
| | ACGACGCGCGTA | | | | | | |
| 1058 | S2-i5t1- | 338 | S2 | i5 | ACGTGCAGGCGA | 338 | 1 |
| | ACGTGCAGGCGA | | | | | | |
| 1059 | S2-i5t1-CAAGTATGCAAT | 339 | S2 | i5 | CAAGTATGCAAT | 339 | 1 |
| 1060 | S2-i5t1-GTACTCCCGTTA | 340 | S2 | i5 | GTACTCCCGTTA | 340 | 1 |
| 1061 | S2-i5t1-AATACAACCTTC | 341 | S2 | i5 | AATACAACCTTC | 341 | 1 |
| 1062 | S2-i5t1-ACCCATTCCCGC | 342 | S2 | i5 | ACCCATTCCCGC | 342 | 1 |
| 1063 | S2-i5t2-TTAGCTAACAAC | 343 | S2 | i5 | TTAGCTAACAAC | 343 | 2 |
| 1064 | S2-i5t2-TGCCGCTTGGAC | 344 | S2 | i5 | TGCCGCTTGGAC | 344 | 2 |
| 1065 | S2-i5t2-TGATAACGTAGT | 345 | S2 | i5 | TGATAACGTAGT | 345 | 2 |
| 1066 | S2-i5t2-TGCCTCGCCTAA | 346 | S2 | i5 | TGCCTCGCCTAA | 346 | 2 |
| 1067 | S2-i5t2-TACTTGCGGCTC | 347 | S2 | i5 | TACTTGCGGCTC | 347 | 2 |
| 1068 | S2-i5t2-TCGTTGTCAGGA | 348 | S2 | i5 | TCGTTGTCAGGA | 348 | 2 |
| 1069 | S2-i5t2-ATCTCAAGCGTT | 349 | S2 | i5 | ATCTCAAGCGTT | 349 | 2 |
| 1070 | S2-i5t2-CCACCTGGTCTT | 350 | S2 | i5 | CCACCTGGTCTT | 350 | 2 |
| 1071 | S2-i5t3- | 351 | S2 | i5 | ACTAAACCACAC | 351 | 3 |
| | ACTAAACCACAC | | | | | | |
| 1072 | S2-i5t3-TTCGGGCTCGAG | 352 | S2 | i5 | TTCGGGCTCGAG | 352 | 3 |
| 1073 | S2-i5t3-AACTCCCTTCGT | 353 | S2 | i5 | AACTCCCTTCGT | 353 | 3 |
| 1074 | S2-i5t3-GTTAATAGAGGT | 354 | S2 | i5 | GTTAATAGAGGT | 354 | 3 |
| 1075 | S2-i5t3-TCGGACTTATAA | 355 | S2 | i5 | TCGGACTTATAA | 355 | 3 |
| 1076 | S2-i5t3-CCGTAAGTATGT | 356 | S2 | i5 | CCGTAAGTATGT | 356 | 3 |
| 1077 | S2-i5t3-CACCCAGAGTTA | 357 | S2 | i5 | CACCCAGAGTTA | 357 | 3 |
| 1078 | S2-i5t3-CTATCCTAGGAC | 358 | S2 | i5 | CTATCCTAGGAC | 358 | 3 |
| 1079 | S2-i5t3-TCTTGCTCGGTG | 359 | S2 | i5 | TCTTGCTCGGTG | 359 | 3 |
| 1080 | S2-i5t3-GCGGAGCTTTGC | 360 | S2 | i5 | GCGGAGCTTTGC | 360 | 3 |

In some embodiments, a primer for use in a method of the disclosure has a structure corresponding to that of a primer, such as:

NEBnext Indexed primer
5′-CAAGCAGAAGACGGCATACGAGAT . . .
GTGACTGGAGTTCAGACGTGTGCTCTTCCGATCT-3′, (SEQ ID NOS
361-362, respectively, in order of appearance),

wherein, in a primer for use in a method of the disclosure, “ . . . ” is replaced with a unique barcode, and a target nucleic acid sequence 5′-XXXXXXXXXX-3′ is added at the 3′ end, wherein X can be any suitable nucleotide, and the region of XX . . . XX has a sequence complementary to that of the nucleic acid of interest and can be any suitable length. For example, the S2 i7 primer designated “S2-i7t0-AATGCTTCTTGT” (SEQ ID NO: 363) comprises a DNA barcode sequence which is 5′-AATGCTTCTTGT-3′ (SEQ ID NO: 363) and no spacer (the spacer length is zero), and has a sequence of 5′-CAAGCAGAAGACGGCATACGAGAT AATGCTTCTTGT GTGACTGGAGTTCAGACGTGTGCTCTTCCGATCT XXXXXXXXXXXXXXXXXXXX-3′ (SEQ ID NO: 364), wherein the underlined (but not bold) portions correspond to portions of the universal primers, the bold, underlined portion represents the barcode, and the bold, not underlined portion represents a sequence complementary to the sequence of the nucleic acid of interest, wherein X can be any suitable nucleotide, and the region of XX . . . XX has a sequence complementary to that of the nucleic acid of interest and can be any suitable length.

Table 2 provides unique barcodes for i7 primers; to determine the sequence of a corresponding primer, the sequence 5′-CAAGCAGAAGACGGCATACGAGAT-3′ (SEQ ID NO: 361) is added at the 5′ end, and the sequence 5′-GTGACTGGAGTTCAGACGTGTGCTCTTCCGATCT XXXXXXXXXXXXXXXXXXXX-3′ (SEQ ID NO: 365) is added at the 3′ end, wherein X can be any suitable nucleotide, and the region of XX . . . XX has a sequence complementary to that of the nucleic acid of interest and can be any suitable length.

In some embodiments, the present disclosure pertains to any primer comprising a barcode sequence provided in Table 2. In some embodiments, the present disclosure pertains to any primer which is useful for a method of the present disclosure which comprises a barcode sequence provided in Table 2.

TABLE 2
Example unique barcodes

| | | SEQ | | | | SEQ | |
|---|---|---|---|---|---|---|---|
| | | ID | | | | ID | |
| | | NO: | | | | NO: | |
| 1081 | S2-i7t0-AATGCTTCTTGT | 363 | S2 | i7 | AATGCTTCTTGT | 363 | 0 |
| 1082 | S2-i7t0-GCTTATTAGTTA | 366 | S2 | i7 | GCTTATTAGTTA | 366 | 0 |
| 1083 | S2-i7t0-TCGGCCGTCTCG | 367 | S2 | i7 | TCGGCCGTCTCG | 367 | 0 |
| 1084 | S2-i7t0-GCGATTGCTTAG | 368 | S2 | i7 | GCGATTGCTTAG | 368 | 0 |
| 1085 | S2-i7t0-TGGAGCGAACAT | 369 | S2 | i7 | TGGAGCGAACAT | 369 | 0 |
| 1086 | S2-i7t0-GCGCTGGACCTT | 370 | S2 | i7 | GCGCTGGACCTT | 370 | 0 |
| 1087 | S2-i7t0-GCTGATCAATGA | 371 | S2 | i7 | GCTGATCAATGA | 371 | 0 |
| 1088 | S2-i7t0-CTGAAGCTCTAA | 372 | S2 | i7 | CTGAAGCTCTAA | 372 | 0 |
| 1089 | S2-i7t0-ATTGTAAGGGCC | 373 | S2 | i7 | ATTGTAAGGGCC | 373 | 0 |
| 1090 | S2-i7t0-GTTTAGGTTGCC | 374 | S2 | i7 | GTTTAGGTTGCC | 374 | 0 |
| 1091 | S2-i7t0-CAGTGGAGCACC | 375 | S2 | i7 | CAGTGGAGCACC | 375 | 0 |
| 1092 | S2-i7t0-TCTACCTAGCGC | 376 | S2 | i7 | TCTACCTAGCGC | 376 | 0 |
| 1093 | S2-i7t0-GTAGCAAGTATG | 377 | S2 | i7 | GTAGCAAGTATG | 377 | 0 |
| 1094 | S2-i7t0-TGAGGGTGCCTC | 378 | S2 | i7 | TGAGGGTGCCTC | 378 | 0 |
| 1095 | S2-i7t0-TAACCCAGAACG | 379 | S2 | i7 | TAACCCAGAACG | 379 | 0 |
| 1096 | S2-i7t0-CATTCCAGTGTT | 380 | S2 | i7 | CATTCCAGTGTT | 380 | 0 |
| 1097 | S2-i7t0-GTGATAGCCATA | 381 | S2 | i7 | GTGATAGCCATA | 381 | 0 |
| 1098 | S2-i7t0-CTAGCGACTAGT | 382 | S2 | i7 | CTAGCGACTAGT | 382 | 0 |
| 1099 | S2-i7t0-CTCTTCGGACCC | 383 | S2 | i7 | CTCTTCGGACCC | 383 | 0 |
| 1100 | S2-i7t0-ATAACGCGACCC | 384 | S2 | i7 | ATAACGCGACCC | 384 | 0 |
| 1101 | S2-i7t0-ATAAAGATCATC | 385 | S2 | i7 | ATAAAGATCATC | 385 | 0 |
| 1102 | S2-i7t0-CATCGGGAAGGC | 386 | S2 | i7 | CATCGGGAAGGC | 386 | 0 |
| 1103 | S2-i7t0-GTGTCGGCAGGT | 387 | S2 | i7 | GTGTCGGCAGGT | 387 | 0 |
| 1104 | S2-i7t0-ACACGGAACGTG | 388 | S2 | i7 | ACACGGAACGTG | 388 | 0 |
| 1105 | S2-i7t0-GCAGTTCTGAGC | 389 | S2 | i7 | GCAGTTCTGAGC | 389 | 0 |
| 1106 | S2-i7t0-GAGTTGCGCTAG | 390 | S2 | i7 | GAGTTGCGCTAG | 390 | 0 |
| 1107 | S2-i7t0-AGGACGTAGTAT | 391 | S2 | i7 | AGGACGTAGTAT | 391 | 0 |
| 1108 | S2-i7t0-TACCAATAGACA | 392 | S2 | i7 | TACCAATAGACA | 392 | 0 |
| 1109 | S2-i7t0-AAAGTCTGTATG | 393 | S2 | i7 | AAAGTCTGTATG | 393 | 0 |
| 1110 | S2-i7t0-AAATTTCGACTC | 394 | S2 | i7 | AAATTTCGACTC | 394 | 0 |
| 1111 | S2-i7t0-TTGGATCTTAAG | 395 | S2 | i7 | TTGGATCTTAAG | 395 | 0 |
| 1112 | S2-i7t0-TCACCAGCGCGT | 396 | S2 | i7 | TCACCAGCGCGT | 396 | 0 |
| 1113 | S2-i7t0-ACGCCTTCCACA | 397 | S2 | i7 | ACGCCTTCCACA | 397 | 0 |
| 1114 | S2-i7t0-TAGGACTCATAT | 398 | S2 | i7 | TAGGACTCATAT | 398 | 0 |
| 1115 | S2-i7t0-GCAAACCTAACT | 399 | S2 | i7 | GCAAACCTAACT | 399 | 0 |
| 1116 | S2-i7t0-TGTAAGTGGATA | 400 | S2 | i7 | TGTAAGTGGATA | 400 | 0 |
| 1117 | S2-i7t0-GCAGCTTAACCT | 401 | S2 | i7 | GCAGCTTAACCT | 401 | 0 |
| 1118 | S2-i7t0-ACTAGTCGCTAC | 402 | S2 | i7 | ACTAGTCGCTAC | 402 | 0 |
| 1119 | S2-i7t0-TGTTAAATGGGC | 403 | S2 | i7 | TGTTAAATGGGC | 403 | 0 |
| 1120 | S2-i7t0-GACCCAATCATC | 404 | S2 | i7 | GACCCAATCATC | 404 | 0 |
| 1121 | S2-i7t0-AGGAGCCTACAG | 405 | S2 | i7 | AGGAGCCTACAG | 405 | 0 |
| 1122 | S2-i7t0-GCTAGGGAGACG | 406 | S2 | i7 | GCTAGGGAGACG | 406 | 0 |
| 1123 | S2-i7t0-TGTTCCTCCACC | 407 | S2 | i7 | TGTTCCTCCACC | 407 | 0 |
| 1124 | S2-i7t0-CTTGAAGGTTCA | 408 | S2 | i7 | CTTGAAGGTTCA | 408 | 0 |
| 1125 | S2-i7t0-GTCGACGAACCT | 409 | S2 | i7 | GTCGACGAACCT | 409 | 0 |
| 1126 | S2-i7t0-CGCTTCCACTTA | 410 | S2 | i7 | CGCTTCCACTTA | 410 | 0 |
| 1127 | S2-i7t0-CAACCTTTGCTC | 411 | S2 | i7 | CAACCTTTGCTC | 411 | 0 |
| 1128 | S2-i7t0-ACATGGGAGCCT | 412 | S2 | i7 | ACATGGGAGCCT | 412 | 0 |
| 1129 | S2-i7t0-TCCGATATAATG | 413 | S2 | i7 | TCCGATATAATG | 413 | 0 |
| 1130 | S2-i7t0-TATTGGATAATG | 414 | S2 | i7 | TATTGGATAATG | 414 | 0 |
| 1131 | S2-i7t0-GTATTCACGGGT | 415 | S2 | i7 | GTATTCACGGGT | 415 | 0 |
| 1132 | S2-i7t0-TTTATGTTACGC | 416 | S2 | i7 | TTTATGTTACGC | 416 | 0 |
| 1133 | S2-i7t0-AGTGATAGATCC | 4i7 | S2 | i7 | AGTGATAGATCC | 417 | 0 |
| 1134 | S2-i7t0-GCATTAGGTACT | 418 | S2 | i7 | GCATTAGGTACT | 418 | 0 |
| 1135 | S2-i7t0-TCGAAGCACTCA | 419 | S2 | i7 | TCGAAGCACTCA | 419 | 0 |
| 1136 | S2-i7t0-AAACCGCTTAAC | 420 | S2 | i7 | AAACCGCTTAAC | 420 | 0 |
| 1137 | S2-i7t0-CATATCTTACAA | 421 | S2 | i7 | CATATCTTACAA | 421 | 0 |
| 1138 | S2-i7t0-AAAGTTCACAAC | 422 | S2 | i7 | AAAGTTCACAAC | 422 | 0 |
| 1139 | S2-i7t0-CTCTGTGAGAGA | 423 | S2 | i7 | CTCTGTGAGAGA | 423 | 0 |
| 1140 | S2-i7t0-CTCGGTTTACTT | 424 | S2 | i7 | CTCGGTTTACTT | 424 | 0 |
| 1141 | S2-i7t0-GCCATGGTTGCG | 425 | S2 | i7 | GCCATGGTTGCG | 425 | 0 |
| 1142 | S2-i7t0-AGTGGCAGTCAC | 426 | S2 | i7 | AGTGGCAGTCAC | 426 | 0 |
| 1143 | S2-i7t0-TAAGTCACTGGC | 427 | S2 | i7 | TAAGTCACTGGC | 427 | 0 |
| 1144 | S2-i7t0-GATAGAGTCTCC | 428 | S2 | i7 | GATAGAGTCTCC | 428 | 0 |
| 1145 | S2-i7t0-GTTACTACGCTA | 429 | S2 | i7 | GTTACTACGCTA | 429 | 0 |
| 1146 | S2-i7t0-CGCGACTTTGAC | 430 | S2 | i7 | CGCGACTTTGAC | 430 | 0 |
| 1147 | S2-i7t0-TTATATTGGCTA | 431 | S2 | i7 | TTATATTGGCTA | 431 | 0 |
| 1148 | S2-i7t0-ACTATTACGTGT | 432 | S2 | i7 | ACTATTACGTGT | 432 | 0 |
| 1149 | S2-i7t0-TCGCAGAGGGCT | 433 | S2 | i7 | TCGCAGAGGGCT | 433 | 0 |
| 1150 | S2-i7t0-TTTATTTCTTCG | 434 | S2 | i7 | TTTATTTCTTCG | 434 | 0 |
| 1151 | S2-i7t0-TAGCCTTTATGC | 435 | S2 | i7 | TAGCCTTTATGC | 435 | 0 |
| 1152 | S2-i7t0-TGTCTGTAAGGA | 436 | S2 | i7 | TGTCTGTAAGGA | 436 | 0 |
| 1153 | S2-i7t0-TCAGTTCGCATT | 437 | S2 | i7 | TCAGTTCGCATT | 437 | 0 |
| 1154 | S2-i7t0-TGTGGAAAGAGC | 438 | S2 | i7 | TGTGGAAAGAGC | 438 | 0 |
| 1155 | S2-i7t0-GTCGAACTTAAT | 439 | S2 | i7 | GTCGAACTTAAT | 439 | 0 |
| 1156 | S2-i7t0-CAACTGGCAGGT | 440 | S2 | i7 | CAACTGGCAGGT | 440 | 0 |
| 1157 | S2-i7t0-TGGGTGGCAATG | 441 | S2 | i7 | TGGGTGGCAATG | 441 | 0 |
| 1158 | S2-i7t0-CGGTCGGCACTA | 442 | S2 | i7 | CGGTCGGCACTA | 442 | 0 |
| 1159 | S2-i7t0-TGCGCTTACAAT | 443 | S2 | i7 | TGCGCTTACAAT | 443 | 0 |
| 1160 | S2-i7t0-CGTATTGGTCTA | 444 | S2 | i7 | CGTATTGGTCTA | 444 | 0 |
| 1161 | S2-i7t0-CGACCCATCGCG | 445 | S2 | i7 | CGACCCATCGCG | 445 | 0 |
| 1162 | S2-i7t0-TTGAGGGTGCGA | 446 | S2 | i7 | TTGAGGGTGCGA | 446 | 0 |
| 1163 | S2-i7t0-ATCGAGGAAGAT | 447 | S2 | i7 | ATCGAGGAAGAT | 447 | 0 |
| 1164 | S2-i7t0-CTCGACATGCTT | 448 | S2 | i7 | CTCGACATGCTT | 448 | 0 |
| 1165 | S2-i7t0-AGAGCAGGATTT | 449 | S2 | i7 | AGAGCAGGATTT | 449 | 0 |
| 1166 | S2-i7t0-AGCAAGACGGTT | 450 | S2 | i7 | AGCAAGACGGTT | 450 | 0 |
| 1167 | S2-i7t0-TTACTCAGTACA | 451 | S2 | i7 | TTACTCAGTACA | 451 | 0 |
| 1168 | S2-i7t0-AGTAGCTTGACC | 452 | S2 | i7 | AGTAGCTTGACC | 452 | 0 |
| 1169 | S2-i7t0-AGCTGCATGCCT | 453 | S2 | i7 | AGCTGCATGCCT | 453 | 0 |
| 1170 | S2-i7t0-CTACAGTCGGCT | 454 | S2 | i7 | CTACAGTCGGCT | 454 | 0 |
| 1171 | S2-i7t0-TTTCAGGGTTGC | 455 | S2 | i7 | TTTCAGGGTTGC | 455 | 0 |
| 1172 | S2-i7t0-ATAGGTCTACGT | 456 | S2 | i7 | ATAGGTCTACGT | 456 | 0 |
| 1173 | S2-i7t0-TTCCAAACCGGT | 457 | S2 | i7 | TTCCAAACCGGT | 457 | 0 |
| 1174 | S2-i7t0-GTTCCGTCGTCT | 458 | S2 | i7 | GTTCCGTCGTCT | 458 | 0 |
| 1175 | S2-i7t0-TCAATAGGACTC | 459 | S2 | i7 | TCAATAGGACTC | 459 | 0 |
| 1176 | S2-i7t0-ATTTGAAGCCCA | 460 | S2 | i7 | ATTTGAAGCCCA | 460 | 0 |
| 1177 | S2-i7t0-ATCGTCAAATGA | 461 | S2 | i7 | ATCGTCAAATGA | 161 | 0 |
| 1178 | S2-i7t0-TGGATACCCGTT | 462 | S2 | i7 | TGGATACCCGTT | 462 | 0 |
| 1179 | S2-i7t0-GATTGATGGTGA | 463 | S2 | i7 | GATTGATGGTGA | 463 | 0 |
| 1180 | S2-i7t0-GCCGCAAATAAC | 464 | S2 | i7 | GCCGCAAATAAC | 464 | 0 |
| 1181 | S2-i7t0-TAAATCCTCACG | 465 | S2 | i7 | TAAATCCTCACG | 465 | 0 |
| 1182 | S2-i7t0-AGTATACAGACC | 466 | S2 | i7 | AGTATACAGACC | 466 | 0 |
| 1183 | S2-i7t0-TCGGATCATGAA | 467 | S2 | i7 | TCGGATCATGAA | 467 | 0 |
| 1184 | S2-i7t0-ACTGCCCATTAC | 468 | S2 | i7 | ACTGCCCATTAC | 468 | 0 |
| 1185 | S2-i7t0-TAAGAGTTGGCA | 469 | S2 | i7 | TAAGAGTTGGCA | 469 | 0 |
| 1186 | S2-i7t0-TAAGACTCCATC | 470 | S2 | i7 | TAAGACTCCATC | 470 | 0 |
| 1187 | S2-i7t0-TGCCACATGTTT | 471 | S2 | i7 | TGCCACATGTTT | 471 | 0 |
| 1188 | S2-i7t0-AATGGACAAGCC | 472 | S2 | i7 | AATGGACAAGCC | 472 | 0 |
| 1189 | S2-i7t0-ATCCTCCAGGCA | 473 | S2 | i7 | ATCCTCCAGGCA | 473 | 0 |
| 1190 | S2-i7t0-GACTGAATAGAT | 474 | S2 | i7 | GACTGAATAGAT | 474 | 0 |
| 1191 | S2-i7t0-ATTCGGCTTTAA | 475 | S2 | i7 | ATTCGGCTTTAA | 475 | 0 |
| 1192 | S2-i7t0-GAAGTTTGTTAA | 476 | S2 | i7 | GAAGTTTGTTAA | 476 | 0 |
| 1193 | S2-i7t0-GAAGTCGAGGTA | 477 | S2 | i7 | GAAGTCGAGGTA | 477 | 0 |
| 1194 | S2-i7t0-GACCATTGTCGA | 478 | S2 | i7 | GACCATTGTCGA | 478 | 0 |
| 1195 | S2-i7t0-GCCCTCCAATCC | 479 | S2 | i7 | GCCCTCCAATCC | 479 | 0 |
| 1196 | S2-i7t0-GTCGTGTCGGGA | 480 | S2 | i7 | GTCGTGTCGGGA | 480 | 0 |
| 1197 | S2-i7t0-GCGTTTCGTACA | 481 | S2 | i7 | GCGTTTCGTACA | 481 | 0 |
| 1198 | S2-i7t0-TATAGTTGTCAT | 482 | S2 | i7 | TATAGTTGTCAT | 482 | 0 |
| 1199 | S2-i7t0-ATCCACGCGTGC | 483 | S2 | i7 | ATCCACGCGTGC | 483 | 0 |
| 1200 | S2-i7t0-CGATTTCTAGAG | 484 | S2 | i7 | CGATTTCTAGAG | 484 | 0 |
| 1201 | S2-i7t0-AACGCGACGTAA | 485 | S2 | i7 | AACGCGACGTAA | 485 | 0 |
| 1202 | S2-i7t0-GCTCAACCGACC | 486 | S2 | i7 | GCTCAACCGACC | 486 | 0 |
| 1203 | S2-i7t0-TACTGGTAACAT | 487 | S2 | i7 | TACTGGTAACAT | 487 | 0 |
| 1204 | S2-i7t0-ACATCAGAGAAT | 488 | S2 | i7 | ACATCAGAGAAT | 488 | 0 |
| 1205 | S2-i7t0-CTAGTAGAATTC | 489 | S2 | i7 | CTAGTAGAATTC | 489 | 0 |
| 1206 | S2-i7t0-AGCGCGGTACTG | 490 | S2 | i7 | AGCGCGGTACTG | 490 | 0 |
| 1207 | S2-i7t0-TTGAGATATGCA | 491 | S2 | i7 | TTGAGATATGCA | 491 | 0 |
| 1208 | S2-i7t0-TTACCCGTTTAA | 492 | S2 | i7 | TTACCCGTTTAA | 492 | 0 |
| 1209 | S2-i7t0-ACTAGTCTCATT | 493 | S2 | i7 | ACTAGTCTCATT | 493 | 0 |
| 1210 | S2-i7t0-TTGTTCTGTTGA | 494 | S2 | i7 | TTGTTCTGTTGA | 494 | 0 |
| 1211 | S2-i7t0-CAGTAAGGTGAG | 495 | S2 | i7 | CAGTAAGGTGAG | 495 | 0 |
| 1212 | S2-i7t0-AACGTTCGCCTG | 496 | S2 | i7 | AACGTTCGCCTG | 496 | 0 |
| 1213 | S2-i7t0-TCTGGTGCATTG | 497 | S2 | i7 | TCTGGTGCATTG | 497 | 0 |
| 1214 | S2-i7t0-TTCGGTGTCTAG | 498 | S2 | i7 | TTCGGTGTCTAG | 498 | 0 |
| 1215 | S2-i7t0-ATGTCTGAAATC | 499 | S2 | i7 | ATGTCTGAAATC | 499 | 0 |
| 1216 | S2-i7t0-TCTGGCCATGCT | 500 | S2 | i7 | TCTGGCCATGCT | 500 | 0 |
| 1217 | S2-i7t0-TAATGTCTTGAG | 501 | S2 | i7 | TAATGTCTTGAG | 501 | 0 |
| 1218 | S2-i7t0-CAGACTCACCCG | 502 | S2 | i7 | CAGACTCACCCG | 502 | 0 |
| 1219 | S2-i7t0-CTACTGATAGGA | 503 | S2 | i7 | CTACTGATAGGA | 503 | 0 |
| 1220 | S2-i7t0-ACGGGCTCGTTA | 504 | S2 | i7 | ACGGGCTCGTTA | 504 | 0 |
| 1221 | S2-i7t0-CATTGCAGACAC | 505 | S2 | i7 | CATTGCAGACAC | 505 | 0 |
| 1222 | S2-i7t0-GAATAAGGGACC | 506 | S2 | i7 | GAATAAGGGACC | 506 | 0 |
| 1223 | S2-i7t0-CATCAAGCCCTT | 507 | S2 | i7 | CATCAAGCCCTT | 507 | 0 |
| 1224 | S2-i7t0-TTTACCGAGCAA | 508 | S2 | i7 | TTTACCGAGCAA | 508 | 0 |
| 1225 | S2-i7t0-CTTTCCTGCGCG | 509 | S2 | i7 | CTTTCCTGCGCG | 509 | 0 |
| 1226 | S2-i7t0-CTTGTGCTATCT | 510 | S2 | i7 | CTTGTGCTATCT | 510 | 0 |
| 1227 | S2-i7t0-AGGAATCGCAAA | 511 | S2 | i7 | AGGAATCGCAAA | 511 | 0 |
| 1228 | S2-i7t0-TCTAGACACAGT | 512 | S2 | i7 | TCTAGACACAGT | 512 | 0 |
| 1229 | S2-i7t0-ACTCTCCATCCC | 513 | S2 | i7 | ACTCTCCATCCC | 513 | 0 |
| 1230 | S2-i7t0-ACGCCTAGGCGT | 514 | S2 | i7 | ACGCCTAGGCGT | 514 | 0 |
| 1231 | S2-i7t0-CAGCAACAACTG | 515 | S2 | i7 | CAGCAACAACTG | 515 | 0 |
| 1232 | S2-i7t0-CTTACATGAACC | 516 | S2 | i7 | CTTACATGAACC | 516 | 0 |
| 1233 | S2-i7t0-TCCTTTGTCGTG | 517 | S2 | i7 | TCCTTTGTCGTG | 517 | 0 |
| 1234 | S2-i7t0-AAGATCGTAAAC | 518 | S2 | i7 | AAGATCGTAAAC | 518 | 0 |
| 1235 | S2-i7t0-AGGAACATACTA | 519 | S2 | i7 | AGGAACATACTA | 519 | 0 |
| 1236 | S2-i7t0-GATGAATGATAC | 520 | S2 | i7 | GATGAATGATAC | 520 | 0 |
| 1237 | S2-i7t0-GAGAAATAATTC | 521 | S2 | i7 | GAGAAATAATTC | 521 | 0 |
| 1238 | S2-i7t0-GCCACTCGCATG | 522 | S2 | i7 | GCCACTCGCATG | 522 | 0 |
| 1239 | S2-i7t0-TGTAAATAGCCC | 523 | S2 | i7 | TGTAAATAGCCC | 523 | 0 |
| 1240 | S2-i7t0-CATAATTCGCTA | 524 | S2 | i7 | CATAATTCGCTA | 524 | 0 |
| 1241 | S2-i7t0-CATTACGCAGTT | 525 | S2 | i7 | CATTACGCAGTT | 525 | 0 |
| 1242 | S2-i7t0-GAGCGGATGATT | 526 | S2 | i7 | GAGCGGATGATT | 526 | 0 |
| 1243 | S2-i7t0-GTACATCGCCGC | 527 | S2 | i7 | GTACATCGCCGC | 527 | 0 |
| 1244 | S2-i7t0-AACACTCACTTG | 528 | S2 | i7 | AACACTCACTTG | 528 | 0 |
| 1245 | S2-i7t0-GTACCGGGCTAC | 529 | S2 | i7 | GTACCGGGCTAC | 529 | 0 |
| 1246 | S2-i7t0-TCGACCCATTAA | 530 | S2 | i7 | TCGACCCATTAA | 530 | 0 |
| 1247 | S2-i7t0-GCTTAACTACGC | 531 | S2 | i7 | GCTTAACTACGC | 531 | 0 |
| 1248 | S2-i7t0-TACTGCCCAACT | 532 | S2 | i7 | TACTGCCCAACT | 532 | 0 |
| 1249 | S2-i7t0-TGAGCTTGATCA | 533 | S2 | i7 | TGAGCTTGATCA | 533 | 0 |
| 1250 | S2-i7t0-GTCGCAGGACAT | 534 | S2 | i7 | GTCGCAGGACAT | 534 | 0 |
| 1251 | S2-i7t0-TAGCTCGGGTAA | 535 | S2 | i7 | TAGCTCGGGTAA | 535 | 0 |
| 1252 | S2-i7t0-CAACAGTTCCAC | 536 | S2 | i7 | CAACAGTTCCAC | 536 | 0 |
| 1253 | S2-i7t0-CGTCACCACTCA | 537 | S2 | i7 | CGTCACCACTCA | 537 | 0 |
| 1254 | S2-i7t0-GACACACTTGAT | 538 | S2 | i7 | GACACACTTGAT | 538 | 0 |
| 1255 | S2-i7t0-TGAGTTGCGTTC | 539 | S2 | i7 | TGAGTTGCGTTC | 539 | 0 |
| 1256 | S2-i7t0-CTACCCTAGTCG | 540 | S2 | i7 | CTACCCTAGTCG | 540 | 0 |
| 1257 | S2-i7t0-TAGATCTTGAGT | 541 | S2 | i7 | TAGATCTTGAGT | 541 | 0 |
| 1258 | S2-i7t0-TCTACCTCTAAC | 542 | S2 | i7 | TCTACCTCTAAC | 542 | 0 |
| 1259 | S2-i7t0-AGACGAAACGGA | 543 | S2 | i7 | AGACGAAACGGA | 543 | 0 |
| 1260 | S2-i7t0-CAAGGGCTTCCC | 544 | S2 | i7 | CAAGGGCTTCCC | 544 | 0 |
| 1261 | S2-i7t0-ACGCAATTGTAC | 545 | S2 | i7 | ACGCAATTGTAC | 545 | 0 |
| 1262 | S2-i7t0-AAATAAACTCGC | 546 | S2 | i7 | AAATAAACTCGC | 546 | 0 |
| 1263 | S2-i7t0-ACCATTAGCCGT | 547 | S2 | i7 | ACCATTAGCCGT | 547 | 0 |
| 1264 | S2-i7t0-ACCTATCCAGTG | 548 | S2 | i7 | ACCTATCCAGTG | 548 | 0 |
| 1265 | S2-i7t0-CGTGAATGGTGA | 549 | S2 | i7 | CGTGAATGGTGA | 549 | 0 |
| 1266 | S2-i7t0-TGCTACAAGGGA | 550 | S2 | i7 | TGCTACAAGGGA | 550 | 0 |
| 1267 | S2-i7t0-CTTCAGTACCGC | 551 | S2 | i7 | CTTCAGTACCGC | 551 | 0 |
| 1268 | S2-i7t0-CAAACCGCTCTA | 552 | S2 | i7 | CAAACCGCTCTA | 552 | 0 |
| 1269 | S2-i7t0-ATGCCGGTCCGC | 553 | S2 | i7 | ATGCCGGTCCGC | 553 | 0 |
| 1270 | S2-i7t0-GCTGTCGTGGGA | 554 | S2 | i7 | GCTGTCGTGGGA | 554 | 0 |
| 1271 | S2-i7t0-TCCCGCCTCCGA | 555 | S2 | i7 | TCCCGCCTCCGA | 555 | 0 |
| 1272 | S2-i7t0-GAACCTAATTTG | 556 | S2 | i7 | GAACCTAATTTG | 556 | 0 |
| 1273 | S2-i7t0-TCCAGTATCGAC | 557 | S2 | i7 | TCCAGTATCGAC | 557 | 0 |
| 1274 | S2-i7t0-TCACGGTCAGCA | 558 | S2 | i7 | TCACGGTCAGCA | 558 | 0 |
| 1275 | S2-i7t0-TGTTGCTCTCTG | 559 | S2 | i7 | TGTTGCTCTCTG | 559 | 0 |
| 1276 | S2-i7t0-GTTTGCGCAAAT | 560 | S2 | i7 | GTTTGCGCAAAT | 560 | 0 |
| 1277 | S2-i7t0-CAAAGCACCGTG | 561 | S2 | i7 | CAAAGCACCGTG | 561 | 0 |
| 1278 | S2-i7t0-CTGAGGCATGGC | 562 | S2 | i7 | CTGAGGCATGGC | 562 | 0 |
| 1279 | S2-i7t0-CAAGGATCTCTT | 563 | S2 | i7 | CAAGGATCTCTT | 563 | 0 |
| 1280 | S2-i7t0-TCAAGGCCTGTT | 564 | S2 | i7 | TCAAGGCCTGTT | 564 | 0 |
| 1281 | S2-i7t0-AACCTGTCCGTA | 565 | S2 | i7 | AACCTGTCCGTA | 565 | 0 |
| 1282 | S2-i7t0-TAACCACGAAGT | 566 | S2 | i7 | TAACCACGAAGT | 566 | 0 |
| 1283 | S2-i7t0-TTCTCCTAGGAA | 567 | S2 | i7 | TTCTCCTAGGAA | 567 | 0 |
| 1284 | S2-i7t0-AAATTGTACTTC | 568 | S2 | i7 | AAATTGTACTTC | 568 | 0 |
| 1285 | S2-i7t0-CTCCACCGAGTT | 569 | S2 | i7 | CTCCACCGAGTT | 569 | 0 |
| 1286 | S2-i7t0-CAGTTGTATTCG | 570 | S2 | i7 | CAGTTGTATTCG | 570 | 0 |
| 1287 | S2-i7t0-TTGGGTAGCAGC | 571 | S2 | i7 | TTGGGTAGCAGC | 571 | 0 |
| 1288 | S2-i7t0-TATATCGCTTGC | 572 | S2 | i7 | TATATCGCTTGC | 572 | 0 |
| 1289 | S2-i7t0-GACTCGTTGACC | 573 | S2 | i7 | GACTCGTTGACC | 573 | 0 |
| 1290 | S2-i7t0-TAACTCTGGCCC | 574 | S2 | i7 | TAACTCTGGCCC | 574 | 0 |
| 1291 | S2-i7t0-GCACTAGGTTTG | 575 | S2 | i7 | GCACTAGGTTTG | 575 | 0 |
| 1292 | S2-i7t0-CATGCTTCCGTT | 576 | S2 | i7 | CATGCTTCCGTT | 576 | 0 |
| 1293 | S2-i7t0-CACCTGTGATGA | 577 | S2 | i7 | CACCTGTGATGA | 577 | 0 |
| 1294 | S2-i7t0-ACCTATTTAAGA | 578 | S2 | i7 | ACCTATTTAAGA | 578 | 0 |
| 1295 | S2-i7t0-GCACGTAGGTAA | 579 | S2 | i7 | GCACGTAGGTAA | 579 | 0 |
| 1296 | S2-i7t0-CAGTACTGTTCC | 580 | S2 | i7 | CAGTACTGTTCC | 580 | 0 |
| 1297 | S2-i7t0-GTTAGGAACCTA | 581 | S2 | i7 | GTTAGGAACCTA | 581 | 0 |
| 1298 | S2-i7t0-TAGATTGCTGAT | 582 | S2 | i7 | TAGATTGCTGAT | 582 | 0 |
| 1299 | S2-i7t0-ATGAGTCATAGT | 583 | S2 | i7 | ATGAGTCATAGT | 583 | 0 |
| 1300 | S2-i7t0-AGAAATGATCCG | 584 | S2 | i7 | AGAAATGATCCG | 584 | 0 |
| 1301 | S2-i7t0-GATATTCATCCG | 585 | S2 | i7 | GATATTCATCCG | 585 | 0 |
| 1302 | S2-i7t0-TCGGTTCCGTCC | 586 | S2 | i7 | TCGGTTCCGTCC | 586 | 0 |
| 1303 | S2-i7t0-AGACACAGGTAG | 587 | S2 | i7 | AGACACAGGTAG | 587 | 0 |
| 1304 | S2-i7t0-AGGTTGGGCCGC | 588 | S2 | i7 | AGGTTGGGCCGC | 588 | 0 |
| 1305 | S2-i7t0-GTAGAACCTCTG | 589 | S2 | i7 | GTAGAACCTCTG | 589 | 0 |
| 1306 | S2-i7t0-AACTATGCGAAA | 590 | S2 | i7 | AACTATGCGAAA | 590 | 0 |
| 1307 | S2-i7t0-TGCGACGCCACG | 591 | S2 | i7 | TGCGACGCCACG | 591 | 0 |
| 1308 | S2-i7t0-CGTAAGTCGCGT | 592 | S2 | i7 | CGTAAGTCGCGT | 592 | 0 |
| 1309 | S2-i7t0-CTGAGACTTGTT | 593 | S2 | i7 | CTGAGACTTGTT | 593 | 0 |
| 1310 | S2-i7t0-AGTCAGTAAGAG | 594 | S2 | i7 | AGTCAGTAAGAG | 594 | 0 |
| 1311 | S2-i7t0-CAGCGAAGCGAA | 595 | S2 | i7 | CAGCGAAGCGAA | 595 | 0 |
| 1312 | S2-i7t0-AATGTTGTGCGA | 596 | S2 | i7 | AATGTTGTGCGA | 596 | 0 |
| 1313 | S2-i7t0-TTCCATATCACC | 597 | S2 | i7 | TTCCATATCACC | 597 | 0 |
| 1314 | S2-i7t0-GTCTCAAATTGT | 598 | S2 | i7 | GTCTCAAATTGT | 598 | 0 |
| 1315 | S2-i7t0-GCGGGATGTTTA | 599 | S2 | i7 | GCGGGATGTTTA | 599 | 0 |
| 1316 | S2-i7t0-GATACTAAGGAC | 600 | S2 | i7 | GATACTAAGGAC | 600 | 0 |
| 1317 | S2-i7t0-CGAAACATTAAT | 601 | S2 | i7 | CGAAACATTAAT | 601 | 0 |
| 1318 | S2-i7t0-TACGATAGGTCA | 602 | S2 | i7 | TACGATAGGTCA | 602 | 0 |
| 1319 | S2-i7t0-GCACAATGCACG | 603 | S2 | i7 | GCACAATGCACG | 603 | 0 |
| 1320 | S2-i7t0-GTCCACCGTCAA | 604 | S2 | i7 | GTCCACCGTCAA | 604 | 0 |
| 1321 | S2-i7t0-TGAATCCAACGA | 605 | S2 | i7 | TGAATCCAACGA | 605 | 0 |
| 1322 | S2-i7t0-GACAATCGTCAG | 606 | S2 | i7 | GACAATCGTCAG | 606 | 0 |
| 1323 | S2-i7t0-TCGCTTGATATT | 607 | S2 | i7 | TCGCTTGATATT | 607 | 0 |
| 1324 | S2-i7t0-ACACACACCGCT | 608 | S2 | i7 | ACACACACCGCT | 608 | 0 |
| 1325 | S2-i7t0-TGACCACCAATA | 609 | S2 | i7 | TGACCACCAATA | 609 | 0 |
| 1326 | S2-i7t0-CGCCTGGGTAAG | 610 | S2 | i7 | CGCCTGGGTAAG | 610 | 0 |
| 1327 | S2-i7t0-TCGGTATCAGTG | 611 | S2 | i7 | TCGGTATCAGTG | 611 | 0 |
| 1328 | S2-i7t0-GAGTCCTGATAT | 612 | S2 | i7 | GAGTCCTGATAT | 612 | 0 |
| 1329 | S2-i7t0-CACTACACATGT | 613 | S2 | i7 | CACTACACATGT | 613 | 0 |
| 1330 | S2-i7t0-CAGTAATTCCAT | 614 | S2 | i7 | CAGTAATTCCAT | 614 | 0 |
| 1331 | S2-i7t0-AATTAAACACCG | 615 | S2 | i7 | AATTAAACACCG | 615 | 0 |
| 1332 | S2-i7t0-GCAGGGCTTGCA | 616 | S2 | i7 | GCAGGGCTTGCA | 616 | 0 |
| 1333 | S2-i7t0-ACCCAAGCCATT | 617 | S2 | i7 | ACCCAAGCCATT | 617 | 0 |
| 1334 | S2-i7t0-AATTATACTGTG | 618 | S2 | i7 | AATTATACTGTG | 618 | 0 |
| 1335 | S2-i7t0-GAGCAGCGAAGT | 619 | S2 | i7 | GAGCAGCGAAGT | 619 | 0 |
| 1336 | S2-i7t0-CGCCGTACCGTA | 620 | S2 | i7 | CGCCGTACCGTA | 620 | 0 |
| 1337 | S2-i7t0-TTAGCTCCGGTT | 621 | S2 | i7 | TTAGCTCCGGTT | 621 | 0 |
| 1338 | S2-i7t0-GATACGTAGATC | 622 | S2 | i7 | GATACGTAGATC | 622 | 0 |
| 1339 | S2-i7t0-ACTTCACTCCTA | 623 | S2 | i7 | ACTTCACTCCTA | 623 | 0 |
| 1340 | S2-i7t0-AGGTTCATTAGA | 624 | S2 | i7 | AGGTTCATTAGA | 624 | 0 |
| 1341 | S2-i7t0-TGGTCATATAAT | 625 | S2 | i7 | TGGTCATATAAT | 625 | 0 |
| 1342 | S2-i7t0-GAAAGGCGTACT | 626 | S2 | i7 | GAAAGGCGTACT | 626 | 0 |
| 1343 | S2-i7t0-AACGCCTGGAAG | 627 | S2 | i7 | AACGCCTGGAAG | 627 | 0 |
| 1344 | S2-i7t0-AAGGTAGTGGCA | 628 | S2 | i7 | AAGGTAGTGGCA | 628 | 0 |
| 1345 | S2-i7t0-TGGCCAATCCAG | 629 | S2 | i7 | TGGCCAATCCAG | 629 | 0 |
| 1346 | S2-i7t0-TCGACAGCGATT | 630 | S2 | i7 | TCGACAGCGATT | 630 | 0 |
| 1347 | S2-i7t0-TTAACCACCGTT | 631 | S2 | i7 | TTAACCACCGTT | 631 | 0 |
| 1348 | S2-i7t0-TAGAGTTCTATC | 632 | S2 | i7 | TAGAGTTCTATC | 632 | 0 |
| 1349 | S2-i7t0-ACCAGATTGGAG | 633 | S2 | i7 | ACCAGATTGGAG | 633 | 0 |
| 1350 | S2-i7t0-ATCCTTACTTTA | 634 | S2 | i7 | ATCCTTACTTTA | 634 | 0 |
| 1351 | S2-i7t0-GCTACGCGGTGC | 635 | S2 | i7 | GCTACGCGGTGC | 635 | 0 |
| 1352 | S2-i7t0-TACTTCTTTGAA | 636 | S2 | i7 | TACTTCTTTGAA | 636 | 0 |
| 1353 | S2-i7t0-ACAAGAGATGCT | 637 | S2 | i7 | ACAAGAGATGCT | 637 | 0 |
| 1354 | S2-i7t0-ACCCAGGATGCC | 638 | S2 | i7 | ACCCAGGATGCC | 638 | 0 |
| 1355 | S2-i7t0-GTAGTTGGAACA | 639 | S2 | i7 | GTAGTTGGAACA | 639 | 0 |
| 1356 | S2-i7t0-TCTACATTTCTG | 640 | S2 | i7 | TCTACATTTCTG | 640 | 0 |
| 1357 | S2-i7t0-GTGCGCCGCTTC | 641 | S2 | i7 | GTGCGCCGCTTC | 641 | 0 |
| 1358 | S2-i7t0-GAACATCAGAAT | 642 | S2 | i7 | GAACATCAGAAT | 642 | 0 |
| 1359 | S2-i7t0-GCCGAAGCTGTC | 643 | S2 | i7 | GCCGAAGCTGTC | 643 | 0 |
| 1360 | S2-i7t0-AAGGACCCGAAT | 644 | S2 | i7 | AAGGACCCGAAT | 644 | 0 |
| 1361 | S2-i7t0-CTTTGCCGTTAT | 645 | S2 | i7 | CTTTGCCGTTAT | 645 | 0 |
| 1362 | S2-i7t0-AATCTCTGCCAT | 646 | S2 | i7 | AATCTCTGCCAT | 646 | 0 |
| 1363 | S2-i7t0-CGAGATTACCAG | 647 | S2 | i7 | CGAGATTACCAG | 647 | 0 |
| 1364 | S2-i7t0-GCTCGCAGCTAC | 648 | S2 | i7 | GCTCGCAGCTAC | 648 | 0 |
| 1365 | S2-i7t0-ACTCAGCCCGTT | 649 | S2 | i7 | ACTCAGCCCGTT | 649 | 0 |
| 1366 | S2-i7t0-TGATCGCATGCA | 650 | S2 | i7 | TGATCGCATGCA | 650 | 0 |
| 1367 | S2-i7t0-TTCAACAGATAC | 651 | S2 | i7 | TTCAACAGATAC | 651 | 0 |
| 1368 | S2-i7t0-GCTGATAGAAGT | 652 | S2 | i7 | GCTGATAGAAGT | 652 | 0 |
| 1369 | S2-i7t0-GTAGAATAGAAT | 653 | S2 | i7 | GTAGAATAGAAT | 653 | 0 |
| 1370 | S2-i7t0-CTCGAGCGATAA | 654 | S2 | i7 | CTCGAGCGATAA | 654 | 0 |
| 1371 | S2-i7t0-TGGTAGCGGCCA | 655 | S2 | i7 | TGGTAGCGGCCA | 655 | 0 |
| 1372 | S2-i7t0-AAACGGTCTTGA | 656 | S2 | i7 | AAACGGTCTTGA | 656 | 0 |
| 1373 | S2-i7t0-CAGCGCGTCCCT | 657 | S2 | i7 | CAGCGCGTCCCT | 657 | 0 |
| 1374 | S2-i7t0-TCTCCGATATAA | 658 | S2 | i7 | TCTCCGATATAA | 658 | 0 |
| 1375 | S2-i7t0-GACATCGCCTTT | 659 | S2 | i7 | GACATCGCCTTT | 659 | 0 |
| 1376 | S2-i7t0-TACCTAGATTCG | 660 | S2 | i7 | TACCTAGATTCG | 660 | 0 |
| 1377 | S2-i7t0-TGCAAGCTCTCG | 661 | S2 | i7 | TGCAAGCTCTCG | 661 | 0 |
| 1378 | S2-i7t0-CATTAGCCATAG | 662 | S2 | i7 | CATTAGCCATAG | 662 | 0 |
| 1379 | S2-i7t0-TGTCCTCAAGCA | 663 | S2 | i7 | TGTCCTCAAGCA | 663 | 0 |
| 1380 | S2-i7t0-TGGCTGCAAATA | 664 | S2 | i7 | TGGCTGCAAATA | 664 | 0 |
| 1381 | S2-i7t0-AACCTCATGCAG | 665 | S2 | i7 | AACCTCATGCAG | 665 | 0 |
| 1382 | S2-i7t0-AGAGCTCGAACG | 666 | S2 | i7 | AGAGCTCGAACG | 666 | 0 |
| 1383 | S2-i7t0-ATTCTTCGTGCT | 667 | S2 | i7 | ATTCTTCGTGCT | 667 | 0 |
| 1384 | S2-i7t0-GAACCAAGCCTA | 668 | S2 | i7 | GAACCAAGCCTA | 668 | 0 |
| 1385 | S2-i7t0-AGTTACTACAAG | 669 | S2 | i7 | AGTTACTACAAG | 669 | 0 |
| 1386 | S2-i7t0-TTGATCCCGAAT | 670 | S2 | i7 | TTGATCCCGAAT | 670 | 0 |
| 1387 | S2-i7t0-CTTGGCACCAGT | 671 | S2 | i7 | CTTGGCACCAGT | 671 | 0 |
| 1388 | S2-i7t0-GAAGAGGTAACC | 672 | S2 | i7 | GAAGAGGTAACC | 672 | 0 |
| 1389 | S2-i7t0-ACGACCGGGTTG | 673 | S2 | i7 | ACGACCGGGTTG | 673 | 0 |
| 1390 | S2-i7t0-GCATTTAGCTCA | 674 | S2 | i7 | GCATTTAGCTCA | 674 | 0 |
| 1391 | S2-i7t0-TGTCGTTGCCGA | 675 | S2 | i7 | TGTCGTTGCCGA | 675 | 0 |
| 1392 | S2-i7t0-AGAAAGCTTCTG | 676 | S2 | i7 | AGAAAGCTTCTG | 676 | 0 |
| 1393 | S2-i7t0-TAGTGGTCTGCA | 677 | S2 | i7 | TAGTGGTCTGCA | 677 | 0 |
| 1394 | S2-i7t0-GTCGAGGTCTAC | 678 | S2 | i7 | GTCGAGGTCTAC | 678 | 0 |
| 1395 | S2-i7t0-TCTTCAGCGACC | 679 | S2 | i7 | TCTTCAGCGACC | 679 | 0 |
| 1396 | S2-i7t0-TCAGCTAACTCA | 680 | S2 | i7 | TCAGCTAACTCA | 680 | 0 |
| 1397 | S2-i7t0-TCCAATCGACCT | 681 | S2 | i7 | TCCAATCGACCT | 681 | 0 |
| 1398 | S2-i7t0-CGGTCTGATATA | 682 | S2 | i7 | CGGTCTGATATA | 682 | 0 |
| 1399 | S2-i7t0-GAGGGCAATTCA | 683 | S2 | i7 | GAGGGCAATTCA | 683 | 0 |
| 1400 | S2-i7t0-ACGGTCTTCGAG | 684 | S2 | i7 | ACGGTCTTCGAG | 684 | 0 |
| 1401 | S2-i7t0-ACCGAGTTGTCT | 685 | S2 | i7 | ACCGAGTTGTCT | 685 | 0 |
| 1402 | S2-i7t0-TGCGAGCGTCCC | 686 | S2 | i7 | TGCGAGCGTCCC | 686 | 0 |
| 1403 | S2-i7t0-CTTGCACATACT | 687 | S2 | i7 | CTTGCACATACT | 687 | 0 |
| 1404 | S2-i7t0-ACCTTAGTGTCC | 688 | S2 | i7 | ACCTTAGTGTCC | 688 | 0 |
| 1405 | S2-i7t0-CTGTAGTTCTCG | 689 | S2 | i7 | CTGTAGTTCTCG | 689 | 0 |
| 1406 | S2-i7t0-AGGCCCTTAGTC | 690 | S2 | i7 | AGGCCCTTAGTC | 690 | 0 |
| 1407 | S2-i7t0-CAACGATACTAA | 691 | S2 | i7 | CAACGATACTAA | 691 | 0 |
| 1408 | S2-i7t0-GCCAAATGCGAA | 692 | S2 | i7 | GCCAAATGCGAA | 692 | 0 |
| 1409 | S2-i7t0-TCCAAAGTTCTC | 693 | S2 | i7 | TCCAAAGTTCTC | 693 | 0 |
| 1410 | S2-i7t0-ACCCGGACTATC | 694 | S2 | i7 | ACCCGGACTATC | 694 | 0 |
| 1411 | S2-i7t0-CAACAATGTGAA | 695 | S2 | i7 | CAACAATGTGAA | 695 | 0 |
| 1412 | S2-i7t0-GTCATCATTACA | 696 | S2 | i7 | GTCATCATTACA | 696 | 0 |
| 1413 | S2-i7t0-CGTATTTGAACT | 697 | S2 | i7 | CGTATTTGAACT | 697 | 0 |
| 1414 | S2-i7t0-CACACATGTGCT | 698 | S2 | i7 | CACACATGTGCT | 698 | 0 |
| 1415 | S2-i7t0-AGGTCTCGATAG | 699 | S2 | i7 | AGGTCTCGATAG | 699 | 0 |
| 1416 | S2-i7t0-CGAAATTGAAAC | 700 | S2 | i7 | CGAAATTGAAAC | 700 | 0 |
| 1417 | S2-i7t0-TCTGGCAAACAA | 701 | S2 | i7 | TCTGGCAAACAA | 701 | 0 |
| 1418 | S2-i7t0-CGGATTGAACGA | 702 | S2 | i7 | CGGATTGAACGA | 702 | 0 |
| 1419 | S2-i7t0-TATCAAGTGATC | 703 | S2 | i7 | TATCAAGTGATC | 703 | 0 |
| 1420 | S2-i7t0-ACAGTAAGTGTA | 704 | S2 | i7 | ACAGTAAGTGTA | 704 | 0 |
| 1421 | S2-i7t0-ATGCGCGCAGCA | 705 | S2 | i7 | ATGCGCGCAGCA | 705 | 0 |
| 1422 | S2-i7t0-AGCGGACGTGCA | 706 | S2 | i7 | AGCGGACGTGCA | 706 | 0 |
| 1423 | S2-i7t0-TAACGTATGAAC | 707 | S2 | i7 | TAACGTATGAAC | 707 | 0 |
| 1424 | S2-i7t0-ATTGCCCTCATG | 708 | S2 | i7 | ATTGCCCTCATG | 708 | 0 |
| 1425 | S2-i7t0-CTTCCAACATAA | 709 | S2 | i7 | CTTCCAACATAA | 709 | 0 |
| 1426 | S2-i7t0-CGCCCTTACCCA | 710 | S2 | i7 | CGCCCTTACCCA | 710 | 0 |
| 1427 | S2-i7t0-CAACAATCGATT | 711 | S2 | i7 | CAACAATCGATT | 711 | 0 |
| 1428 | S2-i7t0-CAGGTTCGCCGT | 712 | S2 | i7 | CAGGTTCGCCGT | 712 | 0 |
| 1429 | S2-i7t0-TGATGCAATAGT | 713 | S2 | i7 | TGATGCAATAGT | 713 | 0 |
| 1430 | S2-i7t0-AATCCGCTCCGT | 714 | S2 | i7 | AATCCGCTCCGT | 714 | 0 |
| 1431 | S2-i7t0-CTCGGCGTTCAT | 715 | S2 | i7 | CTCGGCGTTCAT | 715 | 0 |
| 1432 | S2-i7t0-TGTTCAGGTCCC | 716 | S2 | i7 | TGTTCAGGTCCC | 716 | 0 |
| 1433 | S2-i7t0-CATCCTTAAGTC | 717 | S2 | i7 | CATCCTTAAGTC | 717 | 0 |
| 1434 | S2-i7t0-AGGACTGTTGCT | 718 | S2 | i7 | AGGACTGTTGCT | 718 | 0 |
| 1435 | S2-i7t0-TTGCGAACTCTA | 719 | S2 | i7 | TTGCGAACTCTA | 719 | 0 |
| 1436 | S2-i7t0-GTTCTGGATTTA | 720 | S2 | i7 | GTTCTGGATTTA | 720 | 0 |
| 1437 | S2-i7t0-TTCTGGTCCACC | 721 | S2 | i7 | TTCTGGTCCACC | 721 | 0 |
| 1438 | S2-i7t0-CACACCAAATCA | 722 | S2 | i7 | CACACCAAATCA | 722 | 0 |
| 1439 | S2-i7t0-AGCGAACTGGGC | 723 | S2 | i7 | AGCGAACTGGGC | 723 | 0 |
| 1440 | S2-i7t0-GAGCTCGGGCTT | 724 | S2 | i7 | GAGCTCGGGCTT | 724 | 0 |
| 1441 | S2-i7t0-GCTCCTGTCGGT | 725 | S2 | i7 | GCTCCTGTCGGT | 725 | 0 |
| 1442 | S2-i7t0-TGCTTCCCTCAA | 726 | S2 | i7 | TGCTTCCCTCAA | 726 | 0 |
| 1443 | S2-i7t0-TGGAGATAATTG | 727 | S2 | i7 | TGGAGATAATTG | 727 | 0 |
| 1444 | S2-i7t0-AATATTCAGGCT | 728 | S2 | i7 | AATATTCAGGCT | 728 | 0 |
| 1445 | S2-i7t0-TGTATGAATGCC | 729 | S2 | i7 | TGTATGAATGCC | 729 | 0 |
| 1446 | S2-i7t0-ATTGAGACCCAC | 730 | S2 | i7 | ATTGAGACCCAC | 730 | 0 |
| 1447 | S2-i7t0-CAGGATCCTATC | 731 | S2 | i7 | CAGGATCCTATC | 731 | 0 |
| 1448 | S2-i7t0-GTGGCTCGTTCT | 732 | S2 | i7 | GTGGCTCGTTCT | 732 | 0 |
| 1449 | S2-i7t0-CGTTTCGAGGCG | 733 | S2 | i7 | CGTTTCGAGGCG | 733 | 0 |

In some embodiments, a primer includes (a) a block of 12 nucleotides corresponding to the appropriate sequencing barcode, and (b) a 0-3 nucleotides diversity spacer, where (a) and (b) are 5′ to (c) the targeting sequence that increase the base diversity at each sequencing position to improve the quality of base calling; each barcode is paired with a specific spacer length.

In some embodiments, “unified” primers are used. These primers have all of the components required for every step of amplifying the target and performing in an Illumina flowcell. Previous amplicon designs are highly compact, using custom sequencing primers to read the i5 (on NextSeq; NovaSeq chemistry does not use this), i7 and diagnostic sequence. They can be schematized to comprise three parts: (a) an Illumina flowcell binding sequence; (b) a sequencing index; and (c) a specific region that is used for targeting and binding all of the necessary sequencing primers. The previous design has the advantage of less expensive synthesis, but because some amplified sequence is used for binding of the sequencing primers it cannot sequence any PCR artifacts, which it is believed leads to a number of performance problems on Illumina sequencers.

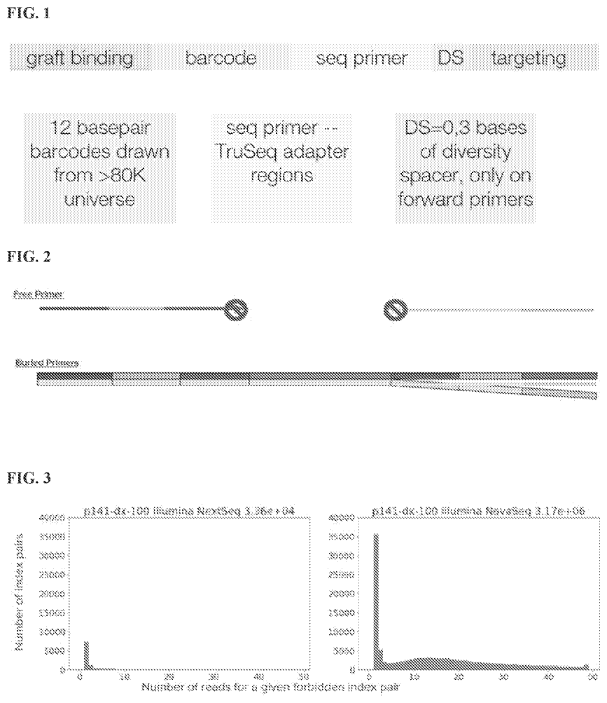

In some embodiments, the primers have been redesigned to typical Illumina schemes, though such unified primers are not in common use. Referring to FIG. 1, the designs disclosed comprise of (a) an Illumina flowcell binding sequence (a “graft binding” sequence); (b) a sequencing index (“barcode”); (c) an Illumina standard region which is used for binding of all sequencing primers (“seq primer”); (d) a diversity spacer (“DS”) of 0 to 3 bases specific to an index; and (e) the targeting sequence. This results in much longer primers than previous designs, but they can sequence all PCR artifacts, enabling these to be measured for selection of indexes as well as putting sequencer output in better correlation with diagnostic measures such as qPCR quantification of the input libraries. The disclosed design also obviates the need to add custom primers to the sequencing cartridge, streamlining the standard operation procedures and eliminating a point-of-failure.

In some embodiments, the index sequence of a primer is 10 or more base pairs (e.g., 12 base pairs) that allow certain computational properties such that they cannot be confused with each other without a defined number of errors, and they lack long runs of the same nucleotide (“homopolymers”).

### Processing of Indexed Sequencing Template

### Index Hopping

In some embodiments of the methods provided herein, samples comprising indexed sequencing templates (e.g., indexed amplification products) are subjected to a clean-up treatment and/or a processing step prior to sequencing. For example, in certain embodiments it is desirable to reduce the concentration of free and/or buried index primers present in the library of indexed sequencing templates prior to sequencing. When certain sequencing processes are used, such primers can increase index hopping and decrease data quality. The term “free primers” refers to unextended primers remaining free in the sample following completion of the amplification reaction used to produce indexed sequencing templates (illustrated in FIG. 2). The term “buried primers” refers to unextended primers that are annealed or otherwise associated with an indexed sequencing template present in the sample following completion of the amplification reaction used to produce indexed sequencing templates (illustrated in FIG. 2). Notably, buried primers can be resistant to sequencing template purification methods.

Index hopping refers to extension products that comprise one or more improper index sequences resulting from the presence of free or buried primers. One way to determine the prevalence of index hopping is to look at how many reads contain forbidden index pairs. For example, in an indexing process that includes 1536 index pairs (i.e., 1536 forward indices, each paired with a specific reverse index), there would be 1536 valid index pairs (i.e., having a forward index matched with the correct reverse index) and 2,357,760 forbidden index pairs (i.e., an incorrect pairing of a forward index and a reverse index. If index hopping did not exist, these no sequencing reads would include forbidden index pairs. Moreover, the greater the frequency of index hopping, the greater the percentage of reads that will have forbidden index pairs.

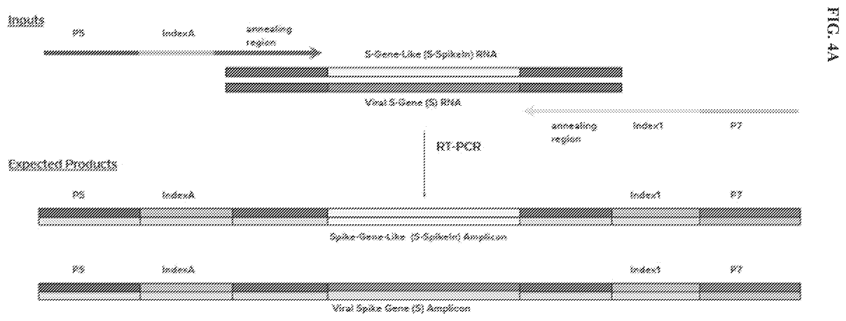

Referring to the left histogram of FIG. 3, the number of index hopping events for each barcode on an Illumina NextSeq platform shows that index hopping on this platform is rare. Specifically, the graph shows that about 7000 forbidden index pairs appear in 1 read, about 1000 forbidden index pairs appear in 2 reads, and a few hundred forbidden index pairs appear in more than 2 reads (up to about 10 for some forbidden index pairs). This illustrates that the vast majority of the 2,357,766 possible forbidden index pairs do not appear in any sequencing reads (these are not shown).

The right histogram of FIG. 3 is from a NovaSeq platform assay. This platform is characterized by a pronounced higher frequency of index hopping. For example, about 35,000 forbidden index pairs appear in one read, about 5000 forbidden index pairs appear in 2 reads, and a many thousands of pairs of forbidden index reads appear more than 2 reads (up to about 50 reads for some forbidden index pairs). In total, there are about 3,170,600 index hopped reads containing forbidden index pairs.

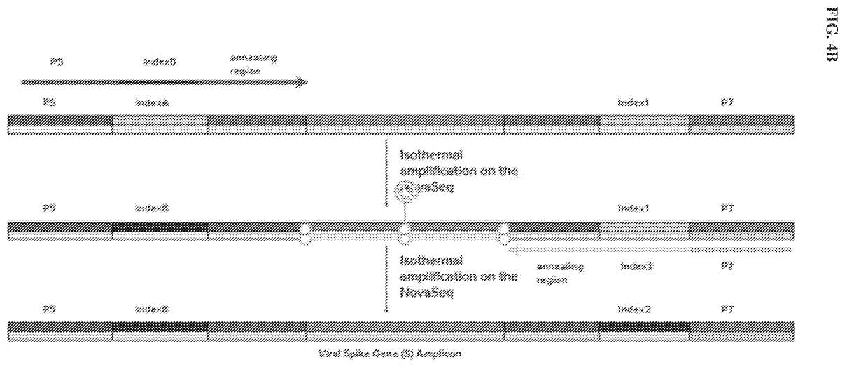

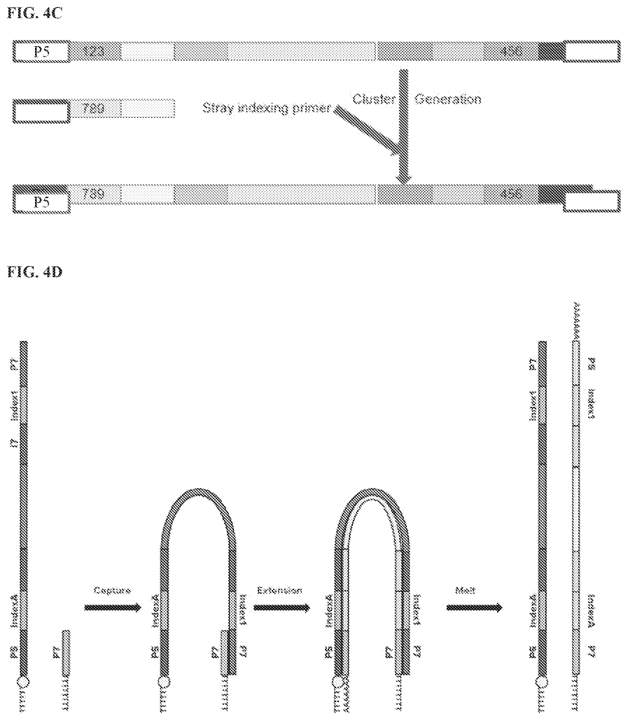

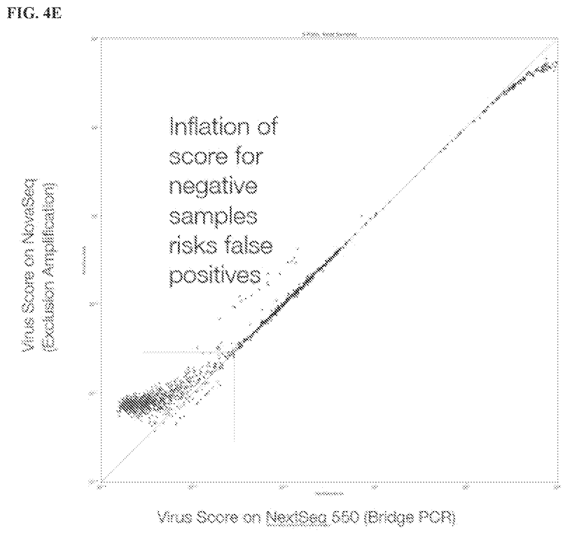

In certain aspects of the assays provided herein, a subject sample is mixed with a unique (indexed) forward primer and a unique (indexed) reverse primer. Thus, in the absence of index hopping, amplification products having the “A1” indexed primer set corresponds to subject A1 (FIG. 4A). In all samples, the forward and reverse primers can amplify the target nucleic acid molecule, resulting in a doubly-indexed amplicon. The doubly-indexed amplicons from many subjects (A1, B2, C3, D4 . . . ) are mixed and sequenced together.

However, during the amplification step of a sequencing modality prone to index hopping, such as the NovaSeq sequencing process, there is opportunity for an indexed primer from subject B2 to extend, using the S Amplicon from subject A1 as the template, making a “B1” chimera (FIG. 4B; see also FIG. 4C). This B1 chimera has the subject B2 index on one side, but the subject A1 index on the other side. This “single hop” product should not exist and can be safely ignored/filtered out as a “forbidden index pair.”

In some cases, however, another primer from subject B2 can extend in the other direction using the B1 chimera as a template. The “double hop” product now has the index from subject B2 on both sides, resulting in a false read indistinguishable from true reads for subject B2 (FIG. 4B).

The NextSeq platform uses a bridge amplification technique to generate amplicons for sequencing (FIG. 4D). In this amplification scheme, the initial extension product serves as the template for a second extension. This iterative cycle continues until there are many copies of the amplicon clustered together. In general, the exclusion amplification chemistry used in the NovaSeq platform is isothermal, which provides more opportunity for free primers to accumulate and promote index hopping relative to the NextSeq platform, which utilizes PCR (thermocycling). This increased index hopping frequency can lead to dual hopping events, thereby generating false but apparently “valid” reads. As shown in FIG. 4E, increased false positives are observed in the NovaSeq platform compared to the NextSeq platform.\

As provided herein, the frequency of index hopping can be reduced when performing index hopping-prone sequencing platforms, such as the NovaSeq platform, by reducing the concentration of free and/or buried primers in the indexed amplification product prior to initiating the sequencing process. Thus, in certain embodiments, strategies are provided herein for reducing and/or eliminating index hopping centered around removing or neutralizing free and/or buried primers so that they cannot extend or are extended to include an irrelevant sequence, thereby reducing their ability to participate in index hopping during sequencing (for example, on the NovaSeq platform). In certain embodiments, a combination of the index hopping reduction methods are performed prior to sequencing (i.e., a combination of 2, 3, 4, or more of the index processing methods provided herein are performed).

Primers residing inside or otherwise associated with larger complexes are less susceptible to inactivation using methods to inactivate free primers due to being “buried.” Thus, the methods described herein can comprise assays to remove or inactivate free primers, buried primers, or both. Thus, the methods can be combinations of more than one strategy for eliminating contaminating primers.

### High Performance Liquid Chromatography

In some embodiments, a sample comprising an indexed sequencing template is purified using a High Performance Liquid Chromatography (HPLC) process prior to sequencing in order to reduce the concentration of free primers in the sample. For example, in some embodiments, the library of indexed sequencing templates is purified on an HPLC column such as HPLC purification of DNA oligonucleotides using Ion Exchange or Ion-Pairing Reverse Phase (IP-RP) chromatography. This technique separates DNA oligonucleotides based on size and allows isolation of longer PCR products from contaminating primers of shorter length. In some embodiments, prior to HPLC purification, a sample comprising indexed sequencing templates is treated by any process or reagent described herein to free buried primers.

In some embodiments, the sample is further treated with FAB (Free Adapter Blocking) reagent (Illumina, San Diego, CA) before and/or after HPLC purification.

After purification (mostly) single-stranded amplicons remain. However, HPLC alone may not be sufficient to remove all free or buried primers. Thus, in some circumstances, index hopping occurs when only HPLC is used as a treatment. In some embodiments, HPLC purification can precede an enzymatic treatment to remove those primers not removed during the HPLC purification. In some embodiments, the enzymatic treatment includes the FAB reagent. Thus, in some embodiments, FAB reagent and/or HPLC fractionation are used to block excess free adapter, remove free index primers from the library, and to reduce index hopping and enhance data quality.

In certain cases, buried primers may be present that are resistant to size-based separation techniques due to their binding to longer amplicons. Thus, in certain embodiments, HPLC purification is performed under denaturing conditions. Denaturing conditions for use in HPLC purification processes can be generated by adjusting the pH of the sample (e.g., to a pH of at least about 12) and/or by adjusting the temperature of the sample (e.g., to a temperature of at least about 85° C.).

In some embodiments, the patient sample is treated with FAB reagent prior to sequencing. In some embodiments, the patient sample is treated with FAB reagent prior to sequencing (e.g., running on NovaSeq). In some embodiments, the patient sample is purified via HPLC prior to sequencing (e.g., running on NovaSeq).

In some embodiments, clean-up treatment improves the results of sequencing on the NovaSeq platform, with improved NextSeq concordance at the lower end of the assay. Experimental evidence showed that clean-up treatment using HPLC and FAB reagent treatment improved the results obtained in sequencing with NovaSeq, and improved NextSeq concordance at the lower end of the assay, compared to the use of FAB reagent alone or no treatment. In some embodiments, an Illumina library is fractionated by HPLC. Terminal Deoxy Transferase (TdT) and Dideoxynucleotide Triphosphates (ddNTPs)

In certain embodiments, the relative concentration of extendable free and/or buried primers is reduced using terminal deoxy transferase (TdT) to add dideoxynucleotide triphosphates (ddNTPs) to the 3′ end of the free and/or buried primers (and incidentally the amplification product itself), thereby preventing further elongation of the primers and preventing index hopping.

TdT adds ddNTPs to the ends of free primers, preventing their elongation. However, TdT works best at 37° C., which means it is ineffective under most denaturing conditions. Thus in certain embodiments, the TdT reaction is performed in the presence of a reagent that is capable of freeing buried primers (e.g., a protein that is capable of freeing buried primers). Examples of proteins that can be used to free buried primers include, but are not limited to, single-strand binding protein (SSB), recA, and UvrD. In certain embodiments, such reagents free buried primers, facilitating the addition of ddNTPs to their 3′ end by TdT.

Thus, in some embodiments, the relative concentration of extendable free and buried primers can be reduced by incubating the amplification product with TdTs, ddNTPs, and a reagent that can free buried primers under conditions amenable to TdT activity.

### Scavenger Nucleic Acid Molecules

In certain embodiments provided herein, index hopping is prevented by adding scavenger nucleic acid molecules to the sample comprising indexed sequencing template prior to sequencing. As used herein, a “scavenger nucleic acid molecule” or “scavenger nucleic acid” refers to a nucleic acid molecule that comprises: (A) a primer targeting region, which has a nucleic acid sequence complementary to a nucleic acid sequence at the 3′ region of a primer; and (B) a region having an irrelevant sequence that will not base-pair with the primer or an indexed sequencing template, wherein the region (B) is positioned 5′ to the region (A). In some embodiments, a scavenger nucleic acid molecule is single-stranded.

Thus, prior to or after loading the sample onto the sequencer, the scavenger nucleic acid molecules will hybridize to free primers and a DNA polymerase (e.g., Taq DNA polymerase) will extend the primer using a scavenger nucleic acid molecule template, resulting in extended primers that can no longer extend off normal templates due to the presence of the irrelevant sequence (FIG. 5).

The extension does not cause downstream data analysis problems as the irrelevant sequence signals can be later filtered out of the data set. Moreover, thermal cycling-based amplification can be used to release buried primers and extend them on the scavenger nucleic acid molecule template.

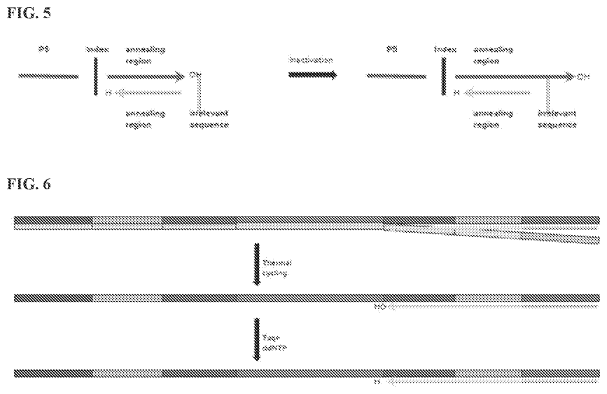

In some embodiments, a DNA polymerase can be used to extend the primer with ddNTPs, thereby further contributing to the neutralization of free primers. Because Taq DNA polymerase is compatible with thermocycling, this process can be used to inactivate buried primers (thermal cycling allows for buried primers to be re-annealed as primer:template complexes) (FIG. 6).

### Killer Oligonucleotides

In certain embodiments provided herein, index hopping is prevented by ligating free and/or buried primers to an oligonucleotide that prevents its further extension (a “killer oligonucleotide”).

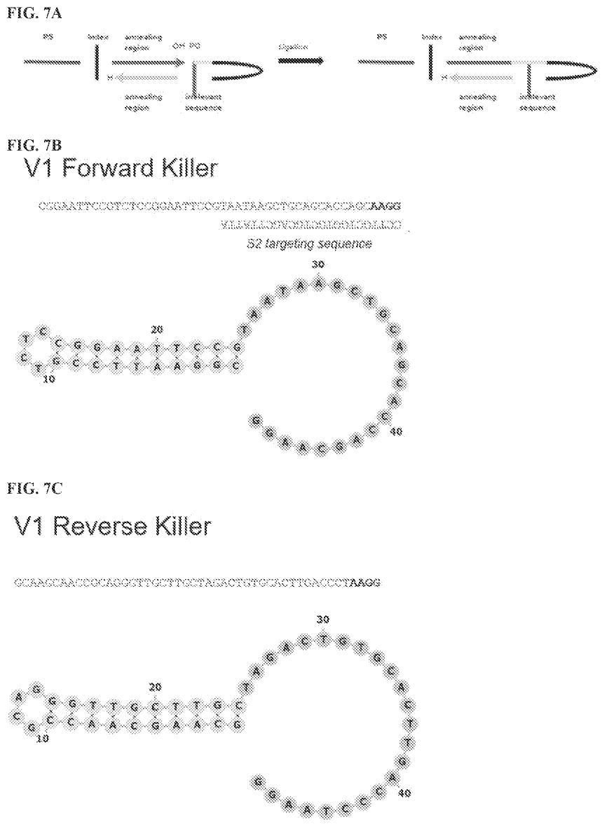

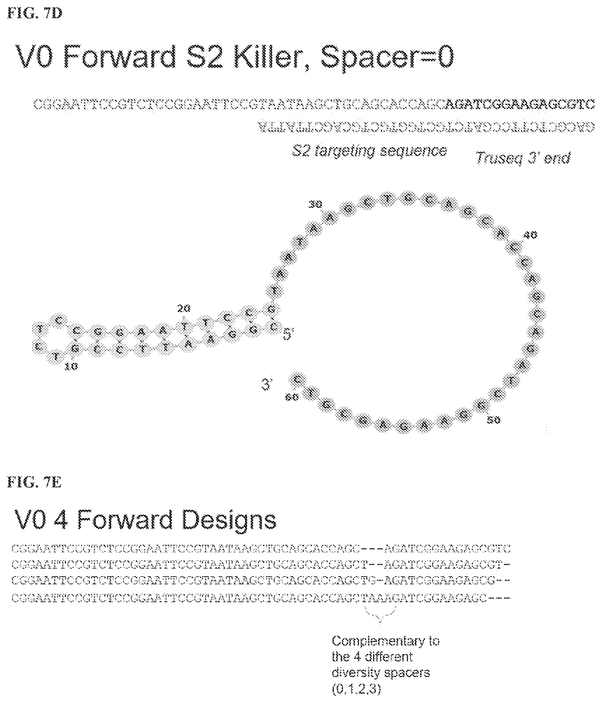

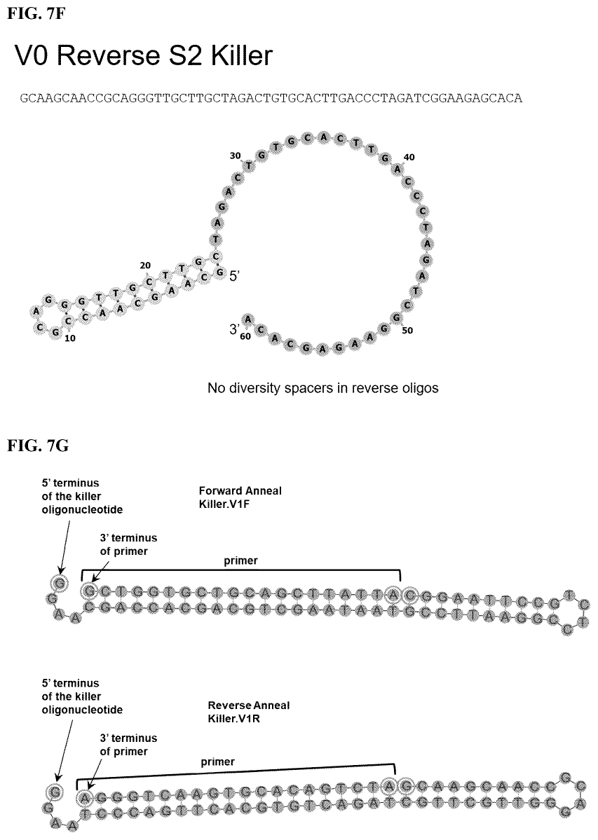

In certain embodiments, a killer oligonucleotide comprises a region having a sequence capable of hybridizing to the 3′ end of a primer, and when the primer is hybridized to the killer oligonucleotide, the primer can be ligated to the killer oligonucleotide. In certain embodiments, killer oligonucleotides are designed to have a structure that comprises a stem/loop region that has a sequence that forms a stem/loop structure positioned 5′ of a primer targeting region that has a sequence capable of hybridizing to the 3′ end of a primer (non-limiting examples are illustrated in FIGS. 7A-7F). In certain embodiments, a killer oligonucleotide comprises, in order from the 5′ end to the 3′ end: (A) a first region, (B) a second region, (C) a third region, and (D) a fourth region, wherein the first region (A) is capable of annealing (e.g., forming a duplex) with the third region (C), with the second region (B) forming a loop, such that the first region (A), the second region (B) and the third region (C) together form a stem/loop structure; and the fourth region (D) is a primer targeting region which is capable of hybridizing to (e.g., is complementary to) a region at the 3′ end of a primer. In some embodiments, when the primer is hybridized to the killer oligonucleotide, the primer and regions (A) to (D) are configured such that the base at the 3′ terminus of the primer and the base at the 5′ terminus of the first region (A) are adjacent to each other, such that the region at the 3′ terminus of the primer and the 5′ terminus of the first region (A) can be ligated to each other. In some embodiments, a killer oligonucleotide optionally comprises a fifth region (E) which is not capable of hybridizing with the primer or an amplification product (e.g., an irrelevant sequence). In certain embodiments, each of the regions (A), (B), (C), (D). and (E) are each independently about 5 to 40 bases long. In certain embodiments, each of the regions (A), (B), (C), (D), and (E) are each independently about 10 to 30 bases long. In certain embodiments, a killer oligonucleotide comprises a 5′ phosphate. In some embodiments, hybridization of a primer targeting region to a primer brings the 3′ terminus of the primer into proximity with the phosphorylated 5′ terminus of the killer oligonucleotide, thereby facilitating their ligation to each other by a ligase (FIG. 7G). In some embodiments, the killer oligonucleotide further comprises an irrelevant sequence (e.g., a sequence of oligonucleotides that will not base-pair with the primer or an indexed sequencing template) on its 3′ end to prevent it from being extended. In certain embodiments, the killer oligonucleotide comprises a ddNTP on its 3′ end to prevent it from being extended (e.g., as described above as region (E)). Non-limiting examples of a killer oligonucleotide comprising an irrelevant sequence on its 3′ end are illustrated in FIG. 7G, wherein the region at the 3′ terminus of the primer is shown in red, and the killer oligonucleotide is shown in green, a red circle indicates a ddNTP, and a blue circle represents a phosphorylated position.

In certain embodiments, a ligase is capable of ligating the 3′ terminus of the primer to the phosphorylated 5′ terminus of the killer oligonucleotide. In certain embodiments, the ligase is a thermostable ligase, such as Taq ligase. Thus, in certain embodiments, the sample comprising the amplification product is heated (e.g., to at least 85° C.) in the presence of killer oligonucleotides to release buried primers. The sample is then cooled to allow the free and previously buried primers to hybridize and ligate to the killer oligonucleotides, thereby neutralizing them. In some embodiments, the structure of the killer oligonucleotide can comprise the structure and/or sequence of any one of the killer oligonucleotides illustrated in FIGS. 7B-7F.

### Biotinylated Primers

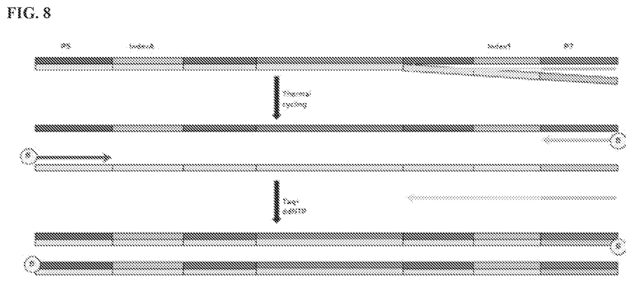

In certain embodiments, other techniques can be used to separate indexed sequencing template from free and buried primers. For example, in certain embodiments, biotinylated primers are used to perform an additional amplification reaction on the indexed sequencing template to generate a biotinylated amplification product. For instance, as illustrated in FIG. 8, single cycle of PCR with biotinylated primers (e.g., P5 and/or P7 primers), followed by binding the resulting biotinylated amplification products to streptavidin beads and denaturing can be used to purify the template strand from free and/or buried primers prior to sequencing.

### Sequencing, Determining Results, and Communicating Results

In certain embodiments, the methods and compositions disclosed are compatible with multiple sequencing platforms. One of ordinary skill in the art will know how to modify a primer, template, or reaction conditions to be compatible with other sequencing methodologies or those methodologies that come online in the future. For example, the sequencing component of the diagnostic assays disclosed can be performed using commercially available platforms such an Illumina or IonTorrent platform. Other platforms are contemplated as well.

In some embodiments, the NGS Modality is any of the following: SwabSeq, 1 Amplicon, 384 well plate, 96 Nextera barcode set, UDI's, NextSeq; SwabSeq—1 Amplicon, 384 well plate, 384 Truseq UDI barcode set, using NextSeq; or SwabSeq—1 Amplicon, 384 well plate, 4000 UDI Truseq barcode set, NovaSeq. SwabSeq—Multiplex, 384 well plate, CDI barcode set, NovaSeq.

For example, samples can be run on both NextSeq and NovaSeq. In certain embodiments, evaluation of the quality of the sequence data generated include analyzing the read counts and the fraction of reads that are used from the assay; the latter is a proxy for the load of non-productive artifactual products such as primer-dimers. In some instances, PCR artifacts can be index-specific, and the data analysis can identify those DNA barcodes that consistently perform badly, or at least worse than other DNA barcodes.

In some embodiments, the methods and compositions disclosed are designed to overcome other issues that can undermine a sequencing-based diagnostic assay. For example, Index hopping is another concern in NGS platforms (e.g., Illumina platforms such as NovaSeq), wherein reads are generated that have incorrect DNA barcodes relative to the true sample origin. The major cause of index hopping is believed to be free primers carried over from PCR during cluster generation. The “exclusion amplification” (ExAmp) technology used with patterned flowcells on the Illumina NovaSeq (as well as several other models) is particularly prone to index hopping events. ExAmp is a form of Recombinase Polymerase Amplification; instead of thermocycling, a combination of proteins enables primers to invade duplexes and be amplified by a strand-displacing DNA polymerase.

The ExAmp reagent is highly viscous. In some embodiments, the DNA library pool is denatured prior to mixing with ExAmp reagent, and is then added to the flowcell. The seeding of a nanowell on the patterned flowcell with a single library molecule will initiate an isothermal amplification process that rapidly consumes all of the surface-bound primers within that nanowell. Hence, if arrival of library molecules to the nanowells is an infrequent process, then each well will be “taken over” by the first library molecule to arrive before a second library molecule can enter.

If a stray primer binds to the library primer prior to entering a nanowell, that primer can be extended by the ExAmp reagents and generate a copy which replaces one original index sequence on the molecule with the stray primer's index sequence. This process can potentially be repeated due by the ExAmp reagents being capable of allowing primers to invade duplexes and be extended. If fragments from such grafting seed a well, then clusters (and hence reads) will result with index swaps.

Reductions in index hopping enhance limit-of-detection and robustness. In some embodiments, index hopping reduction can be accomplished by purifying the library mixture prior to loading on the sequencer. A proprietary Illumina enzymatic reagent, Free Adapter Blocking Reagent (FAB) and/or high-performance liquid chromatography (HPLC) can be used to purify samples or libraries of samples.

In some embodiments of the present disclosure, a unique dual indexing (UDI) strategy is employed, wherein the primers are used in pairs and the primers comprise unique, non-redundant indices (e.g., barcodes). This strategy reduces but does not eliminate the possibility of index hopping, a variety of index misassignment that results in incorrect assignment of libraries from the expected index to a different index in the pool. The mechanism of index hopping is believed to be largely driven by indexing primers or unified primers. This issue is a major cause of increases in index misassignment observed in sequencing using patterned flow cells. Index hopping at a minimum wastes data, as with a UDI scheme a single hopping event will create an “illegal” DNA barcode combination that does not correspond to any sample, wherein illegal barcode combinations are disregarded. Most dual hopping events should also create illegal combinations, though the possibility of a dual hop creating a legal code (both hops for the same sample in the UDI scheme) is not impossible. In some embodiments, a dual hop creating a legal code which corresponds to any particular patient's sample can produce a false positive, and this incorrect result can then be unfortunately communicated to the patient. In some embodiments, while using a unique dual indexing strategy (as opposed to, for example, using only a single indexing strategy) can reduce index, even a small amount of index hopping can result in false positives and/or false negatives. Various processes and compositions are described herein for further reduction of index hopping, even when unique dual indexing is used.

In some embodiments, the amplicons generated during a method provided are sufficiently long such that there is a substantial size difference between the true amplicons and the most likely types of PCR artifacts. In some embodiments, the size difference allows for better separation by both solid phase reversible immobilization (SPRI) and HPLC, enabling a higher fraction of assay reads by depleting PCR artifacts. In some embodiments, an aggressive SPRI purification is used, which reduces the load of free primers and hence index hopping.

In certain embodiments, processing of the sequencing data comprises demultiplexing the sequencing reads, processing the reads to remove systematic errors, low quality regions, and adapter sequences, and generating alignments and read counts. The NGS pipeline then runs to consolidate sample identifiers, properties, and analysis read counts into an output file (FIG. 9).

### Reagents and Equipment

As is understood by one of ordinary skill in the art, various different reagents and pieces of equipment can be readily obtained from various vendors and can be readily substituted in any method provided herein.

Non-limiting examples of various reagents and equipment suitable for use in a method of the present disclosure include but are not limited to:
Binding Solution (catalog number: A42359)
Wash Buffer (catalog number: A42360)
Elution Solution (catalog number: A42364)
Proteinase K Solution (catalog number: A42363)
Binding Beads (catalog number: A42362)
P20 LTS Tips, Rainin: part number: 17014399
P200 LTS Tips, Rainin, part number: 17014402
P1000 LTS Tips, Rainin, part number: 17007081
P1000 Pipettor
P10 multi-channel pipettor
250 mL Centrifuge Tube, Corning, part number 430776
10 mL Reservoir, Integra, part number
25 mL Reservoir, Integra, part number: 4352
100 mL Reagent Reservoir, VWR: part number 1346-1010
80% Ethanol, americanBio, part number: AB04091-01000
2 mL Deep Well 96-Well Microplate, Costar, part number:
Deep Well 96-Well Microplate, Eppenorf, part number: 951033006
Deep Well 24-Well Plate, Axygen, part number: 14222350
UltraPure DNase/RNase-Free Distilled Water: Invitrogen, part number: 10977015
Platemax Clear Plate Seals, Axygen: part number: PCR-TS-900
96 Deepwell HTS Reservoir, Thomas Scientific: 1171H96
j FP, Eppendorf: model 5385
Magnet Plate, Alpaqua: part number A001322
P-200 Liquidator, Rainin: part number LIQ-96-200
Proteinase K, 20 g/L stock solution (Thermo, PN: 25530049)
1,4-dithiothreitol (DTT) powder (Sigma Aldrich, PN: 43819)
Water for Proteinase K/DTT solution (Invitrogen 10977015)
Binding Solution (ThermoFisher Scientific A42359)
Binding Beads (ThermoFisher Scientific A42362)
Proteinase K Solution (ThermoFisher Scientific A42363)
Wash Buffer (ThermoFisher Scientific A42360)
80% ethanol (americanBio AB04091-01000)
Elution Solution (ThermoFisher Scientific A42364)
Nuclease-free water for RNA elution (Invitrogen 10977015)
Hamilton Starplus for Sample consolidation

Non-limiting examples of various instruments which can be or have been used in a method of the disclosure include:
The Concentric by Ginkgo SARS-CoV-2 NGS assay can be used with an RNA extraction procedure using the MagMAX™ Viral/Pathogen Nucleic Acid Isolation Kit (Applied Biosystems/ThermoFisher Scientific).
RT-PCR can be performed using a Labcyte Echo 525 liquid handler for reagent transfer and an Eppendorf Mastercycler x50t for thermocycling.
Library pools can be purified using AMPure XP reagent (Beckman Coulter), quantified by a KAPA Library Quantification Kit using a Roche LightCycler® 480 II and Quant-iT™ dsDNA Assay Kit, broad range (Invitrogen) using a BioTek Neo 2 Synergy Plate Reader, and visualized using an Agilent 2100 Bioanalyzer.
The purified library pools can be sequenced using an Illumina NextSeq 500 (software version 2.2.0).

Non-limiting examples of software which can be or have been used in a method of the disclosure include:
Base calls are converted to sequence reads and demultiplexed using bcl-convert (version v00.000.000.3.5.3-80-gdb27fdd9).

The sequence reads can be trimmed using Trimmomatic v0.36. Trimming is performed with the following settings and their impact described:
MINLEN: 16—any read less than 16 bases is discarded.
HEADCROP: 3—the first 3 bases of each read are removed. This removes any diversity spacers from the read.
ILLUMINACLIP: adapters.fa: 2:30:10—Illumina sequencing adapters to be removed. 2 indicates that a mismatch of 2 bases will be allowed between an adapter and a sequence for a match; 30 only applies to paired end reads and has no impact here; and 10 is the minimum threshold for alignment between the read and an adapter sequence.
LEADING: 30—any bases with a quality score below 30 at the beginning of the read will be trimmed.
TRAILING: 30—any bases with a quality score below 30 at the end of the read will be trimmed.
SLIDINGWINDOW: 4:20—from left to right, taking the average quality score of 4 bases, if that score drops below 20, the bases following that position are removed.

Following trimming, reads can be aligned to target reference sequences using Bowtie2 (version 2.4.1) with the parameters:
D 20—The number of attempts to perform an extension of a matching seed sequence before failing. This controls how thoroughly bowtie2 attempts to find an optimal alignment
L 7—the length of the seed for alignment to the reference sequences
i S,1,0.50—this guides how the seed sequences are generated from a read

Alignments can then be filtered using samtools (version 1.9) to remove any alignments with a mapping quality below 20, and alignments that do not fully span a required region (including 5 of the 6 bases of the spike in sequences and the previous 7 bases) are excluded using bedtools (v 0.25.0). Three reference sequences can be used for alignment: 1) SARS-CoV-2 S gene, 2) S spike-in internal control, and 3) human RPP30 gene sequences. The S gene reference sequence is derived from the NC_045512.2 genome. Alignment to the SARS-CoV-2 S reference sequence with these parameters ensures that the aligned sequence reads correspond specifically to the SARS-CoV-2 genome. Transcript counts can be generated by running the samtools (version 1.9) idxstats command to generate the read counts per transcript.

### EXAMPLES

### Example 1: Index Hopping Treatment Efficacy

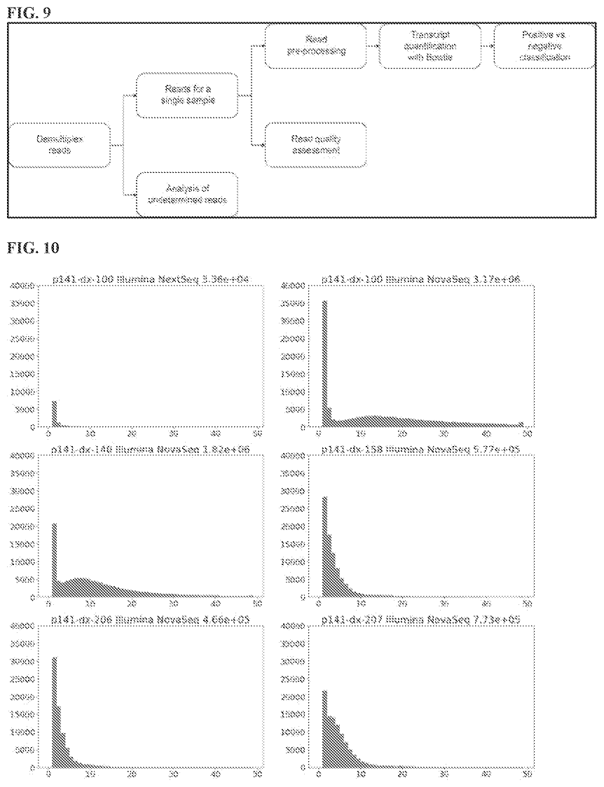

Multiple approaches are provided herein for reducing the prevalence of index hopping, which can result in improperly indexed sequencing reads and produce false results and therefore contaminate data and reduce the power of the assay. Five different approaches to reduce index hopping were evaluated. Referring to FIG. 10, the different methods were evaluated and compared to sequencing results obtained from untreated NextSeq reactions (top left panel) and untreated NovaSeq reactions (top right panel). As evident from the graph for the NextSeq reactions, which involves PCR amplification of templates, this sequencing protocol is less subject to index hopping. The middle left panel shows data from NovaSeq reactions that were generated after treatment with an example scavenger nucleic acid molecule provided herein. Scavenger nucleic acid molecule treatment causes a left-shift relative to untreated NovaSeq.

Such a scavenger nucleic acid molecule-based method is fast, easy, and compatible with other methods, allowing multiple treatment approaches to be combined with improved results. For example, NovaSeq reactions subjected to a HPLC purification process described herein in combination with FAB treatment and scavenger nucleic acid molecule treatment exhibited a more pronounced left shift and less area under the curve compared to scavenger nucleic acid molecule treatment alone (middle right panel) The bottom left panel illustrates the results of treatment with TdT and ddNTPs in the presence of SSB in combination with a scavenger nucleic acid molecule. Finally, the bottom right panel illustrates the results of treatment with a killer oligonucleotide and Taq ligase in combination with a scavenger nucleic acid molecule.

### Example 2: HPLC+FAB

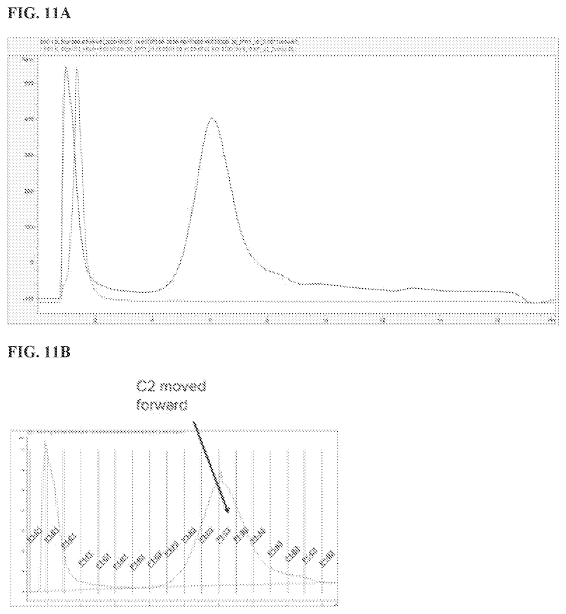

High Performance Liquid Chromatography (HPLC) purification of was performed using Ion-Pairing Reverse Phase (IP-RP) chromatography. This technique separates DNA oligonucleotides based on size, allowing isolation of longer PCR products from contaminating primers of shorter length. HPLC purification was followed with Illumina's FAB reagent treatment. Referring to FIG. 11A, the two peaks in the representative chromatogram of a library correspond to primers (left-most peak) and single-stranded amplicons (right-most peak). 1-2 fractions likely to contain mostly single stranded amplicon are typically used for sequencing (FIG. 11B).

### Example 3: Taq+ddNTP

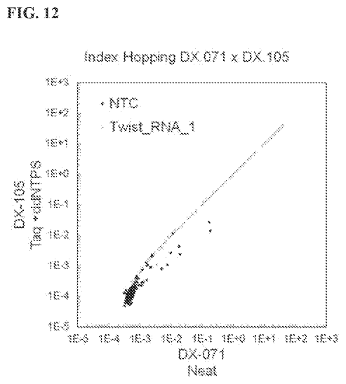

Index hopping was compared between untreated samples (DX-071) and samples treated with Taq DNA polymerase and ddNTPs (DX-105). Referring to FIG. 12, the treated samples resulted in fewer No Template Controls (NTCs) above threshold (S-ratio=1e-3), thereby demonstrating reduced index hopping in the treated samples relative to the untreated samples.

### Example 4: Reducing Primer Concentration

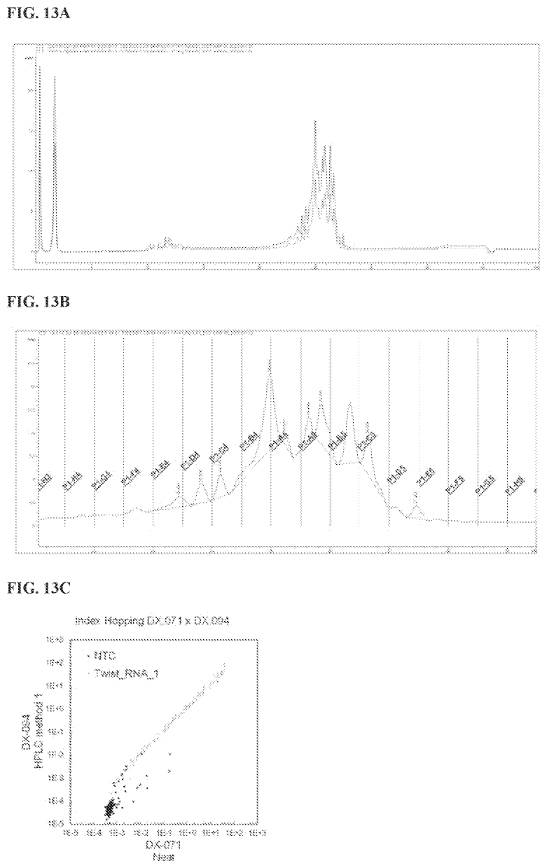

Reducing primer concentration was examined as a potential means of reducing free primers carried over from amplification that may result in index hopping. HPLC under denaturing conditions was used to separate amplicon single-strand DNA (ssDNA) from primer ssDNA. The denaturing conditions included pH=12 (long method) Ion-Exchange chromatography (FIGS. 13A, 13B). Ion exchange chromatography separates based on charge, so even though column retention should trend with increasing size, there are several more factors that dictate interaction with the column (i.e., GC content, secondary structure, etc.). There is a drastic decrease in separation efficiency above 80-90 nucleotide fragments. These columns are mainly used for n/n+1 separation of nucleic acids <80 nucleotides. There are fewer data points for NTC samples (8) above the 1e-3 S-ratio for DX-094 as compared to DX-071 (20), which did not employ HPLC (FIG. 13C).

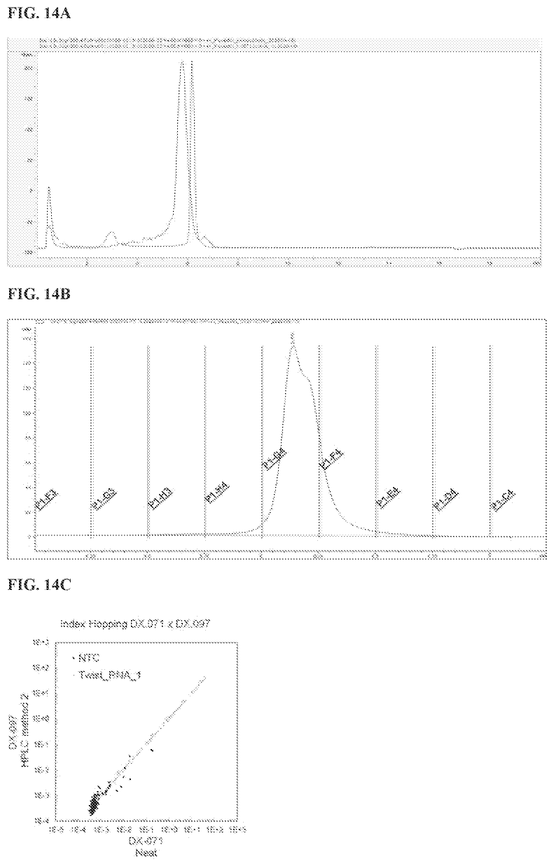

A method employing HPLC under denaturing conditions (pH=12) using ion exchange chromatography columns with a shortened run time. This method essentially condensed the numerous peaks observed in FIGS. 13A and 13B into a single peak (FIGS. 14A, 14B). There are more data points for NTC samples (29) above the 1e-3 S-ratio for DX-097 as compared to DX-071 (20), which did not employ HPLC (FIG. 14C).

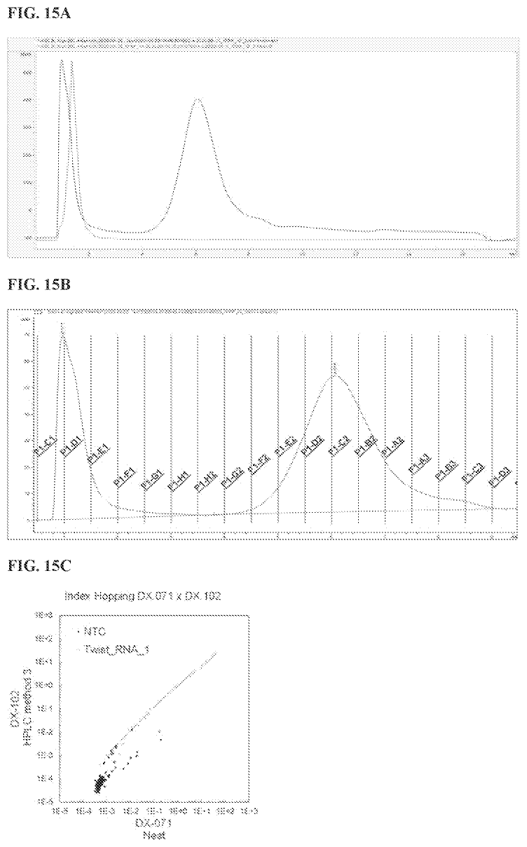

HPLC using denaturing conditions (85° C.) and ion-pairing reverse phase chromatography was also assessed for the ability to separate amplicon ssDNA from primer ssDNA. These HPLC conditions give more of a true size-based separation of nucleic acids. An ion-pairing reagent (triethylammonium acetate in this case) neutralizes the charge of the nucleic acid fragments, which allows the fragments to engage in hydrophobic interactions with the column for a more traditional reverse phase HPLC approach. As shown in FIGS. 15A and 15B, single peaks were obtained that allowed separation of primers from amplicons. Additionally, there were fewer data points for NTC samples (9) above the 1e-3 S-ratio for DX-094 as compared to DX-071 (20), which did not employ HPLC (FIG. 15C).

### Example 5: Primer Concentrations

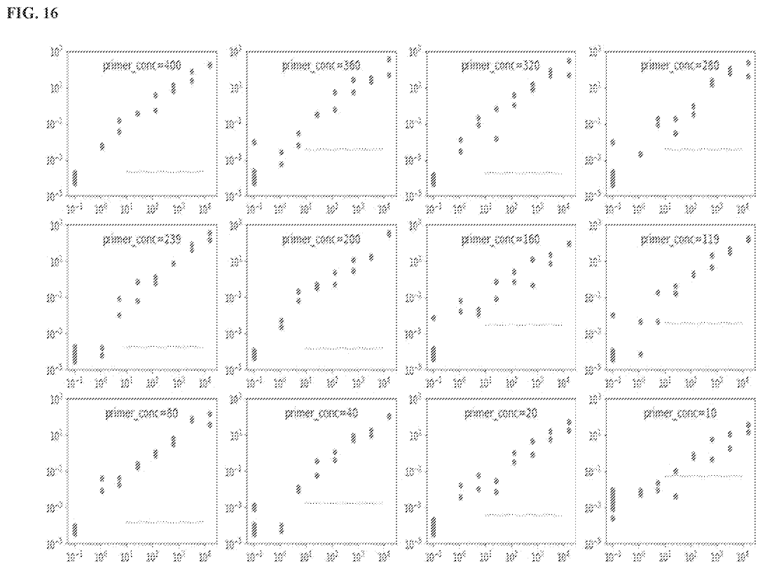

To determine if primer concentration affect index hopping, varying concentrations of primers were used in. FIG. 16 shows the prevalence of index hopping for different concentrations of the primer, suggesting that this is a variable to consider when designing sequencing experiments to minimize index hopping.

### Example 6: Various Protocols

One of ordinary skill will understand the method of the disclosure can be performed using various steps, protocols, reagents, equipment, etc., described and/or known in the art. This example describes various non-limiting examples of various protocols that can be used in a method of the disclosure.

### Example 6A. A Method for Detecting a SARS-CoV-2 Nucleic Acid Molecule in a Sample

The following non-limiting examples of protocols were and can be used in a method of detecting a SARS-CoV-2 nucleic acid molecule in a sample.

A method was performed with the following parameters, including controls: 90 μL of 1) pooled treated saliva, 2) pooled untreated saliva, or 3) water was mixed with 10 μL of diluted heat-inactivated SARS-CoV-2 (ATCC® VR-1986HK™). RNA extraction was conducted following 1) an automated procedure utilizing SPEEDBEADS™ magnetic carboxylate modified particles, sold by MilliporeSigma, St. Louis, MO; or 2) MagMax RNA extraction kit. N1 primer/probe mix and TaqPath™ 1-Step RT-qPCR Master Mix, CG (A15299) were used to set up reaction in 20 μL final volume. 5μL of RNA extracted samples were stamped to Roche 384-well white plate. Synthetic SARS-CoV-2 RNA Control 1 from Twist (LOCATION) and ATCC Heat-inactivated SARS-CoV-2 were used for calibration curve. RT-qPCR reaction was conducted using LightCycler480 (DEFINE, COMPANY, LOCATION) following protocol (RT—55° C./10 minutes; denature—95° C./1 minute; denature—95° C./10 seconds and extension—60° C. 30 seconds with plate read—40 cycles). Samples were then analyzed with NGS (next generation sequencing), using either NextSeq or NovaSeq.

Untreated saliva samples extracted through automation led to very low sensitivity (dropouts could suggest pipettability issues). Manual RNA extraction using MagMax kit showed consistent results without dropouts across RNA matrices conditions up to 800 copies/mL. All controls are valid. Extractions using MagMax kit resulted in a greater number of positive samples at lower viral-RNA concentrations compared to Ginkgo automated extraction method.

The following results were obtained from extraction method tests:
General LoD (>95% of samples classified as positive)
MagMax manual extraction—>1,600 copies/mL
Automated extraction—>>25,600 copies/mL
ATCC virus outperforms spike-in calibration line (as expected from non-synthetic RNA)
Experiment specific observations for one experiment (Automated extraction method):
Pooled saliva samples (untreated) had lower log-s-ratio especially in higher end of viral RNA concentrations.
A given sample performed comparably in both NextSeq and NovaSeq platforms when the samples were close to or over the positive classification threshold.
Experiment specific observations for one experiment (MagMax extraction method): A given sample performed comparably in both NextSeq and NovaSeq platforms when the samples were close to or over the positive classification threshold.

### Example 6B. Preparation of Sample, Including RNA Extraction

Provided below is a non-limiting example of a protocol for preparation of a sample, including RNA extraction.
1. Prepare sample—Aliquot 100 μL of Saliva in MTM treated with DTT and proteinase K into each well of a Costar 3798 round bottom plate (e.g., done by hand prior to loading onto Hamilton).
2. Mix the bottle of 1×GE beads to fully resuspend the beads. To prepare 1×GE beads mix 12.5 mL beads (Sera-Mag Speedbeads by GE—65152105050350) in 500 mL of buffer (Pura Buffer by americanBio—CU06300-00500).

Start of automated protocol on Hamilton:
3. Add 100 μL of beads to the samples in a Costar 3798 round bottom plate and pipette mix 15 times with a mix volume of 150 μL. Incubate samples at room temperature (15-30° C.) for 10 minutes.
4. Place the processing plate on the Magnet Plate (Alpaqua A001322) and incubate at room temperature for 5 minutes to allow beads to separate.
5. Fully remove supernatant from the processing plate and discard. This step must be performed while the processing plate is situated on the magnet.
6. Leave the processing plate on the magnet and wash by adding 150 μL of 70% ethanol to the sample. Allow samples to sit with ethanol for 1 minute.
7. Completely remove supernatant from the processing plate and discard. This step must be performed while the plate is situated on the magnet. Do not disturb the ring of separated magnetic beads.
8. Repeat steps 6-7 for a total of 2 washes.
9. After final wash, replace any remaining 300 uL tips with 50 uL tips and remove any remaining liquid using 50 μL FTR tips.
a. A dialog box will appear before the first 50 uL transfer reminding the user to place the correct tips in the FTR carrier.
10. Allow magnetic beads to dry for 5 minutes at room temperature (15-30° C.). Beads do not need to be completely dry, but the traces of liquid should be gone (i.e., droplets or puddles).
11. Remove the processing plate from the magnet. Elute nucleic acid by adding 50 μL of nuclease-free water and pipette mixing 10 times.
12. Return the plate to the magnet for 2 minutes and carefully transfer 45 μL of eluted nucleic acid away from the beads and into a fresh 96-well PCR plate for storage.

In the protocol of Example 6A, or in another method for preparation of the sample, including RNA extraction, the following parameters are used:
Equipment: Hamilton STAR Plus
Capacity: 4 96-well plates per run
Time to completion: ˜1 hr for 4 plates
Reagents: Sera-Mag Speedbeads (GE, 65152105050350), Pura Buffer (AmericanBio, CU06300-00500), 80% ethanol (AmericanBio), Nuclease free water
Consumables:
   Plates:
      Costar 3798 round bottom plates, Eppendorf 96-well PCR plates, Alpaqua ring magnet plates (A001322)
   Tips:
      300 uL FTRs with filter, 50 uL FTRs with filter
   Troughs:
      Automation Reservoirs (Thermo, 1064 May 6)

### Example 6C. An Alternative Method for Preparation of the Sample, Including RNA Extraction

Provided below is a non-limiting example of a protocol for preparation of a sample, including RNA extraction, wherein the protocol involves the use of the MagMAX™ Viral/Pathogen Nucleic Acid Isolation Kit.

This protocol is derived from MagMAX extraction protocol (Pub. No. MAN0018072 Rev. B.0), as sets.thermofisher.com/TFS-Assets/LS G/manuals/MAN0018072_MagMAXViralPathoNuclAcidIsolatKit_Manually_UG. pdf

In some embodiments of this protocol, steps 1.a and 1.b are omitted or replaced by different steps.

Perform Total Nucleic Acid Purification Using 200-400 μL:
1. Digest with Proteinase K:
a. Add 10 μL of Proteinase K to each well of a Deep-well 96-well plate. This plate is the Sample Plate.
b. Add 200-400 μL of each sample to wells with Proteinase K in the Sample Plate. Use of up to 200 μL input for whole blood is recommended.
c. Invert Binding Bead Mix gently to mix, then add 550 μL to each sample in the Sample Plate. Remix the Binding Bead Mix by inversion frequently during pipetting to insure even distribution of beads to all samples or wells. The mixture containing the Binding Beads is viscous. Therefore, pipet slowly to ensure that the correct amount is added. Use of a repeat pipet to add to the samples as the high viscosity will cause variations in volume added and is not recommended.
d. Seal the plate with MicroAmp™ Clear Adhesive Film, then shake the sealed plate at 1,050 rpm for 2 minutes.
e. Incubate the sealed plate at 65° C. for 5 minutes (ensure the bottom of the plate is uncovered), then shake the plate at 1,050 rpm for 5 minutes.
f. Place the sealed plate on the magnetic stand for 10 minutes, or until all of the beads have collected.
2. Wash the beads:
a. Keeping the plate on the magnet, carefully remove the cover, then discard the supernatant from each well. Avoid disturbing the beads.
b. Remove the plate from the magnetic stand, then add 1 mL of Wash buffer to each sample.
c. Reseal the plate, then shake at 1,050 rpm for 1 minute.
d. Place the plate back on the magnetic stand for 2 minutes, or until all the beads have collected.
e. Keeping the plate on the magnet, carefully remove the cover, then discard the supernatant from each well. Avoid disturbing the beads.
f. Repeat step 2b to step 2e using 1 mL of 80% Ethanol.
g. Repeat step 2b to step 2e using 500 μL of 80% Ethanol.
h. Dry the beads by shaking the plate (uncovered) at 1,050 rpm for 2 minutes.
3. Elute the nucleic acids:
a. Add 50-100 μL of Elution Solution to each sample, then seal the plate with MicroAmp™ Clear Adhesive Film.
b. Shake the sealed plate at 1,050 rpm for 5 minutes.
c. Place the plate in an incubator at 65° C. for 10 minutes.
d. Remove the plate from the incubator, then shake the plate at 1,050 rpm for 5 minutes.
e. Place the sealed plate on the magnetic stand for 3 minutes or until clear to collect the beads against the magnets.

f. Keeping the plate on the magnet, carefully remove the seal, then transfer the eluates to a fresh standard (not deep-well) plate. To prevent evaporation, seal the plate containing the eluate immediately after the transfers are complete. The purified nucleic acid is ready for immediate use. Alternatively, store the plate at −20° C. for long-term storage.

### Example 6D. Sequencing

While different sequencing platforms exist, this example provides a general protocol that one skilled in the art can use to obtain quality results. First, the samples are subjected to a PCR amplification reaction. Subject samples and control samples are plated on a 384-well plate. The plate is spun at 4000 RPM for 3 min to collect each sample at the bottom of their respective wells. To these wells the following are added: 75 nL 80 μM S2 i7 primer (for a final concentration of 400 nM in the PCR reaction); 75 nL 80 μM S2 i5 primer (for a final concentration of 400 nM in the PCR reaction); 25 nL 30 μM RPP30 i5 primer (for a final concentration of 50 nM in the PCR reaction); and 25 nL 30 μM RPP30 i7 primer (for a final concentration of 50 nM in the PCR reaction). The PCR plate is centrifuged at 4,680 RPM for 1 minute.

In a 5 mL tube, a master mix is prepared by adding S2 spike-in RNA at a concentration of 1×104 copies/μL, and TaqPath 1-Step RT-qPCR Master Mix, CG. Using the plate map as a guide, add Master Mix solution to each well containing a patient sample or control. The total reaction volume per well should be 15 μL. Note: Wells G23, 123, K23, M23, and O23 are not used and do not require Master Mix or sample. The plate is centrifuged at 4680 RPM for 1 min before removing the plate seal. 10 μL of master mix is added to each well containing primer. Using the plate map as a guide, the following templates are added: 4.8 μL of the patient samples (wells A1-H11) from the sample plate to their corresponding RT-PCR wells, which should already contain Master Mix and Primer; 4.8 μL of Positive control: high concentration (2000 copies Twist Control 1 /μL) to well A23; 4.8 μL of Positive control: low concentration (20 copies Twist Control 1/μL) to well C23; 4.8 μL of nuclease-free water to well E23. The plate is then sealed and centrifuged.

To create pooled samples, 5 μL of each sample is transferred from a 384-well plate (post RT-PCR) to a reservoir for pooling. The pooled libraries are well mixed before removing an aliquot for purification. 200 μL of the library pool is transferred to a 1.5 mL tube and label the tube. The remaining unused library pool is transferred to a 2 mL tube, label, and store at 4° C. The AMpure XP vial is vortexed thoroughly, then 160 μL of well-resuspended room temperature AMpure XP beads is added to the tube, incubated, and mixed at room temperature using the Hula for 10 minutes.

After mixing, the tube is briefly centrifuged for 3 seconds to collect the liquid to the bottom of the tube. The tube is placed on a magnetic stand for 5 mins to separate the beads from the solution. DNA larger than the desired size will bind to the beads. 340 μL of the supernatant is carefully transferred into a new 1.5 mL tube. The supernatant contains DNA that will be further processed for sequencing. 40 μL of AmpureXP beads is added to the new tube. DNA smaller than the desired size will remain in solution. This is incubated and mixed at room temperature using the Hula for 10 minutes.

After mixing, the tube is briefly centrifuged for 3 seconds to collect the liquid to the bottom of the tube. The tube is placed on the magnetic stand for 5 mins. DNA with the desired size is bound to the beads. The supernatant is removed and discarded. 200 μL of 80% EtOH is added and incubated at room temperature for 30 seconds (1st wash). The supernatant is removed and discarded. 200 μL of 80% EtOH is added and incubated at room temperature for 30 seconds (2nd wash). The supernatant is removed and discarded.

Any residual EtOH is carefully removed with a p20 pipette. Residual EtOH can inhibit downstream application. The beads are air-dried for 30 sec. Over-drying the beads will reduce DNA recovery. The tube is removed from the magnetic stand and 42 μL of Nuclease-free water is added and pipetted to resuspend. The beads are incubated at room temperature for 3 minutes (off the magnetic stand) and then placed on the magnetic stand for 3 minutes to separate the beads from the solution. 40 μL of the purified library is carefully transfer into a new 1.5 mL tube. The pooled library may be kept for up to 3 months at 20° C.

After pooling is completed, samples are amplified by RT-PCR using an Eppendorf Mastercycler x50t using the following steps: UDG decontamination: 25° C. for 2 minutes; Reverse transcription: 53° C. for 15 minutes; PCR enzyme activation: 95° C. for 2 minutes; 40 cycles of PCR: 95° C. for 15 seconds; 64° C. for 60 seconds; Hold at 10° C. indefinitely.

The RT-PCR plate may be kept in the thermocycler for up to 24 hours at 10° C.

After RT-PCR is completed, PicoGreen quantification is performed. A “BR working stock” is prepared by making a 1:200 dilution of Quant-iT dsDNA BR reagent in Quant-iT dsDNA BR buffer. 15 μL QuantiT dsDNA BR reagent+2985 μL Quant-iT dsDNA BR buffer i is prepared. A 1:10 dilution of the library pool in DNase/RNAse-Free Distilled Water for quantification is prepared. Both the undiluted and the 1:10 dilution will be used for quantification.

For each standard and to both the diluted and undiluted library, 98 μL of BR working stock is added per library and standards (24 wells for 8 standards each run in triplicate). Both the neat pool and the 1:10 dilution of the pool are quantified, and the value that falls within the range of standards (0-100 ng/μL) is used for downstream molarity calculation.

To each well containing BR working stock, 2 μL library or standard is added and the plate is sealed, shaken, and spun briefly. The seal is removed and read on plate reader using PicoGreen assay protocol. The raw fluorescence data is used to convert into dsDNA concentration (ng/μL).

For each standard, the 3 replicates included on the plate are averaged, and the slope and y-intercept calculated using the raw fluorescence data and known concentration value for the standards, and use this linear equation to calculate the concentration of the pool. The concentration of the library pool is recorded in ng/μL. The R\hat{ }2 value is recorded, and must be greater than 0.98 to pass. If the R\hat{ }2 value does not pass, the procedure is repeated.

### Example 6E. Bioanalyzer Visualization Instructions

Obtain a Bioanalyzer 7500 kit and incubate at room temperature for 30 minutes at RT. If the Bioanalyzer is unavailable, contact the TS for instructions on using the TapeStation D1000 as an alternative.

Place the 7500 DNA chip onto the chip holding/pressurizing platform.

Reagents must equilibrate to room temperature for 30 minutes before preparation. To prepare the gel dye mix, vortex the DNA dye concentrate for 10 seconds and spin down. Pipette 25 μL of the DNA dye concentrate into a tube of DNA gel matrix. Cap the gel matrix tube, vortex for 10 seconds, and transfer the full volume into the top compartment of a spin filter. Centrifuge for 10 minutes at 4,000 RPM. Discard the filter.

Add 9 μL of Gel-Dye mix to the priming well. Use a back filling pipetting method to ensure there are no bubbles. Remove any bubbles that are present.

Lock the syringe into place and ensure the silver mechanism at the top is in the top slot.

Press down until the clip holds the syringe in place.

Allow the chip to prime for 30 seconds and then release the syringe.

Wait 5 seconds for the syringe to depressurize.

Gently pull back until 1 mL.

Add 9 μL of gel-dye mix to the other gel wells (marked with G) using the back filling pipetting method.

Add 5 μL of marker to the ladder well and all sample wells.

Add 1 μL of Ladder to the ladder well.

Add 1 μL of the diluted library to sample well 1.

Add 1 μL of the neat library to sample well 2.

Vortex the samples using the chip vortexer to shake for 1 minute at 2400 rpm.

Make sure there are no bubbles in the wells. If bubbles are present, shake again or remove with a pipette tip.

While the vortexer is running, add water to the wash chip and place in the Bioanalyzer for ˜30 seconds.

Remove the wash chip and allow probes to dry for ˜5-10 seconds.

Add the DNA chip with samples to the Bioanalyzer.

Select the DNA 7500 assay.

After program finishes running, perform a smear analysis on samples:

On the right hand side, select Global and advanced from the drop down.

Scroll to Smear Analysis and double-click.

Click Add region.

Define a region with a start at “100 bp” and an end at “1100 bp.”

Click OK.

Record the average insert size for this region.

### Example 6F. qPCR Quantification

Use the worksheet to perform calculations, record completed steps, record reagent lot numbers, and instrument identifiers, as above.

If opening a new kit, make sure to add the full volume from the primer tube into the larger master mix vial before use. Aliquot standards into a set of strip tubes or a 96-well plate in numerical order with standard 1 in row A and standard 6 in row F.

Make qPCR master mix [960 μL of KAPA Sybr Fast qPCR Master Mix (2×) and 320 μL of H2O] and then transfer 16 μL of qPCR master mix into following wells:

A1-A6 b. C1-C6 c. E1-E6 d. G1-G3 e. I1-I3 f. K1-K3

In a 96-well plate or a 0.2 mL tube strip, add 98 μL of H₂O to well A1 (for each batch, you will want to set up a row with 98 μL in the first position (b1, c1, etc.) and then the remaining wells as described below).

Add 90 μL of H₂O to wells A2 through A7.

In a 96-well plate or a 0.2 mL tube strip, add 2 μL of library pool to well A1. Pipet to mix with 50% of the volume. This makes a 1:50 dilution of the sample.

Transfer 10 μL from well A1 to well A2. Pipet to mix 10 times with 50% of the volume. This makes a 1:500 dilution of the sample.

Transfer 10 μL from well A2 to well A3. Pipet to mix 10 times with 50% of the volume. This makes a 1:5,000 dilution.

Transfer 10 μL from well A3 to well A4. Pipet to mix 10 times with 50% of the volume. This makes a 1:50,000 dilution.

Transfer 10 μL from well A4 to well A5. Pipet to mix 10 times with 50% of the volume. This makes a 1:500,000 dilution.

Transfer 10 μL from well A5 to well A6. Pipet to mix 10 times with 50% of the volume. This makes a 1:5,000,000 dilution.

Transfer 10 μL from well A6 to well A7. Pipet to mix 10 times with 50% of the volume. This makes a 1:50,000,000 dilution.

Pipet 4 μL of Standard in triplicate into wells of qPCR plate. Pipet to mix

Standard 1: A1-A3 b. Standard 2: C1-C3 c. Standard 3: E1-E3 d. Standard 4: G1-G3 e. Standard 5: I1-I3 f. Standard 6: K1-K3

Pipet 4 μL of diluted sample in triplicate into wells of qPCR plate. Pipet to mix

1:500,000 dilution into wells A4-A6 b. 1:5,000,000 dilution into wells C4-C6 c. 1:50,000,000 dilution into wells E4-E6.

Seal qPCR plate with permanent optical seal. Make sure there are no bubbles in the wells.

Spin down at 4,680 RPM for 1 minute to remove bubbles.

Load qPCR plate into Roche Lightcycler 480.

In the Lightcycler software, click “Create New Experiment from Template”

Select the template called “NGS_workflow_qPCR” and name the qPCR run with the appropriate workflow ID from organick.

The LightCycler will run for about 35 minutes.

After the program completes, select “Analysis,” “Abs quant/fit points,” then highlight the table produced, click “Calculate.” This will calculate the Cp values for all of the wells. Right click on the table, export the data and save as “w #.txt”.

Paste the values from the qPCR data into the R1 cell the upper right hand corner of a PandA batching template Excel file configured to parse the data.

Correct the standards, intercept, slope and check efficiency coefficient based on the standard curve generated.

Record the R\hat{ }2 value.

The R\hat{ }2 must be greater than 0.98 to pass. If the R\hat{ }2 value does not pass, repeat the procedure.

Safe Stop. The quantified library may be kept for up to three months at 20° C. Libraries that have been stored for more than one (1) week, quantification should be repeated prior to sequencing; use the new values for loading the sequencer.

After quantification is completed, begin the sequencing portion of the workflow by obtaining the reagents defined in the “4. Sequencing” Sheet of WKS-GBCL-0001.

### Example 6G. Sequencing Procedure

Obtain a NextSeq High Output Reagent Kit from −20° C. and thaw in the prepared water bath.

Obtain a NextSeq High Output Flow Cell from 4° C. and incubate at room temperature for 30 minutes.

Thaw the HT1 buffer from the kit in the prepared water bath.

Obtain a NextSeq Buffer Cartridge.

Obtain a 2 N NaOH solution.

Prepare a 0.2 N NaOH solution by adding 90 μL of H₂O and 10 μL of 2 N NaOH to a 1.5 mL Microcentrifuge tube.

Normalize the library to 4 nM with water according to the calculations on the “Loading Calculations” tab.

In a 1.5 mL tube, add 5 μL of 4 nM library and 5 μL of 0.2 N NaOH. Vortex, spin down and allow to incubate at room temperature for 5 minutes.

Add 990 μL of HT1 buffer. Vortex and spin down. This makes a 20 pM library.

Transfer 117 μL of the 20 pM library to a new 1.5 mL tube.

Add 1183 μL of HT1 to the new tube with the 117 μL of 20 pM library. Vortex and spin down. This will make a 1.8 pM library.

Prepare 1.8 pM PhiX. In a 1.5 mL tube, add 2 μL 10 nM PhiX+3 μL 10 mM Tris-HCl+0.1% Tween 20.

Add 5 μL 0.2N NaOH. Vortex, spin down, and allow to incubate at room temperature for 5 minutes.

Add 990 μL of HT1 buffer. Vortex and spin down. This makes 20 a pM PhiX.

Transfer 117 μL of the 20 pM PhiX to a new 1.5 mL tube.

Add 1183 μL of HT1 to the new tube with the 117 μL of 20 pM PhiX. Vortex and spin down. This will make it a 1.8 pM PhiX.

Prepare a 1.8 pM library with 10% 1.8 pM PhiX: 130 μL 1.8 pM PhiX+1170 μL 1.8 pM library.

Load the entire 1.3 mL of 1.8 pM library+10% PhiX into the NextSeq cartridge.

At the NextSeq, select “Sequence,” and load the flow cell once the stage opens.

Select next then load the buffer cartridge into the Nextseq. Empty the waste bin.

Select next then load the reagent cartridge into the NextSeq.

Select load.

Fill out the Workflow information.

Skip Library ID

The Flow Cell should say “NextSeq High Output”

Select “Single End”

Input the following read cycle lengths: ‘Read1: 36’ ‘Read2: 0’

Input the following index cycle lengths: ‘Index1: 8’, ‘Index2: 8’

Select “Next” to begin the pre-run system checks.

“Select “Start” once the pre-run system checks have completed. If the sequencer does not pass the pre-sequencing checks, contact the TS.”

### Example 6H. Loading the NovaSeq

Set a sous vide to warm a water bath to 70° F.

Obtain a NovaSeq 100 Cycle Reagent Kit from −20° C. and leave it to thaw in the prepared water bath.

Obtain a NovaSeq Cluster Kit from −20° C. and leave it to thaw in the prepared water bath. By default, the sous vide will start a 4 hour times, but the kit will usually thaw completely in about 2 hours.

Obtain a NovaSeq SP Flow Cell from 4° C. and incubate for 30 minutes at room temperature.

Obtain a NovaSeq SP/S1/S2 Buffer Cartridge.

Obtain 1 M Tris-HCl, pH 8.5.

Obtain a stock of DNase/RNase-Free Distilled Water.

Dilute 1 M Tris-HCl, pH 8.5 to 400 mM: 2 mL Tris-HCl+3 mL DNase/RNase-Free Distilled Water

Normalize the library to 2.5 nM with water according to the calculations on “Loading Calculations” tab.

Obtain 10 nM PhiX.

Obtain a stock of 2 N NaOH.

Prepare a 0.2 N NaOH solution by adding 90 μL of water to 10 μL of 2 N NaOH.

In a new 1.5 mL microcentrifuge tube, dilute the 10 nM PhiX to 2.5 nM PhiX: 2.5 μL 10 nM PhiX+7.5 μL water.

Add 90 μL 2.5 nM normalized library to the 1.5 mL microcentrifuge tube containing 10 μL of 2.5 nM PhiX. This results in a 2.5 nM library with 10% PhiX.

Add 25 μL of freshly prepared 0.2 N NaOH.

Vortex, spin down, and incubate for 8 minutes at room temperature.

Add 25 μL 400 mM Tris-HCl. Vortex and spin down briefly. Add full 150 μL volume into a NovaSeq library tube.

On Instrument pick either side of Sequencer to run (A or B) and click sequence

Log into BaseSpace to set up the run.

Enter WF and information regarding Read length, Barcode length, etc.

Make sure to empty out old reagents from the NovaSeq and load new buffer, sbs cartridge, and cluster kit to commence.

Empty out filled waste containers and check the button on NovaSeq to confirm.

Start run and make sure all pre-run checks pass and the run starts on the instrument before you can leave the area.

### Example 61. Index Hopping Treatments

### High Performance Liquid Chromatography

Ion exchange HPLC uses solvents composed of 25 mM sodium hydroxide in 20 mM Tris·HCl buffer (roughly pH 12.0) with and without 2 M sodium chloride. Samples are run on an Agilent 1260 Infinity Series HPLC equipped with a Thermo Fisher DNAPac PA200 4×50 mm column kept at 30° C. After samples were injected onto the column, the target oligonucleotides are eluted using a gradient from 0.5 M to 1.1 M sodium chloride over 35 minutes (Long run method) or from 0.8 M to 1 M sodium chloride over 15 minutes (short run method). The eluted material was collected throughout. Detection of eluted material was accomplished using a multiple wavelength detector set to 260 nm and 280 nm.

Ion-Pairing Reverse Phase HPLC uses solvents composed of 100 mM triethylammonium acetate pH 7.0 buffer with and without 25% acetonitrile and is run utilizing a Thermo Fisher Vanquish Flex UHPLC equipped with a Thermo Fisher DNAPac-RP column kept at 100° C. After samples are injected onto the column, the target oligonucleotides are eluted using a gradient from 0% acetonitrile to 25% acetonitrile over a period of 10-15 minutes, and the eluting material is collected throughout. Detection of material is accomplished using a multiple wavelength detector set to 260 nm and 280 nm.

### SPRI

For the library purification, a Double SPRI AMPure magnetic bead clean-up is performed on the pooled libraries. This is a 0.6× clean-up followed by a 0.2× clean-up that allows for a size selection of a library with an average insert size close to 452 bp. 500 μl of each pooled library is transferred, by batch, to an Eppendorf DNA Lo-Bind 2 mL Microcentrifuge tube. The beads are brought to room temperature (30 min to equilibrate) and vortexed thoroughly. 300 μl of room temperature AMPure XP beads are added to the 500 μl aliquots of pooled libraries. And mixed by pipetting 10 times.

DNA and beads are incubated for 10 minutes on the Hula Mixer, during which time DNA will bind to the beads. Tubes are placed on a magnet for 5 minutes to allow for the DNA bound to beads to separate from the supernatant. 720 μl of supernatant is transferred to a new 2 ml Eppendorf DNA Lo-Bind Microcentrifuge tube, and 144 μl of AMPure beads are added to these new tubes. Pipette up and down 10× to mix (this is the 0.2× bead clean-up), incubated on hula mixer for 10 minutes, and placed on a magnet for 5 minutes. The supernatant is removed with a P1000 pipette and discarded. 1 ml of 80% ethanol is added to wash the beads and incubated for 60 seconds on the magnet. The ethanol is then removed and discarded. An additional 1 ml of 80% ethanol is added to wash the beads. The beads are incubated again for 60 seconds on the magnet, and the ethanol is removed and discarded. The beads are dried for about 2-3 minutes. Beads that are over-dried will exhibit excessive cracking. A P20 pipette is used to remove any excess ethanol that remains while the sample dries.

The sample is eluted in 105 μl of H₂O by pipetting up and down 15 times while the tube is off the magnet. The sample is eluted for about 5 minutes off the magnet, and then moved to the magnet. The beads are then separated for 2 minutes. 100 μl of eluted sample is transferred to a new microcentrifuge tube.

### FAB Treatment

Fab reagent is thawed at RT and then put on ice. When ready to use, the FAB reagent is mixed by inversion and centrifuged at 600×g for 5 seconds. 200 μl of library sample pool is added to a PCR tube, and 200 μl of FAB reagent is added to each PCR tube and mixed thoroughly by pipetting up and down. The tubes are centrifuged briefly to make sure all contents are on the bottom of the tube and then incubated on a thermal cycler running the FAB program: 38° C. for 20 m; 60° C. for 20 m; and hold at 4° C.

### 2.5×SPRI

100 μl of each pooled library is transferred, by batch, to an Eppendorf DNA Lo-Bind 2 mL Microcentrifuge tube. The beads are brought to room temperature (30 min to equilibrate) and vortexed thoroughly. 250 μl of room temperature AMPure XP beads is added to the 100 μl aliquots of pooled libraries and mixed 10 times by pipetting. DNA and beads are incubated for 10 minutes on the Hula Mixer to allow binding. The tube is placed on a magnet for 5 minutes, which allows the DNA bound to beads to separate from the supernatant. The supernatant is removed with P1000 pipette and discarded. 500 μl of 80% ethanol is added to wash the beads and incubated for 60 seconds on the magnet. The ethanol is removed and discarded. An additional 500 μl of 80% ethanol is added to further wash the beads and incubated for 60 seconds on the magnet. The ethanol is removed and discarded. The beads are dried for about 2-3 minutes. The beads are closely watched to ensure they are not excessively cracking, which is an indication of over-drying. A P20 pipette is used to remove any excess ethanol that remains while the sample dries. The sample is eluted in 50 μl of H₂O by pipetting up and down 15 times while the tube is off the magnet. The sample is eluted for about 5 minutes off the magnet and then moved back to the magnet to allow the beads to separate for 2 minutes. 50 μl of eluted sample is then transferred to a new microcentrifuge tube.

QC and Loading with Scavenger

Scavenger nucleic acid molecules are stored as 100 μM stocks and mixed in equal parts. 4 μl mixed scavenger nucleic acid molecules are mixed with the purified library and loaded as normal.

### EQUIVALENTS

Those skilled in the art will recognize, or be able to ascertain using no more than routine experimentation, many equivalents to the specific embodiments of the disclosure described. Such equivalents are intended to be encompassed by the following claims.

### INCORPORATION BY REFERENCE

All publications patent applications mentioned are hereby incorporated by reference in their entirety as if each individual publication or patent application was specifically and individually indicated to be incorporated by reference. In case of conflict, the present application, including any definitions, will control.

1. A method for generating a sequencing sample comprising indexed sequencing templates, the method comprising subjecting a sample comprising indexed sequencing templates and extendable free or buried primers to a process that reduces the concentration of free or buried primers relative to the concentration of indexed sequencing templates to generate a sequencing sample that is less prone to index hopping when subjected to a next generation sequencing (NGS) assay.

2. The method of claim 1, wherein the indexed sequencing templates are indexed amplification products.

3. The method of claim 1, wherein the indexed sequencing templates comprise unique dual index (UDI) sequences.

4. The method of claim 1, wherein the indexed sequencing templates together comprise at least 100 unique barcode sequences.

5. The method of claim 1, wherein the method further comprises performing a next generation sequencing (NGS) assay on the sequencing sample.

6. The method of claim 1, wherein the process that reduces the relative concentration of extendable free or buried primers comprises performing high pressure liquid chromatography (HPLC).

7. The method of claim 6, wherein the HPLC is performed under denaturing conditions.

8. The method of claim 1, wherein the process that reduces the relative concentration of extendable free or buried primers comprises contacting the indexed sequencing template with terminal deoxy transferase (TdT) and dideoxynucleotide triphosphates (ddNTPs).

9. The method of claim 8, further comprising contacting the indexed sequencing template with a reagent that frees buried primers.

10. The method of claim 9, wherein the reagent that frees buried primers is a protein reagent.

11. The method of claim 10, wherein the protein that frees buried primers is single stranded binding protein (SSB), recA, or UvrB.

12. The method of claim 1, wherein the process that reduces the relative concentration of free or buried primers comprises contacting the indexed sequencing template with a killer oligonucleotide and a ligase, wherein the killer oligonucleotide comprises a region having a sequence complementary to that of a region of the primer, and wherein when the killer oligonucleotide is hybridized to the primer, the ligase is capable of ligating the killer oligonucleotide to the primer.

13. The method of claim 12, wherein the killer oligonucleotide comprises a 5′ phosphate.

14. The method of claim 12, wherein the killer oligonucleotide comprises a 3′ ddNTP.

15. The method of claim 12, wherein the ligase is TAQ ligase.

16. The method of claim 1, wherein the process that reduces the relative concentration of extendable free or buried primers comprises contacting the indexed sequencing template with a scavenger nucleic acid molecule, wherein the scavenger nucleic acid molecule comprises a region having a sequence complementary to that of a region of the primer.

17. The method of claim 16, wherein the scavenger nucleic acid molecule comprises a 3′ ddNTP.

18. The method of claim 1, wherein the process that reduces the relative concentration of extendable free or buried primers comprises (i) performing an amplification reaction on the indexed sequencing template using primers comprising a capture moiety to produce a capture moiety-tagged amplification product, and (ii) purifying the capture moiety-tagged amplification product.

19. The method of claim 18, wherein the capture moiety comprises biotin.

20. A sequencing sample generated according to the method of claim 1.
